(19) United States
(12) Patent Application Publication
(10) Pub. No.: US 2010/0248365 A1
(43) Pub. Date: Sep. 30, 2010

(54) GANGLIOSIDE BIOSYNTHESIS MODULATORS

(75) Inventors: Brett E. CRAWFORD, Poway, CA (US); Charles A. GLASS, San Diego, CA (US); Jillian R. BROWN, Poway, CA (US); Xiaomei BAI, San Diego, CA (US)

Correspondence Address: WILSON, SONSINI, GOODRICH & ROSATI, 650 PAGE MILL ROAD, PALO ALTO, CA, 94304-1050, US

(73) Assignee: ZACHARON PHARMACEUTICALS, INC., San Diego, CA (US)

(21) Appl. No.: 12/748,948

(22) Filed: Mar. 29, 2010

Related U.S. Application Data
(60) Provisional application No. 61/164,280, filed on Mar. 27, 2009.
(60) Provisional application No. 61/258,161, filed on Nov. 4, 2009.
(60) Provisional application No. 61/290,380, filed on Dec. 28, 2009.

(51) Int. Cl. C12N 5/07; C07H 15/06

(52) U.S. Cl. 435/375; 536/18.2

(57) ABSTRACT

Provided herein are ganglioside synthesis inhibitors, including modulators of ganglioside glycosylation.

### CROSS-REFERENCE

This application claims the benefit of U.S. Provisional Application No. 61/164,280, filed 27 Mar. 2009, U.S. Provisional Application No. 61/258,161, filed on 4 Nov. 2009, and U.S. Provisional Application No. 61/290,380, filed on 28 Dec. 2009, which applications are incorporated herein by reference.

### STATEMENT AS TO FEDERALLY SPONSORED RESEARCH

Certain inventions described herein were made with the support of the United States government under Contract 1 R43 CA119801 by the National Institutes of Health.

### BACKGROUND OF THE INVENTION

Glycolipids are lipid (e.g., ceramide) linked glycans that are found in mammals. In some instances, glycolipids comprise galactosyl, glucosyl and/or lactosyl residues attached to ceramide. In certain instances, a glycolipid is a ganglioside and comprises sialic acid residues.

### SUMMARY OF THE INVENTION

Described herein are ganglioside synthesis inhibitors, strategies for identifying and developing ganglioside synthesis inhibitors, methods for modifying the structures of gangliosides (including those on cells), methods for modulating the biosynthesis of gangliosides, methods for inhibiting ganglioside function, and methods for treating diseases associated with ganglioside signaling or ganglioside structure (including cancer and lysosomal storage diseases).

Provided in certain embodiments herein is a process for modifying the cellular population of a ganglioside, the process comprising contacting a cell having at least one ganglioside with an effective amount of a selective late-stage ganglioside biosynthesis inhibitor, the selective ganglioside biosynthesis inhibitor being active in a mammalian cell. In some embodiments, the selective late-stage ganglioside biosynthesis inhibitor utilized in any process described herein is a non-carbohydrate inhibitor. In certain embodiments, the selective ganglioside biosynthesis inhibitor utilized in any process herein has a molecular weight of less than 700 g/mol.

In some embodiments, any process described herein reduces the ratio of gangliosides containing mono (α 2,3) sialylation of the (β 1,4) galactose residue in the ceramide linked core compared to gangliosides containing no sialylation of the (β 1,4) galactose residue in the ceramide linked core, and/or reduces the ratio of gangliosides containing mono (α 2,3) sialylation of the (β 1,4) galactose residue in the ceramide linked core compared to gangliosides containing a di-sialylation of the (β 1,4) galactose residue in the ceramide linked core. In specific embodiments, the process reduces the cellular population of GM₁ gangliosides, GM₂ gangliosides, GM₃ gangliosides or a combination.

In certain embodiments, any process described herein reduces the ratio of gangliosides containing di-sialylation of the (β 1,4) galactose residue in the ceramide linked core compared to gangliosides containing no sialylation of the (β 1,4) galactose residue in the ceramide linked core, and/or reduces the ratio of gangliosides containing di-sialylation of the (β 1,4) galactose residue in the ceramide linked core compared to gangliosides containing mono (α 2,3) sialylation of the (β 1,4) galactose residue in the ceramide linked core. In specific embodiments, the process reduces the cellular population of GD_{1b}, GD₂ gangliosides, GD₃ gangliosides, or a combination.

In some embodiments, the selective ganglioside biosynthesis inhibitor utilized in any process described herein inhibits ST3Gal-V transferase, β1-4 GalNAc transferase, β1-3Gal-II transferase ST3Gal-I/II transferase, ST8Sial-I transferase, or a combination thereof. In a specific embodiment, the selective ganglioside biosynthesis inhibitor utilized in any process described herein directly inhibits the ST3Gal-V transferase, β1-4 GalNAc transferase, β1-3Gal-II transferase ST3Gal-I/II transferase, ST8Sial-I transferase, or a combination thereof. In another specific embodiment, the selective ganglioside biosynthesis inhibitor utilized in any process herein indirectly inhibits the ST3Gal-V transferase, β1-4 GalNAc transferase, β1-3Gal-II transferase ST3Gal-I/II transferase, ST8Sial-I transferase, or a combination thereof.

In certain embodiments, any process described herein reduces the ratio of gangliosides containing a terminal (β1,4) linked GalNAc linked to the (β1,4) galactose residue compared to gangliosides with a (β1,4) galactose lacking a GalNAc. In some embodiments, any process described herein reduces the ratio of gangliosides containing an unmodified (β1,3) linked galactose compared to gangliosides containing a terminal (β1,4) GalNAc.

In some embodiments, the cell contacted by any process described herein is a cancer cell or a cell having abnormal ganglioside accumulation. In certain embodiments, the cell is present in an individual diagnosed with or suspected of having cancer, inflammation or an inflammatory disease, pathogen entry, or lysosomal storage disease. In some embodiments, the cell is present in an individual diagnosed with or suspected of having melanoma, neuroblastoma, breast cancer or lung cancer. In certain embodiments, the cell is present in an individual diagnosed with or suspected of having a lysosomal storage disease, the lysosomal storage disease being Tay-Sachs, Sandhoff, AB variant, GM1 gangliosidosis, or Neimann-Pick.

In certain embodiments, disclosed herein is a composition comprising a population of human serum gangliosides, the population comprising less than 34 mol. %, less than 33 mol. %, less than 32 mol %, less than 31 mol. %, 30 mol. %, less than 25 mol. %, less than 20 mol. %, less than 15 mol. %, less than 10 mol. %, less than 5 mol. %, less than 2 mol. %, or less than 1 mol. % a 2,8-linked sialic acid containing gangliosides. Furthermore, as used herein, mol. % is the molar percentage of the selected ganglioside component compared to the total number of ganglioside components in the ganglioside(s) present and/or analyzed.

In some embodiments, disclosed herein is a composition comprising a population of human serum gangliosides, the population comprising greater than 3 mol. %, greater than 4 mol. %, greater than 5 mol. %, greater than 10 mol. %, greater than 15 mol. %, greater than 20 mol. %, greater than 30 mol. %, greater than 40 mol. %, greater than 50 mol. % 0 series gangliosides.

In certain embodiments, disclosed herein is a composition comprising a population of human serum gangliosides, the population comprising less than 15 mol. %, less than 10 mol. %, less than 5 mol. %, less than 2 mol. %, less than 1 mol. % (β1,3) linked galactose containing gangliosides.

In some embodiments, disclosed herein is a composition comprising a population of human serum gangliosides, the population comprising less than 23 mol. %, less than 22 mol. %, less than 21 mol. %, less than 20 mol. %, less than 15 mol. %, less than 10 mol. %, less than 5 mol. %, less than 2 mol. %, less than 1 mol. % (β1,4) linked GalNac gangliosides.

Provided in certain embodiments, herein is a process for modifying the structure of ganglioside on cells, comprising contacting a cell having at least one attached ganglioside moiety with a selective inhibitor of ganglioside biosynthesis, including a ganglioside glycosyltransferase.

Described herein is a process for modifying the structure of a GT_{1b} ganglioside, the process comprising contacting a cell having at least one GT_{1b} ganglioside with an effective amount of a selective inhibitor of GT_{1b} ganglioside biosynthesis.

In one embodiment is a process for modifying the structure of a GT_{1b} ganglioside, wherein the selective inhibitor of GT_{1b} ganglioside biosynthesis is an inhibitor of a sialyl transferase. In one embodiment the inhibitor of the sialyl transferase is an inhibitor of an α2,3-sialyltransferase, an α2,8-sialyl transferase or combination thereof. In another embodiment the selective inhibitor of GT_{1b} ganglioside biosynthesis is an inhibitor of an N-acetylgalactosaminyl transferase. In yet another embodiment the inhibitor of the N-acetylgalactosaminyl transferase is an inhibitor of a β1,4-N-acetylgalactosaminyl transferase. In a further embodiment the selective inhibitor of GT_{1b} ganglioside biosynthesis is an inhibitor of galactosyl transferase. In yet a further embodiment the inhibitor of galactosyl transferase is an inhibitor of β1,3-galactosyl transferase. In one embodiment the selective inhibitor of GT_{1b} ganglioside biosynthesis inhibits the addition of a NeuNAc residue to a nascent GT_{1b} ganglioside via an α2,3 linkage. In another embodiment the selective inhibitor of GT_{1b} ganglioside biosynthesis inhibits the addition of a NeuNAc residue to a nascent GT_{1b} ganglioside via an α2,8 linkage. In yet another embodiment the selective inhibitor of GT_{1b} ganglioside biosynthesis inhibits the addition of an N-acetylgalactosamine residue to a nascent GT_{1b} ganglioside via a β1,4 linkage. In a further embodiment the selective inhibitor of GT_{1b} ganglioside biosynthesis inhibits the addition of a galactose residue to a nascent GT_{1b} ganglioside via a β1,3 linkage. In yet a further embodiment the cell being contacted is a cell in need thereof, a cell present in an individual suffering from a disease or condition mediated by abnormal GT_{1b} biosynthesis and/or the cell itself is a cell with abnormal Gt_{1b} biosynthesis.

In one aspect is a process of modulating the biosynthesis of a GT_{1b} ganglioside in a subject comprising administering to the subject a therapeutically effective amount of an agent that reduces or inhibits the activity of an upstream regulator of a GT_{1b} upstream ganglioside.

In one embodiment the agent is a selective inhibitor of lactosylceramide synthase. In another embodiment the GT_{1b} upstream ganglioside is selected from G_{M3}, G_{D3}, G_{D2}, and G_{D1b}.

Also described herein is a process for modifying the structure of a ganglioside, the process comprising contacting a cell having at least one ganglioside with an effective amount of a selective inhibitor of ganglioside biosynthesis.

In one embodiment the selective inhibitor of ganglioside biosynthesis is an inhibitor of a sialyl transferase, an N-acetylgalactosaminyl transferase, a galactosyl transferase, or a combination thereof. In another embodiment the inhibitor of the sialyl transferase is an inhibitor of an α2,3-sialyl transferase, an α2,6-sialyl transferase, an α2,8-sialyl transferase, or combination thereof. In yet another embodiment the inhibitor of the N-acetylgalactosaminyl transferase is an inhibitor of a β1,4-N-acetylgalactosaminyl transferase. In a further embodiment the inhibitor of the galactosyl transferase is an inhibitor of β1,3-galactosyl transferase. In yet a further embodiment the selective inhibitor of ganglioside biosynthesis inhibits the addition of a NeuNAc residue to a nascent ganglioside via an α2,3 linkage. In one embodiment the selective inhibitor of ganglioside biosynthesis inhibits the addition of a NeuNAc residue to a nascent ganglioside via an α2,6 linkage. In another embodiment the selective inhibitor of ganglioside biosynthesis inhibits the addition of a NeuNAc residue to a nascent ganglioside via an α2,8 linkage. In yet another embodiment the selective inhibitor of ganglioside biosynthesis inhibits the addition of an N-acetylgalactosamine residue to a nascent ganglioside via a β1,4 linkage. In a further embodiment the selective inhibitor of ganglioside biosynthesis inhibits the addition of a galactose residue to a nascent ganglioside via a β1,3 linkage.

In yet a further embodiment the selective inhibitor of ganglioside biosynthesis inhibits the addition of a NeuNAc residue to a ganglioside having the structure:

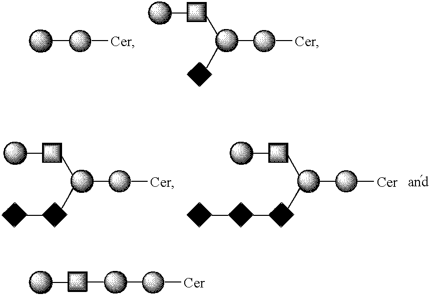

via an α2,3 linkage.

In one embodiment the selective inhibitor of ganglioside biosynthesis inhibits the addition of a NeuNAc residue to a ganglioside having the structure:

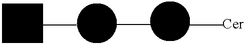

via an α2,3 linkage.

In another embodiment the selective inhibitor of ganglioside biosynthesis inhibits the addition of a NeuNAc residue to a ganglioside having the structure:

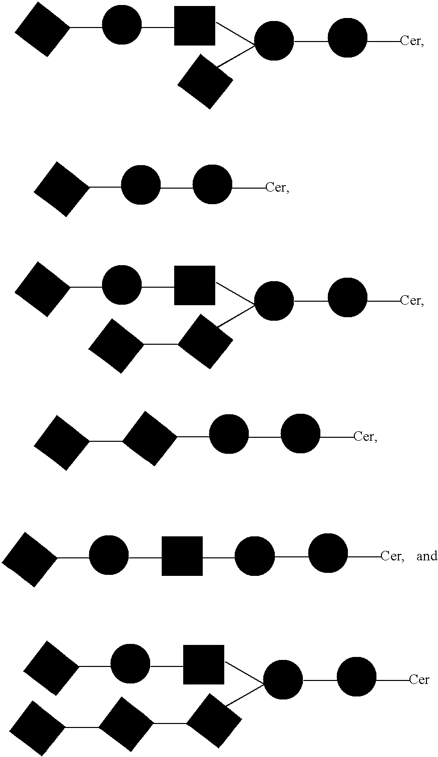

via an α2,8 linkage.

In yet another embodiment the selective inhibitor of ganglioside biosynthesis inhibits the addition of an N-acetylgalactosamine residue to a ganglioside having the structure:

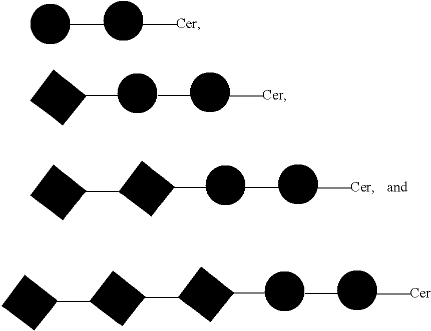

via an β1,4 linkage.

In a further embodiment the selective inhibitor of ganglioside biosynthesis inhibits the addition of a galactose residue to a ganglioside having the structure:

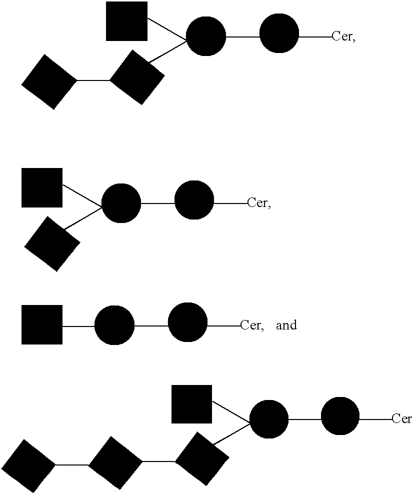

via an β1,3 linkage.

In yet a further embodiment the cell being contacted is a cell in need thereof, a cell present in an individual suffering from a disease or condition mediated by abnormal ganglioside biosynthesis and/or the cell itself is a cell with abnormal ganglioside biosynthesis, a cell present in an individual with normal ganglioside biosynthesis and/or the cell itself is a cell with normal ganglioside biosynthesis. In some embodiments, the cell being contacted is a cell present in an individual with normal ganglioside biosynthesis (e.g., an individual with a predisposition for or suspected of having a disease or condition mediated by ganglioside biosynthesis) and/or the cell itself is a cell with normal ganglioside biosynthesis.

In one aspect is a process of inhibiting ganglioside function in a cell comprising contacting the cell with an effective amount of a selective modulator of a sialyl transferase, an N-acetylgalactosaminyl transferase, a galactosyl transferase or a combination thereof.

In one embodiment the ganglioside function inhibited is an ability to modulate the activity of a receptor tyrosine kinase. In another embodiment the receptor tyrosine kinase is an EGF receptor. In yet another embodiment the ganglioside function inhibited is an ability to modulate the activity of a nerve growth factor receptor. In a further embodiment the cell being contacted is a cell in need thereof, a cell present in an individual suffering from a disease or condition mediated by abnormal ganglioside biosynthesis and/or the cell itself is a cell with abnormal ganglioside biosynthesis, a cell present in an individual with normal ganglioside biosynthesis and/or the cell itself is a cell with normal ganglioside biosynthesis. In some embodiments, the cell being contacted is a cell present in an individual with normal ganglioside biosynthesis (e.g., an individual with a predisposition for or suspected of having a disease or condition mediated by ganglioside biosynthesis) and/or the cell itself is a cell with normal ganglioside biosynthesis.

Also presented herein is a process of inhibiting ganglioside function in a cell comprising contacting the cell with an effective amount of a selective modulator of ganglioside biosynthesis.

In one embodiment the selective modulator of ganglioside biosynthesis inhibits sialylation of a ganglioside. In another embodiment the selective modulator of ganglioside biosynthesis inhibits galactosylation of a ganglioside. In yet another embodiment the selective modulator of ganglioside biosynthesis inhibits N-acetylgalactosaminylation of a ganglioside. In a further embodiment the selective modulator of ganglioside biosynthesis is an inhibitor of a sialyl transferase. In one embodiment the selective modulator of ganglioside biosynthesis is a promoter of a sialyl transferase. In yet a further embodiment the selective modulator of ganglioside biosynthesis is an inhibitor of a galactosyl transferase. In one embodiment the selective modulator of ganglioside biosynthesis is a promoter of a galactosyl transferase. In another embodiment the selective modulator of ganglioside biosynthesis is an inhibitor of an N-acetylgalactosaminyl transferase. In yet another embodiment the selective modulator of ganglioside biosynthesis is a promoter of an N-acetylgalactosaminyl transferase. In a further embodiment the cell is present in a human diagnosed with cancer.

Described herein is a process of normalizing and/or modulating the biosynthesis of a ganglioside in a subject suffering from abnormal ganglioside biosynthesis, the process comprising administering to the subject a therapeutically effective amount of an agent that modulates the activity of an upstream regulator of the ganglioside.

In one embodiment the agent is a selective modulator of GlcCer synthase, lactosylceramide synthase, or a combination thereof. In another embodiment the ganglioside is selected from G_{A2}, G_{A1}, G_{M1b}, G_{D1α}, G_{T1aα}, G_{Q1bα}, G_{D1c}, G_{M2}, G_{M3}, G_{M2α}, G_{M1}, G_{D1a}, G_{T1a}, G_{D3}, G_{D2}, G_{D1b}, G_{T1b}, G_{Q1b}, G_{T3}, G_{T2}, G_{T1c}, G_{Q1c}, and G_{P1c}.

Also provided herein is a method of treating cancer in a subject comprising administering to the subject a therapeutically effective amount of a selective modulator of ganglioside biosynthesis.

In one embodiment the selective modulator of ganglioside biosynthesis is a modulator of a sialyl transferase, an N-acetylgalactosaminyl transferase, a galactosyl transferase or a combination thereof. In yet another embodiment the selective modulator of ganglioside biosynthesis is an inhibitor of an α2,3-sialyl transferase, an α2,6-sialyl transferase, an α2,8-sialyl transferase, or combination thereof. In a further embodiment the selective modulator of ganglioside biosynthesis is an inhibitor of a β1,4-N-acetylgalactosaminyl transferase. In yet a further embodiment the selective modulator of ganglioside biosynthesis is an inhibitor of β1,3-galactosyl transferase. In another embodiment the cancer is neuroblastoma or melanoma.

In one aspect method of treating a lysosomal storage disease comprising administering a therapeutically effective amount of a selective inhibitor of ganglioside biosynthesis.

In one embodiment the selective inhibitor of ganglioside biosynthesis is a selective modulator of a sialyl transferase, an N-acetylgalactosaminyl transferase, a galactosyl transferase, or combination thereof. In one embodiment the lysosomal storage disease is Salidosis, Tay Sachs, Sandhoff, or GM₁ gangliosidosis. In yet another embodiment the lysosomal storage disease is Fabry disease.

The present disclosure provides a process for modulating ganglioside degradation in a cell comprising contacting the cell with an effective amount of a selective modulator of a glucocerebrosidase, a β-galactosidase, or combination thereof.

In one embodiment the selective modulator of the glucocerebrosidase is a promoter of glucocerebrosidase. In another embodiment the selective modulator of the β-galactosidase is a selective modulator of β-galactoceramidase. In a further embodiment the selective modulator of β-galactoceramidase is a promoter of β-galactoceramidase. In yet a further embodiment the cell being contacted is a cell in need thereof, a cell present in an individual suffering from a disease or condition mediated by abnormal ganglioside biosynthesis and/or the cell itself is a cell with abnormal ganglioside biosynthesis.

Also provided herein is a process for identifying a compound that modulates ganglioside biosynthesis comprising:
a. contacting a mammalian cell with the compound;
b. contacting the mammalian cell and compound combination with a first labeled probe wherein the first labeled probe binds one or more gangliosides;
c. incubating the mammalian cell, compound, and the first labeled probe;
d. collecting the first labeled probe that is bound to one or more gangliosides; and
e. detecting or measuring the amount of first labeled probe bound to one or more gangliosides.

Further provided herein is a process for identifying a compound that selectively modulates ganglioside biosynthesis comprising:
a. contacting a mammalian cell with the compound;
b. contacting the mammalian cell and compound combination with a first labeled probe and a second labeled probe, wherein the first labeled probe binds one or more gangliosides and the second labeled probe binds at least one glycan other than a ganglioside or specific type of targeted ganglioside (i.e., other than the one or more ganglioside);
c. incubating the mammalian cell, compound, the first labeled probe, and the second labeled probe;
d. collecting the first labeled probe that is bound to one or more gangliosides;
e. collecting the second labeled probe that is bound to at least one glycan other than a ganglioside or specific type of targeted ganglioside (i.e., other than the one or more ganglioside);
f. detecting or measuring the amount of first labeled probe bound to one or more gangliosides; and
g. detecting or measuring the amount of the second labeled probe bound to at least one glycan other than a ganglioside or specific type of targeted ganglioside (i.e., other than the one or more ganglioside).

In some embodiments, the mammalian cell is a human melanoma cancer cell. In some embodiments, the labeled probe comprises a biotinyl moiety and the process further comprises tagging the labeled probe with streptavidin-Cy5-PE. In some embodiments, the labeled probe comprises a fluorescent label. In some embodiments, the first labeled probe is a labeled protein. In some embodiments, the labeled protein is a ganglioside-specific lectin. In some embodiments, the second labeled probe is a labeled lectin. In some embodiments, the labeled lectin is a lectin specific or a glycan other than a ganglioside or specific type of targeted ganglioside (i.e., other than the one or more ganglioside).

Also provided herein is a glycolipid comprising a lipid covalently linked to at least one ganglioside, wherein the at least one ganglioside comprises a plurality of O series, A series, B series or C series gangliosides, and wherein less than 20% of the plurality of gangliosides are GT_{1b} gangliosides.

Further provided herein is a glycolipid comprising a lipid covalently linked to at least one ganglioside, wherein the at least one ganglioside comprises a plurality of O series, A series, B series or C series gangliosides, and wherein less than 20% of the plurality of gangliosides are GM₁ₐ gangliosides.

Also provided herein is a glycolipid comprising a lipid covalently linked to at least one ganglioside, wherein the at least one ganglioside comprises a plurality of O series, A series, B series or C series gangliosides, and wherein less than 34%, less than 33%, less than 32%, less than 31%, 30%, less than 20%, less than 10%, less than 5%, less than 2%, or less than 1% of the plurality of gangliosides are α-2,8-linked sialic acid gangliosides.

Further provided herein is a glycolipid comprising a lipid covalently linked to at least one ganglioside, wherein the at least one ganglioside comprises a plurality of O series, A series, B series or C series gangliosides, and wherein less than 23%, less than 22%, less than 21%, less than 20%, less than 15%, less than 10%, less than 5%, less than 2%, or less than 1% of the plurality of gangliosides are β-1,4-linked GalNAc gangliosides.

Further provided herein is a glycolipid comprising a lipid covalently linked to at least one ganglioside, wherein the at least one ganglioside comprises a plurality of O series, A series, B series or C series gangliosides, and wherein less than 15%, less than 10%, less than 5%, less than 2%, less than 1% of the plurality of gangliosides are β-1,3-linked galactose containing gangliosides.

Further provided herein is a glycolipid comprising a lipid covalently linked to at least one ganglioside, wherein the at least one ganglioside comprises a plurality of O series, A series, B series or C series gangliosides, and wherein greater than 3%, greater than 4%, greater than 5%, greater than 10%, greater than 15%, greater than 20%, greater than 30%, greater than 40%, greater than 50% of the plurality of gangliosides are O series gangliosides.

In certain embodiments, provided herein is a process for modifying the cellular population of gangliosides, the process comprising contacting a cell having at least one ganglioside with an effective amount of a selective inhibitor of β-1,4-N-acetylgalactosaminyl transferase. In some embodiments, the selective inhibitor of β-1,4-N-acetylgalactosaminyl transferase is a selective cellularly active non-carbohydrate inhibitor of β-1,4-N-acetylgalactosaminyl transferase. In certain embodiments, the process modifies the cellular population of gangliosides to provide an increased ganglioside ratio in the cell of GD₃ or GM₃, compared to GA₂, GA₁, GM₁, GT_{1b}, or GQ_{1b} (e.g., by a factor of 1.1, of 1.2, of 1.3, of 1.5, of 2, of 3, of 5, of 10, of 20, of 30, of 50, or greater; for example, an increase by a factor of 1.1 would indicate a change in a ratio of 1:1 to a ratio of 1.1:1).

In some embodiments, provided herein is a process for modifying the cellular population of gangliosides, the process comprising contacting a cell having at least one ganglioside with an effective amount of a selective inhibitor of β-1,3-GalT2. In certain embodiments, the selective inhibitor of β-1,3-GalT2 is a selective cellularly active non-carbohydrate inhibitor of β-1,3-GalT2. In some embodiments, process modifies the cellular population of gangliosides to provide an increased ganglioside ratio in the cell of GD₂, GA₂, GM₃ or GM₂, compared to, GA₁, GM₁, GT_{1b}, or GQ_{1b} (e.g., by a factor of 1.1, of 1.2, of 1.3, of 1.5, of 2, of 3, of 5, of 10, of 20, of 30, of 50, or greater).

In certain embodiments, provided herein is a process for modifying the cellular population of gangliosides, the process comprising contacting a cell having at least one ganglioside with an effective amount of a selective inhibitor of ST3GalII. In some embodiments, the selective inhibitor of ST3GalII is a selective cellularly active non-carbohydrate inhibitor of ST3GalII. In certain embodiments, the process modifies the cellular population of gangliosides to provide an increased ganglioside ratio in the cell of GD_{1b}, GA₁, GM₁ or GM₃, compared to, GM_{1b}, GD₁ₐ, GT_{1b}, or GQ_{1b} (e.g., by a factor of 1.1, of 1.2, of 1.3, of 1.5, of 2, of 3, of 5, of 10, of 20, of 30, of 50, or greater).

In some embodiments, provided herein is a process for modifying the cellular population of gangliosides, the process comprising contacting a cell having at least one ganglioside with an effective amount of a selective inhibitor of CMP-NeuAc:lactosylceramide α2,3-sialyltransferase (GM₃ synthase). In certain embodiments, the selective inhibitor of CMP-NeuAc:lactosylceramide α2,3-sialyltransferase is a selective cellularly active non-carbohydrate inhibitor of CMP-NeuAc:lactosylceramide α2,3-sialyltransferase. In some embodiments, the process modifies the cellular population of gangliosides to provide an increased ganglioside ratio in the cell of GA₁, GA₂, or GM_{1b}, compared to, GM₃, GD₃, GM₁, or GT_{1b} (e.g., by a factor of 1.1, of 1.2, of 1.3, of 1.5, of 2, of 3, of 5, of 10, of 20, of 30, of 50, or greater). In certain embodiments, the process modifies cellular population of GM₃, GM₂, GM₁, GD₃, GD₁ₐ, and/or GD_{1b} that is reduced by greater than 10%, greater than 15%, greater than 25%, greater than 40%, or greater than 60% compared to the cellular population prior to contact with the selective inhibitor of CMP-NeuAc:lactosylceramide α2,3-sialyltransferase.

In certain embodiments, provided herein is a process for modifying the cellular population of gangliosides, the process comprising contacting a cell having at least one ganglioside with an effective amount of a selective inhibitor of GD3 synthase (ST8Sial-T1). In some embodiments, the selective inhibitor of ST8Sial-T1 is a selective cellularly active non-carbohydrate inhibitor of ST8Sial-T1. In certain embodiments, the process modifies the cellular population of gangliosides to provide an increased ganglioside ratio in the cell of GM₃, GM₂, GM₁ or GD₁ₐ, compared to, GD₃, GD₂, GT_{1b}, or GQ_{1b} (e.g., by a factor of 1.1, of 1.2, of 1.3, of 1.5, of 2, of 3, of 5, of 10, of 20, of 30, of 50, or greater).

In some embodiments, provided herein is a process for modifying the cellular population of gangliosides, the process comprising contacting a cell having at least one ganglioside with an effective amount of a selective inhibitor of lactosylceramide synthase (β-1,4-GalT1). In certain embodiments, the selective inhibitor of lactosylceramide synthase is a selective cellularly active non-carbohydrate inhibitor of lactosylceramide synthase. In some embodiments, the process modifies cellular population of gangliosides to provide a increase in ratio of GlcCer relative to LacCer, increase in ratio of GlcCer relative to one or more LacCer downstream ganglioside, increase in ratio of GlcCer relative to Muco series gangliosides, increase in ratio of GlcCer relative to globo series gangliosides, increase in ratio of GlcCer relative to isoglobo series gangliosides, increase in ratio of GlcCer relative to lacto series gangliosides, increase in ratio of GlcCer relative to neo-lacto series gangliosides, or a combination thereof (e.g., by a factor of 1.1, of 1.2, of 1.3, of 1.5, of 2, of 3, of 5, of 10, of 20, of 30, of 50, or greater).

In some embodiments, provided herein is a ganglioside or ganglioside composition prepared according to a process described herein.

Other objects and features of the methods, compositions and uses described herein will become apparent from the following detailed description. It should be understood, however, that the detailed description and the specific examples, while indicating specific embodiments, are given by way of illustration only.

### BRIEF DESCRIPTION OF THE DRAWINGS

The features disclosed herein are set forth with particularity in the appended claims. A better understanding of the features and advantages of the embodiments disclosed herein will be obtained by reference to the following detailed description that sets forth illustrative embodiments, in which the principles of the embodiments are utilized, and the accompanying drawings.

FIG. 1 illustrates two major classes of GSLs.

FIG. 2 illustrates flow cytometry showing GM1 binding with cholera toxin B-subunit (CTB-Bio).

FIG. 3 illustrates a representative TLC for quantification of gangliosides.

FIG. 4 illustrates the type and quantity of gangliosides produced by cells.

FIG. 5 illustrates the cellular inhibition of ganglioside biosynthesis by compound 1.

FIG. 6 illustrates the cellular inhibition of ganglioside biosynthesis by various compounds.

FIG. 7 illustrates the cellular inhibition of ganglioside biosynthesis by various compounds.

FIG. 8 illustrates the cellular inhibition of specific ganglioside biosynthesis by PDMP.

FIG. 9 illustrates the cellular inhibition of specific ganglioside biosynthesis by PDMP.

FIG. 10 illustrates the cellular inhibition of specific ganglioside biosynthesis by compound 17.

FIG. 11 illustrates the cellular inhibition of specific ganglioside biosynthesis by compound 17.

FIG. 12 illustrates the cellular inhibition of specific ganglioside biosynthesis by compound 2.

FIG. 13 illustrates the cellular inhibition of specific ganglioside biosynthesis by compound 2.

FIG. 14 illustrates the cellular inhibition of specific ganglioside biosynthesis by compound 15.

FIG. 15 illustrates the cellular inhibition of specific ganglioside biosynthesis by compound 15.

FIG. 16 illustrates the preferential reduction effect of compound 3 on gangliosides GM₃ and GD₃.

FIG. 17 illustrates the preferential reduction effect of compound 8 on gangliosides GM₃ and GD₃.

FIG. 18 illustrates the preferential reduction effect of compound 5 on 2 series gangliosides GM₂ and GD₂ relative to the 3 series gangliosides GM₃ and GD₃.

FIG. 19 illustrates the reduction in B series gangliosides relative to A series gangliosides by compound 4.

FIG. 20 illustrates the dose dependent reduction effect of compound 17 on ganglioside GM₃.

FIG. 21 illustrates the dose dependent reduction effect of compound 18 on ganglioside GM₂.

FIG. 22 illustrates the dose dependent reduction effect of compound 19 on ganglioside GM₁.

FIG. 23 illustrates the dose dependent reduction effect of compound 4 on ganglioside GD₃.

FIG. 24 illustrates the dose dependent reduction effect of compound 5 on ganglioside GD₂.

FIG. 25 illustrates the dose dependent reduction effect of compound 2 on ganglioside GD_{1b}.

FIG. 26 illustrates the reduction of GM₂ storage in primary human fibroblasts from Sandhoff patients by the non-selective glycolipid inhibitor PDMP.

FIG. 27 illustrates the reduction of GM₂ storage in primary human fibroblasts from Sandhoff patients by the non-selective glycolipid inhibitor DGNJ.

FIG. 28 illustrates the reduction of GM₂ storage in primary human fibroblasts from Sandhoff patients by compound 20.

FIG. 29 illustrates the reduction of GM₂ storage in primary human fibroblasts from Sandhoff patients by compound 3.

FIG. 30 illustrates the reduction of GM₂ storage in primary human fibroblasts from Sandhoff patients by compound 5.

FIG. 31 illustrates the reduction of GM₂ storage in primary human fibroblasts from Sandhoff patients by compound 7.

FIG. 32 illustrates the reduction of GM₂ storage in primary human fibroblasts from Sandhoff patients by compound 19.

FIG. 33 illustrates the reduction of GM₂ storage in primary human fibroblasts from Sandhoff patients by compound 17.

FIG. 34 illustrates the reduction of GM₂ storage in primary human fibroblasts from Sandhoff patients by compound 8.

FIG. 35 illustrates the reduction of GM₂ storage in primary human fibroblasts from Tay-Sachs patients by compound 21.

FIGS. 36A-36I illustrate selective modulators (e.g., inhibitors or promoters) of ganglioside biosynthesis.

### DETAILED DESCRIPTION OF THE INVENTION

### Ganglioside Synthesis Inhibitors

Provided in certain embodiments herein are glycolipid synthesis inhibitors. In some instances, glycolipids comprise ceramide-linked glycans. In certain instances, glycolipids are gangliosides and comprise ceramide-linked glycans that are linked to one or more sialic acid residues. In certain instances, glycolipid synthesis inhibitors are ganglioside synthesis inhibitors. In general, ganglioside synthesis inhibitors modulate or alter the nature (e.g., character, structure, or concentration) of gangliosides (e.g., the endogenous ganglioside, or in/on a cell, tissue, organ or individual). Within the class of glycolipids described as gangliosides, there is broad variability with respect to the ceramide moiety (e.g., variation in number of unsaturated bonds and/or hydroxylation and/or length (e.g., C₁₄-C₂₄) of fatty acid chain), position and/or linkage (e.g., linear, branched) of saccharide units of the glycan, location and degree of sialylation of the glycan, and other modifications.

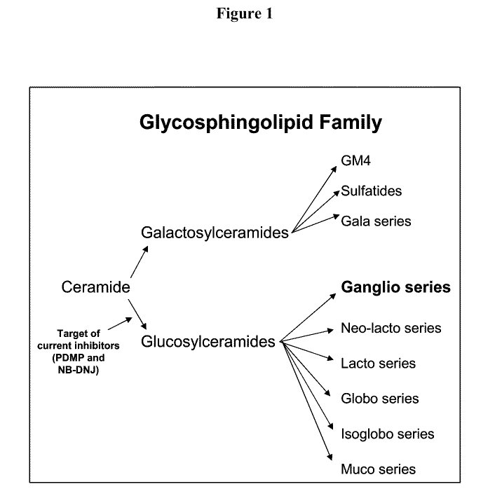

Glycosphingolipids (GSLs) are lipid (ceramide) linked glycans that are present on the extracellular surface of eukaryotic cells. There are two major classes of GSLs, those based on galactosylceramide and those built upon glucosylceramide (see FIG. 1). In mammalian systems, the glucosylceramide based GSLs are the most common; the glucose molecule is typically substituted with beta-linked galactose on the C-4 hydroxyl of glucose to give the lactosylceramide (Galbeta1-4GlcbetaCer). Further extensions of the glycan generate a series of neutral “core” structures that form the basis for the nomenclature glycosphingolipids. The ganglio series of glycosphingolipids are based on the neutral core structure: Galbeta1-3GalNAcbeta1-4GlcbetaCer. Traditionally, all sialylated glycosphingolipids are known as gangliosides, regardless of whether they are based on the ganglio-series of neutral core structure. Gangliosides are found throughout the body; however, they are most abundant in the brain. In various instances, gangliosides include o, a, b, and c series gangliosides (including, by way of non-limiting example, sialated glycolipids). In some instances, gangliosides include GA2 and GA1 gangliosides (which are not sialated). In other instances, gangliosides as referred to herein globo series gangliosides (e.g., that are sialated). In specific embodiments, gangliosides described herein include, e.g., G_{A2}, G_{A1}, G_{M1b}, G_{D1α}, G_{T1aα}, G_{Q1bα}, G_{D1c}, G_{M2}, G_{M3}, G_{M2α}, G_{M1}, G_{D1a}, G_{T1a}, G_{D3}, G_{D2}, G_{D1b}, G_{T1b}, G_{Q1b}, G_{T3}, G_{T2}, G_{T1c}, G_{Q1c}, and G_{P1c} gangliosides. Therefore, provided herein are modulators (e.g., selective modulators, such as selective inhibitors or selective promoters) of one or more of G_{A2}, G_{A1}, G_{M1b}, G_{D1α}, G_{T1aα}, G_{Q1bα}, G_{D1c}, G_{M2}, G_{M3}, G_{M2α}, G_{M1}, G_{D1a}, G_{T1a}, G_{D3}, G_{D2}, G_{D1b}, G_{T1b}, G_{Q1b}, G_{T3}, G_{T2}, G_{T1c}, G_{Q1c}, and G_{P1c} ganglioside biosynthesis.

Ganglioside biosynthesis may occur in a stepwise fashion, with individual sugars added first to ceramides and subsequent sugars transferred by glycosyltransferases from nucleotide sugar donors. Ceramide may be synthesized on the cytoplasmic face of the endoplasmic reticulum (ER); it subsequently equilibrates to the luminal face and traffics to the Golgi compartment. GlcCer may be synthesized on the cytoplasmic face of the endoplasmic reticulum and early Golgi apparatus; it then flips into the Golgi lumen, where it is elongated by a series of glycosyltransferases. Competing biosynthetic pathways may lead to ganglioside structural diversity. In the brain, gangliosides may be synthesized by all cells with concentrations of the different forms varying according to cell type.

In certain instances, ceramide is connected to a glycan via and/or comprising a linkage disaccharide, which generally has the structure -Galβ4Glcβ1-Ceramide, (Formula I and Ia).

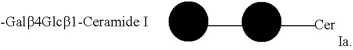

In various instances, the linkage disaccharide is further modified to one or more of the following cores: Galβ3GlcNAcβ3Galβ4Glcβ1Ceramide, Galβ4GlcNAcβ3Galβ4Glcβ1Ceramide, GalNAcβ3Galα4Galβ4Glcβ1Ceramide, GalNAcβ3Galα3Galβ4Glcβ1Ceramide, Galβ3GalNAcβ4Galβ4Glcβ1Ceramide, Galβ3Galβ3Galβ4Glcβ1Ceramide.

In some instances, ceramide is linked to galactosyl residues, e.g. Galα4Galβ1Ceramide, NeuAcα23Galβ1Ceramide and/or 3-O-Sulfo-Galβ1Ceramide. In various instances, one or more sialic acid residues are linked to a ceramide-linked glycan. In some instances, a branched GalNAc is attached to a -Galβ4Glcβ1-Ceramide linkage disaccharide of a glycan. In some instances, one or more sialic acid residues are attached to a branched GalNAc. In various instances, one or more sialic acid residues on a ceramide-linked glycan is sulfated e.g., at 3-OH and the like.

In some instances, a -Galβ4Glcβ1-Ceramide (lactosylceramide, LacCer) is linked with a sialic acid residue (NeuNAc) e.g., α2-3 to a galactosyl residue. In some instances, a second NeuNAc residue is attached to a first sialic acid residue e.g., α2-8 to a NeuAc residue. In some instances, a branched GalNAc is attached to a -Galβ4Glcβ1-Ceramide linkage disaccharide of a glycan. In some instances, one or more sialic acid residues are attached to a branched GalNAc. In some instances, a ganglioside is a GT_{1b} ganglioside of Formula II or IIa:

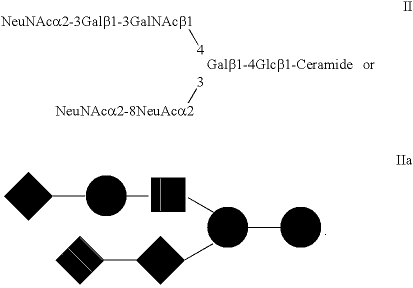

In some embodiments, glycolipid synthesis inhibitors described herein modulate glycolipid biosynthesis, e.g., initiation of the synthesis of ceramide (e.g., by 1-O-Acylceramide synthase), synthesis of a LacCer moiety, attachment of the linkage disaccharide to one or more of a glucosyl and/or galactosyl and/or sialic acid residues, glycan sulfation (N or O sulfation), glycan phosphorylation, and/or glycan acetylation (N or O acetylation). In some instances, modulation of glycolipid synthesis includes modulation of ganglioside synthesis. As utilized herein, modulation of ganglioside biosynthesis includes the modulation of ganglioside polymerization (e.g. with glucosyl, galactosyl and/or sialic acid residues), ganglioside sulfation (N or O sulfation), ganglioside phosphorylation, ganglioside acetylation (N or O acetylation), and/or ganglioside degradation. In some instances, modulation of ganglioside biosyntheses includes the promotion of one or more of and/or the inhibition of one or more of ganglioside polymerization, ganglioside sulfation, ganglioside phosphorylation, ganglioside acetylation and/or ganglioside degradation.

The modulation of ganglioside biosynthesis includes the modulation of the production of the disaccharide linkage region (e.g., -Galβ4Glcβ1-Ceramide) that connects a glycan to a ceramide. In certain embodiments, the modulation of the production of the linkage region includes the inhibition of the production of or synthesis of the linkage region. In certain instances, a ganglioside synthesis inhibitor described herein directly promotes production or cleavage, while in other instances, a ganglioside synthesis inhibitor impacts (including modifying the character of) an endogenous chemical (e.g., by activating or deactivating an enzyme) that inhibits production or promotes cleavage of the linkage region. In some embodiments, an inhibitor of ganglioside that modulates the production of the linkage region inhibits one or more ceramide synthases. In some embodiments, the synthase is a galactosylceramide synthase, a glucosylceramide synthase or a combination thereof. In some embodiments, the synthase is a lactosylceramide synthase (LacCer synthase).

The modulation of ganglioside biosynthesis includes the modulation of further modification of a disaccharide linkage region (e.g., -Galβ4Glcβ1-Ceramide) that connects a glycan to a ceramide. In some embodiments, an inhibitor of ganglioside synthesis modulates synthesis of B series gangliosides (e.g., a G_{T1b} ganglioside or the like). In some embodiments, an inhibitor of ganglioside synthesis modulates synthesis of O, A or C series gangliosides (e.g., G_{M1b} gangliosides, G_{D1a} gangliosides, G_{Q1c} gangliosides or the like). In certain embodiments, modulation of the modification of the linkage region includes the inhibition of glycosyl transferases or sialyl transferases. In certain instances, a ganglioside synthesis inhibitor described herein reduces or inhibits the activity of a glycosyl transferases or a sialyl transferase. In some instances, a ganglioside synthesis inhibitor impacts (including modifying the character of) an endogenous chemical (e.g., by activating or deactivating an enzyme) that inhibits or reduces the activity of a glycosyl or sialyl transferase. In some embodiments, an inhibitor of ganglioside synthesis modulates a glycosyl transferase or a sialyl transferase inhibits one or more of β-galactoside α-2,3-sialyltransferase (ST3), α-N-acetyl-neuraminide α-2,8-sialyltransferase 1 (ST8), (α-N-acetyl-neuraminyl-2,3-beta-galactosyl-1,3)-N-acetylgalactosaminide α-2,6-sialyltransferase 3 (ST6), (α-N-acetyl-neuraminyl-2,3-beta-galactosyl-1,3)-N-acetylgalactosaminide α-2,6-sialyltransferase 4 (ST6), (α-N-acetyl-neuraminyl-2,3-β-galactosyl-1,3)-N-acetylgalactosaminide α-2,6-sialyltransferase 5 (ST6), (α-N-acetyl-neuraminyl-2,3-β-galactosyl-1,3)-N-acetylgalactosaminide α-2,6-sialyltransferase 6 (ST6), β-1,4-N-acetyl-galactosaminyl transferase 1, UDP-Gal:βGlcNAc β1,3-galactosyltransferase, polypeptide 4, or a combination thereof. In certain instances, a ganglioside synthesis inhibitor impacts an endogenous chemical (e.g., by activating or deactivating an enzyme) that inhibits synthesis or promotes the activity of a ganglioside galactosyl transferase or a sialyl transferase.

In certain embodiments, the modulation of ganglioside biosynthesis includes modulation of degradation of gangliosides. In some embodiments, the modulation of degradation of gangliosides promotes and/or inhibits recycling of saccharide units used for glycan biosynthesis. In some embodiments, modulation of degradation of gangliosides includes modulation of endoglycosidases and/or exoglycosidases. In some embodiments, modulation of endoglycosidases and/or exoglycosidases includes the promotion and/or inhibition of a glucocerebrosidase, e.g., β-glucoceramidase, β-galactoceramidase, sialidase (e.g. neuraminidase), β-galactosidase, α-galactosidase, sulfatases (e.g. arylsulfatase A), and/or sphingomyelinase. In some embodiments, modulation of ganglioside degradation includes the promotion and/or inhibition of activator proteins (e.g., saposin A, saposin B) that mediate ganglioside degradation.

### Selectivity

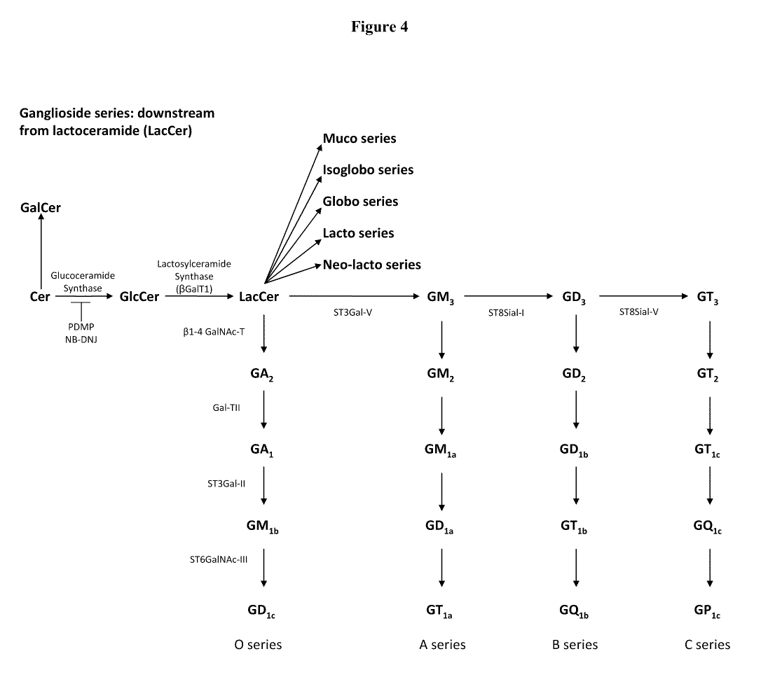

Early stage inhibitors have general, and in some cases, undesirable effects that a ganglioside inhibitor demonstrating greater specificity overcomes, in certain instances. Early stage inhibition of may, in certain instances, introduce non-specificity because all classes of glucoceramides are affected (see FIG. 4). As illustrated in FIG. 4, the glucosylceramide synthase may be essential for the synthesis of all glucosylceramide based GSLs, including the muco, isoglobo, globo, lacto, and neo-lacto families. Thus, in certain embodiments, provided herein are late stage ganglioside inhibitors (e.g., late stage ganglioside biosynthesis inhibitors). In some embodiments, the late stage ganglioside biosynthesis inhibitors inhibit one or more process in the late stage biosynthetic pathway, as described herein, but do not affect the biosynthesis of or gangliosides in biosynthetic pathway prior to the late stage biosynthetic pathway. In various embodiments, an agent that does not affect the biosynthesis of or gangliosides in biosynthetic pathway prior to the late stage biosynthetic pathway affects the non-late stage biosynthetic process or ganglioside in a ratio of less than 1:2, less than 1:3, less than 1:4, less than 1:5, less than 1:8, less than 1:10, less than 1:15, less than 1:20, less than 1:25, less than 1:30, less than 1:40, less than 1:50, less than 1:100, when compared to the inhibition of a late stage biosynthetic process or ganglioside.

In certain instances, limiting modifications to glycans limits undesirable or toxic side effects. In some instances, the restricted distribution of the potential toxic effects makes toxicities more predictable as well. Therefore, in some instances, restriction of ganglioside synthesis modulators (e.g., inhibitors or promoters) to subsets of glycans, restrict side effects and makes identification, isolation and tracking the effects of the inhibitors more reliable. Similarly, in some instances, these effects make dose determination more reliable.

In some embodiments, ganglioside synthesis modulators (e.g., inhibitors or promoters) described herein target for modification (inhibition, alter, increase) of formation (structure and quantity) of a glycan (carbohydrate portion of a molecule) but not protein, not nucleic acid, not lipid. In some embodiments, ganglioside synthesis modulators (e.g., inhibitors or promoters) that target glycosphingolipids (GSL) provide effective therapies for CNS disorders and lysosomal storage diseases (Salidosis (GM3 accumulation), Tay Sachs and Sandhoff Diseases (both predominantly GM2 accumulation), GM1 gangliosidosis (GM1 accumulation)). In certain embodiments, ganglioside synthesis modulators (e.g., inhibitors or promoters) that inhibit Lactosylceramide Synthase (beta GalT1) or alpha 1,4 galactosyltransferase (GB3 synthase) treat Fabry disease (primarily GB3 accumulation). In some embodiments, ganglioside synthesis modulators (e.g., inhibitors or promoters) that inhibit Lactosylceramide Synthase (beta GalT1) or ST8Sial-I/ST-II (GD3 synthase) treat cancer.

Glycosphingolipids are glycolipids built on a ceramide lipid moiety consisting of a long chain amino alcohol (sphingosine) in amid linkage to a fatty acid. In some instances, the first sugars linked to the C-1 hydroxyl group of ceramide are either β-linked Gal (GalCer) or Glu (GluCer). In certain instances, GalCer is a major glycan in the brain with essential roles in the structure and function of myelin. GlcCer is abundant in certain tissues. In skin, GlcCer and its derivatives, have important functions in the formation of the water barrier. In more complex vertebrate glycosphingolipids, the glucose moiety is often substituted with β-linked galactose on the C-4 hydroxyl of glucose to give lactosylceramide (Gal β1-4Glc βCer). In certain instances, further extensions of the glycan give a series of neutral core structures that form the basis of the nomenclature of glycosphingolipids. In certain instances, ganglio-series of glycosphingolipids are based on the neutral core structure Gal β1-3GalNAc β1-4Gal β1-4Glc βCer. In mammals the ganglio series of glycosphingolipids are broadly distributed but predominate in the brain. In various instances, all sialylated glycosphingolipids are known as gangliosides regardless of whether they are based on the ganglio-series neutral core structure.

In some embodiments, ganglioside synthesis modulators (e.g., inhibitors or promoters) described herein specifically modulate (e.g., inhibit or promote) gangliosides characterized by one or more of the following:

a. Glycans containing glucose (Glu)

b. Glycans containing galactose (Gal)

c. Glycans containing N-acetylglucosamine (GlcNAc)

d. Glycans containing N-acetylgalactosamine (GalNAc)

e. Glycans containing mannose (Man)

f. Glycans containing xylose (Xyl)

g. Glycans containing fucose (Fuc)

h. Glycans containing sialic acid (Sia)

i. Glycans with the structure Gal(β 1-4)GlcβCer, LacCer

j. Glycans in the Ganglio series

k. Glycan structures in the O-ganglioside series
i. GalNAc (β 1-4) Gal(β 1-4)GlcβCer, GA₂
ii. Gal(β 1-3) GalNAc (β 1-4) Gal(β 1-4)GlcβCer, GA1
iii. Gal(β 1-3) [Sia(α2-6)] GalNAc (β 1-4) Gal(β 1-4)GlcβCer, GM1α
iv. Gal(β 1-3) [Sia(α2-8)Sia(α2-6)]GalNAc (β 1-4) Gal(β 1-4)GlcβCer, GD1β
v. Sia(α1-3)Gal(β 1-3) GalNAc (β 1-4) Gal(β 1-4)GlcβCer, cisGM1 (GM1b)
vi. Sia(α2-8)[Sia(α2-3)]Gal(β 1-3) GalNAc (β 1-4) Gal(β 1-4)GlcβCer, GD1 (GD1c)
vii. Sia(α2-3)Gal(β 1-3) [Sia(α2-6)] GalNAc (β 1-4) Gal(β 1-4)GlcβCer, GD1 α

l. Glycan structures in the A-ganglioside series
i. Sia(α2-3)Gal(β 1-4)GlcβCer, GM3
ii. GalNAc (β 1-4) [Sia(α2-3)]Gal(β 1-4)GlcβCer, GM2
iii. Gal(β 1-3) GalNAc (β 1-4) [Sia(α2-3)]Gal(β 1-4)GlcβCer, GM1 (GM1a)
iv. Sia(α2-3)Gal(β 1-3) GalNAc (β 1-4) [Sia(α2-3)]Gal(β 1-4)GlcβCer, GD1a
v. Sia(α2-8)Sia(α2-3)Gal(β 1-3)[GalNAc (β 1-4) [Sia(α2-3)]Gal(β 1-4)GlcβCer, GT1a
vi. Sia(α2-3)Gal(β 1-3) [Sia(α2-6)] GalNAc (β 1-4) [Sia(α2-3)]Gal(β 1-4)GlcβCer, GT1a α

m. Glycan structures in the B-ganglioside series
i. Sia(α2-8)Sia(α2-3)Gal(β 1-4)GlcβCer, GD3
ii. GalNAc (β 1-4)[Sia(α2-8)Sia(α2-3)]Gal(β 1-4)GlcβCer, GD2
iii. Gal(β 1-3)GalNAc (β 1-4)[Sia(α2-8)Sia(α2-3)]Gal(β 1-4)GlcβCer, GD1b
iv. Sia(α2-3)Gal(β 1-3)GalNAc (β 1-4)[Sia(α2-8)Sia(α2-3)]Gal(β 1-4)GlcβCer, GT1b
v. Sia(α2-8)Sia(α2-3)Gal(β 1-3)GalNAc (β 1-4)[Sia(α2-8)Sia(α2-3)]Gal(β 1-4)GlcβCer, GQ1b
vi. Sia(α2-3)Gal(β 1-3) [Sia(α2-6)] GalNAc (β 1-4)[Sia(α2-8)Sia(α2-3)]Gal(β 1-4)GlcβCer, GQ1bα

n. Glycan structures in the C-ganglioside series
i. Sia(α2-8)Sia(α2-8)Sia(α2-3)Gal(β 1-4)GlcβCer, GT3
ii. GalNAc (β 1-4)[Sia(α2-8)Sia(α2-8)Sia](α2-3)Gal(β 1-4)GlcβCer, GT2
iii. Gal(β 1-3)GalNAc (β 1-4)[Sia(α2-8)Sia(α2-8)Sia](α2-3)Gal(β 1-4)GlcβCer, GT1c
iv. Sia(α2-3)Gal(β 1-3)GalNAc (β 1-4)[Sia(α2-8)Sia(α2-8)Sia](α2-3)Gal(β 1-4)GlcβCer, GQ1c
v. Sia(α2-8)Sia(α2-3)Gal(β 1-3)GalNAc (β 1-4)[Sia(α2-8)Sia(α2-8)Sia](α2-3)Gal(β 1-4)GlcβCer, GP1c.

For example, in some embodiments, inhibitors of lactoceramide synthase or other further downstream biosynthetic enzymes have the advantage of not affecting glucoceramide levels, and would, in turn, lead to specificity of ganglioside inhibition. In certain embodiments, a more specific inhibitor directed at blocking the biosynthesis of only the ganglioside subset of GSLs reduces unwanted side effects due to the inhibition of all GSLs. In some embodiments, inhibitors of GM3 synthase (ST3Gal-V), GM2/GD2 synthase (b1-4 GalNAc transferase), GD3 synthase (ST8Sial-I), Gal TII, ST3Gal-II or downstream enzymes affect only the ganglioside family.

In certain embodiments, selectivity of ganglioside synthesis modulators (e.g., inhibitors) is beneficial in order to target specific disorders without adversely impacting properly functioning glycan biosynthetic processes. In some embodiments, therapeutic methods utilizing selective ganglioside synthesis modulators (e.g., inhibitors) have improved toxicity profiles compared to non-selective glycan synthesis inhibitors. In some embodiments, selective ganglioside synthesis modulators (e.g., inhibitors) modulate (e.g., inhibit or promote) late stage processes (including, e.g., enzyme activity involved in the ganglioside preparation/synthetic pathway, enzyme activity involved in the ganglioside degradation pathway, other enzyme activity that affects the character of ganglioside, or the like) in the ganglioside biosynthetic pathway.

In some embodiments, modulation of ganglioside biosynthesis includes the modulation of or is selective for a specific ganglioside synthase. In some embodiments, a ganglioside synthase is a glucosyl ceramide synthase (GlcCer synthase). In some embodiments, a ganglioside synthase is a galactosyl ceramide synthase (GalCer synthase). In specific embodiments, the ganglioside synthase is a lactosyl ceramide synthase (LacCer synthase). In more specific embodiments, the synthase is a LacCer synthase, as compared to a GalCer synthase or a GlcCer synthase. In certain instances, specificity includes inhibition of the indicated type of synthase by a ratio of greater than about 10:1, greater than about 9:1, greater than about 8:1, greater than about 7:1, greater than about 6:1, greater than about 5:1, greater than about 4:1, greater than about 3:1, or greater than about 2:1 over the other types of synthases.

In some embodiments, modulation of ganglioside biosynthesis includes the modulation of or is selective for a specific ganglioside glycosyltransferase. In some embodiments, a ganglioside glycosyltransferase is a glucosyl transferase. In some embodiments, a ganglioside glycosyltransferase is a galactosyl transferase. In some embodiments, a ganglioside glycosyltransferase is a sialyl transferase. In more specific embodiments, the glycosyltransferase is a GT_{1b} synthase including, a GM₃ synthase (e.g., sialyl transferase I), a GM₂/GD₂ synthase (e.g., β1-4 GalNAc transferase), a GD₃ synthase (e.g. sialyl transferase II), Gal TII, or Sialyl TIV, as compared to a GM₁ synthase, a GD₁ synthase, a GT_{1aα} synthase, or a GT_{1c} synthase. In certain instances, specificity includes inhibition of the indicated type of glycosyltransferase by a ratio of greater than about 10:1, greater than about 9:1, greater than about 8:1, greater than about 7:1, greater than about 6:1, greater than about 5:1, greater than about 4:1, greater than about 3:1, or greater than about 2:1 over the other types of glycosyltransferases.

In yet a further embodiment ganglioside biosynthesis inhibitors includes the inhibitors of the addition of a NeuNAc residue to a ganglioside having the structure:

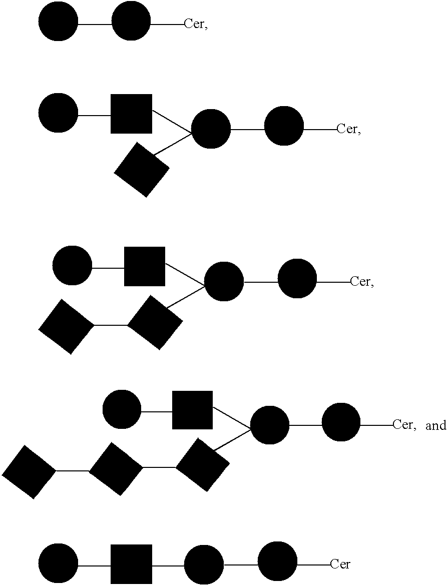

via an α2,3 linkage
wherein: [symbol]
is a galactose residue; [symbol]
is a glucose residue; [symbol]
is an N-acetylgalactosamine residue; [symbol]
is a NeuNAc residue; and
Cer is ceramide

In one embodiment the selective inhibitor of ganglioside biosynthesis inhibits the addition of a NeuNAc residue to a ganglioside having the structure:

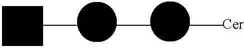

via an α2,3 linkage;
wherein: [symbol]
is a galactose residue; [symbol]
is a glucose residue; [symbol]
is an N-acetylgalactosamine residue; and
Cer is ceramide

In another embodiment the selective inhibitor of ganglioside biosynthesis inhibits the addition of a NeuNAc residue to a ganglioside having the structure:

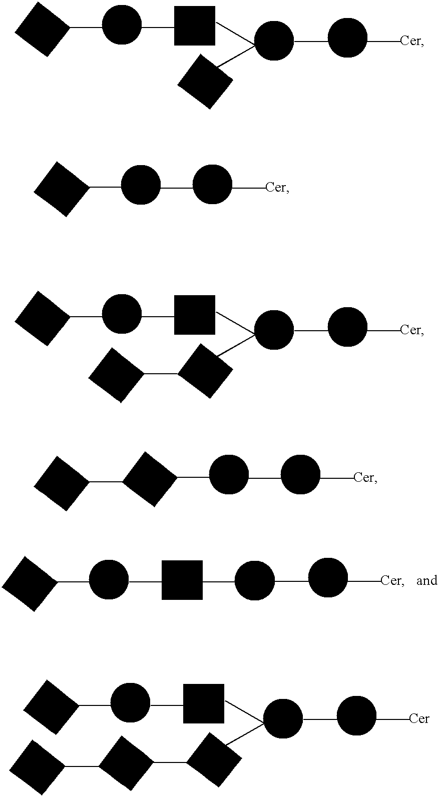

via an α2,8 linkage;
wherein: [symbol]
is a galactose residue; [symbol]
is a glucose residue; [symbol]
is an N-acetylgalactosamine residue; [symbol]
is a NeuNAc residue; and
Cer is ceramide

In yet another embodiment the selective inhibitor of ganglioside biosynthesis inhibits the addition of an N-acetylgalactosamine residue to a ganglioside having the structure:

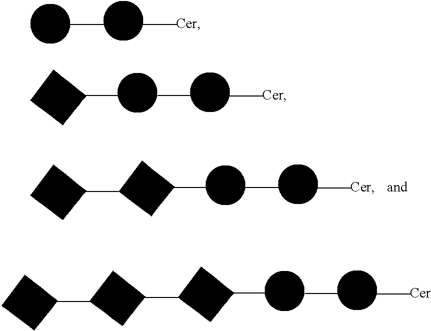

via an β1,4 linkage;
wherein: [symbol]
is a galactose residue; [symbol]
is a glucose residue; [symbol]
is an N-acetylgalactosamine residue; [symbol]
is a NeuNAc residue; and
Cer is ceramide

In a further embodiment the selective inhibitor of ganglioside biosynthesis inhibits the addition of a galactose residue to a ganglioside having the structure:

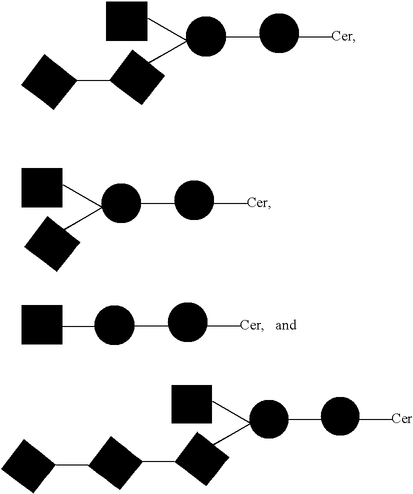

via an β1,3 linkage;
wherein: [symbol]
is a galactose residue; [symbol]
is a glucose residue; [symbol]
is an N-acetylgalactosamine residue; [symbol]
is a NeuNAc residue; and
Cer is ceramide

In some instances, the modulation of ganglioside biosynthesis includes the modulation of the oxygen sulfation (i.e., sulfation of the hydroxy group used interchangeably herein with O-sulfation), N-sulfation, O-acetylation, N-acetylation, O-acetylation, phosphorylation or a combination thereof. In some embodiments, a ganglioside synthesis inhibitor modulates one or more sulfotransferase. In some embodiments, modulation of O-sulfation includes the inhibition of the 3-O sulfation of a galactosyl residue of the ceramide-linked glycan. In certain instances, specificity includes inhibition, modulation or promotion of the indicated type of sulfation by a ratio of greater than about 10:1, greater than about 9:1, greater than about 8:1, greater than about 7:1, greater than about 6:1, greater than about 5:1, greater than about 4:1, greater than about 3:1, or greater than about 2:1 over the other types of sulfation.

In some instances, the modulation of ganglioside biosynthesis includes the modulation of the synthesis of 0 series gangliosides (e.g., GD_{1c}, GM_{1b}, GA₁ or GA₂ ganglioside), A series gangliosides (e.g., GT₁ₐ, GD₁ₐ, GM₂, GM₁ₐ or GM₃, ganglioside), B series gangliosides (e.g., GQ_{1b}, GT_{1b}, GD_{1b}, GD₂ or GD₃ ganglioside) or C series gangliosides (e.g., GP_{1c}, GQ_{1c}, GT_{1c}, GT₂ or GT₃ ganglioside). In certain instances, specificity includes inhibition, modulation or promotion of the synthesis of the indicated type of ganglioside by a ratio of greater than about 10:1, greater than about 9:1, greater than about 8:1, greater than about 7:1, greater than about 6:1, greater than about 5:1, greater than about 4:1, greater than about 3:1, or greater than about 2:1 over the other types of gangliosides and/or other types of glycans. For example, in certain instances, specificity includes inhibition, modulation or promotion of the synthesis of GM₁ₐ gangliosides and/or GT_{1b} gangliosides by a ratio of greater than about 10:1, greater than about 9:1, greater than about 8:1, greater than about 7:1, greater than about 6:1, greater than about 5:1, greater than about 4:1, greater than about 3:1, or greater than about 2:1 over the other types of gangliosides and/or other glycans.

In certain embodiments, ganglioside synthesis inhibitors or modulators of ganglioside biosynthesis are compounds that modify the nature (e.g., character, structure and/or concentration) of ganglioside on a cellular surface (including vesicles), cell, tissue, organ or individual when contacted or administered to the cell, tissue, organ or individual. It is to be understood that contacting a cell, tissue, or organ is possible via the administration to an individual within whom such cell, tissue or organ resides. In certain instances, ganglioside synthesis inhibitors or modulators of ganglioside biosynthesis modify the character and/or concentration of ganglioside in a targeted type of cell, tissue type or organ. In other instances, ganglioside synthesis inhibitors or modulators of ganglioside biosynthesis modify the character and/or concentration of gangliosides in a systemic manner.

In certain embodiments, a ganglioside synthesis inhibitor (used interchangeably herein with a modulator of ganglioside biosynthesis) alters or disrupts the nature of ganglioside compared to endogenous ganglioside in an amount sufficient to alter or disrupt ganglioside binding, ganglioside signaling, or a combination thereof. In some embodiments, the ganglioside synthesis inhibitor alters or disrupts the nature of ganglioside in a selected tissue type or organ compared to endogenous ganglioside in the selected tissue type or organ. In some embodiments, the selected tissue is, by way of non-limiting example, brain tissue, liver tissue, kidney tissue, intestinal tissue, blood, skin tissue, or the like. In some embodiments, a ganglioside synthesis inhibitor as described herein alters or disrupts the nature of ganglioside compared to endogenous ganglioside by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 35%, at least about 40%, or more.

In certain embodiments, the ganglioside synthesis inhibitor described herein alters or disrupts the concentration of ganglioside compared to endogenous ganglioside in a cell, tissue, organ, or individual by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 35%, at least about 40%, or more. In certain embodiments, the ganglioside synthesis inhibitor described herein alters or disrupts the synthesis of GT_{1b} gangliosides compared to endogenous gangliosides in a cell, tissue, organ, or individual by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 35%, at least about 40%, or more.

In certain embodiments, the ganglioside synthesis inhibitor described herein alters or disrupts the chain length (or ganglioside molecular weight) of ganglioside compared to endogenous ganglioside in a cell, tissue, organ, or individual by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 35%, at least about 40%, or more. In certain embodiments, the ganglioside synthesis inhibitor described herein alters or disrupts, in combination (e.g., the sum of the change in amount, sulfation, concentration, sialylation and/or chain length), the nature of ganglioside compared to endogenous ganglioside in a cell, tissue, organ, or individual by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 35%, at least about 40%, or more. In certain embodiments, a ganglioside synthesis inhibitor as described herein alters or disrupts the sulfation and/or phosphorylation of a linkage region of ganglioside compared to endogenous ganglioside in an organism, organ, tissue or cell by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 35%, at least about 40%, or more.

In certain embodiments, a ganglioside synthesis inhibitor as described herein modifies, alters or disrupts the amount of gangliosides on a cell, tissue, organ or individual compared to amounts of endogenous ganglioside in an organism, organ, tissue or cell by at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 35%, at least about 40%, at least about 50%, at least about 60%, at least about 70% or more. As used herein, endogenous ganglioside is described as ganglioside present in the absence of treatment or contact with a ganglioside synthesis inhibitor.

In some embodiments, a modified, altered or disrupted ganglioside contains less than about 5%, less than about 10%, less than 15%, less than about 20%, less than about 30%, less than about 40%, less than about 50%, less than about 60%, less than about 70%, less than about 80%, less than about 90%, less than about 95%, less than about 98%, or less than about 99% of one or more of any specific type of ganglioside(s) (e.g., O series, B series, A series or C series gangliosides (e.g., GT_{1b}, GM₁ₐ, GM₂, GM₃, GD₃, GT_{1c}, GA₂, GA₁, GM_{1b}, GD_{1c}, GD₁ₐ, GT₁ₐ, GD₂, GD_{1b}, GQ₁b, GT₃, GT₂, GT₁ₐ, GQ_{1c}, or GP_{1c} gangliosides or the like)) compared to a ganglioside that has not been modified, disrupted or altered. By way of example, in some embodiments, a modified, altered or disrupted ganglioside contains less than about 20%, less than about 30%, less than about 40%, less than about 50%, less than about 60%, less than about 70% or less than about 80% of GM3 gangliosides, or less than about 20%, less than about 30%, less than about 40%, less than about 50%, less than about 60%, less than about 70% or less than about 80% of GD3 gangliosides, or a combination thereof, compared to a ganglioside that has not been modified, disrupted or altered. Moreover, it is to be understood that such glycolipids, e.g., as synthesized in the presence of a glycolipid biosynthesis modulator provided herein and/or as described above, are provided for in various embodiments herein.

In some embodiments, the comparison between altered or disrupted ganglioside compared to endogenous ganglioside is based on the average characteristic (e.g., the concentration, 3-O sulfation, sialylation, chain length or molecular weight, combinations thereof, or the like) of the altered or disrupted ganglioside. Furthermore, in some embodiments, the comparison between altered or disrupted ganglioside is based on a comparison of the modified O, A, B or C gangliosides (e.g., GT_{1b} domains of a modified B ganglioside) to O, A, B or C endogenous gangliosides (e.g. GT_{1b} domains of endogenous B gangliosides). In some instances, the degree or nature of GT_{1b} in the domains that have high GT_{1b} in endogenous ganglioside are increased or decreased in the modified ganglioside. In certain instances, the degree or nature of sialylation in the domains that have low sialylation in endogenous ganglioside have increased sialylation in the modified ganglioside. In some embodiments, the concentration, amount, character, and/or structure of ganglioside is determined in any suitable manner, including those set forth herein. As used herein, altering includes increasing or decreasing. Furthermore, as used herein, disrupting includes reducing or inhibiting.

In some embodiments, the ganglioside synthesis inhibitor described herein alters or disrupts the nature of the ganglioside such that it inhibits ganglioside signaling. In other specific embodiments, the ganglioside synthesis inhibitor described herein alters or disrupts the nature of the ganglioside such that it inhibits ganglioside binding. In more specific embodiments, the ganglioside synthesis inhibitor described herein alters or disrupts the nature of the ganglioside such that it inhibits ganglioside binding and ganglioside signaling. In some embodiments, the ganglioside synthesis inhibitor alters or disrupts the nature of the ganglioside such that it inhibits the binding, signaling, or a combination thereof of any lectin (including polypeptides) subject to ganglioside binding, signaling or a combination thereof, in the absence of a ganglioside synthesis inhibitor. In some embodiments, the polypeptide is, by way of non-limiting example, a cell adhesion molecule (CAM). In certain embodiments, the CAM is an exogenous CAM, e.g., a bacterial lectin. In certain embodiments, the CAM is an endogenous CAM and includes, by way of non-limiting examples, E-selectin, L-selectin or P-selectin.

In some embodiments, a ganglioside synthesis inhibitor is an agent that when contacted or administered to a human liver cell, a human liver tissue, a human liver, or a human results in an average number of GT_{1b} gangliosides of less than about 1.2% (w/w), less than about 1.1% (w/w), less than about 1.0% (w/w), less than about 0.9% (w/w), less than about 0.8% (w/w), less than about 0.7% (w/w), less than about 0.6% (w/w), or less than about 0.5% (w/w) in the liver cell, liver tissue, the liver, or the liver of the human, respectively. In some embodiments, a ganglioside synthesis inhibitor is an agent that when contacted or administered to a pig liver cell, pig liver tissue, a pig liver, or a pig results in an average number of GT_{1b} gangliosides of less than about 1.0% (w/w), less than about 0.9% (w/w), less than about 0.8% (w/w), less than about 0.7% (w/w), less than about 0.6% (w/w), or less than about 0.5% (w/w) in the liver cell, liver tissue, the liver, or the liver of the pig, respectively. In some embodiments, a ganglioside synthesis inhibitor is an agent that when contacted or administered to a mouse liver cell, mouse liver tissue, a mouse liver, or a mouse results in an average number of GT_{1b} gangliosides of less than about 0.9% (w/w), less than about 0.8% (w/w), less than about 0.7% (w/w), less than about 0.6% (w/w), less than about 0.5% (w/w), less than about 0.4% (w/w), or less than about 0.3% (w/w) in the liver cell, liver tissue, the liver, or the liver of the mouse, respectively. As used herein, altering includes increasing or decreasing. Furthermore, as used herein, disrupting includes reducing or inhibiting.

In some embodiments, a ganglioside synthesis inhibitor is an agent that when contacted or administered to a human liver cell, a human liver tissue, a human liver, or a human results in an average number of GT_{1b} gangliosides of less than about 1.2 mol. %, less than about 1.1 mol. %, less than about 1.0 mol. %, less than about 0.9 mol. %, less than about 0.8 mol. %, less than about 0.7 mol. %, less than about 0.6 mol. %, or less than about 0.5 mol. % in the liver cell, liver tissue, the liver, or the liver of the human, respectively. Furthermore, as used herein, mol. % is the molar percentage of the selected ganglioside component compared to the total number of ganglioside components in the ganglioside(s) present and/or analyzed. In some embodiments, a ganglioside synthesis inhibitor is an agent that when contacted or administered to a human liver cell, a human liver tissue, a human liver, or a human results in an average number of GT_{1b} gangliosides of each glycan component of less than about 20 mol %, about 18 mol %. about 15 mol. %, less than about 14 mol. %, less than about 12 mol. %, less than about 10 mol. %, less than about 8 mol. %, less than about 7 mol. %, less than about 6 mol. %, less than about 5 mol. %, less than about 4 mol % or less than about 3 mol % in the liver cell, liver tissue, the liver, or the liver of the human, respectively. In some embodiments, a ganglioside synthesis inhibitor is an agent that when contacted or administered to a human liver cell, a human liver tissue, a human liver, or a human results in an average number of GT_{1b} gangliosides of each glycan component of less than about 0.7 mol. %, less than about 0.6 mol. %, less than about 0.5 mol. %, less than about 0.4 mol. %, or less than about 0.3 mol. % in the liver cell, liver tissue, the liver, or the liver of the human, respectively. In some embodiments, a ganglioside synthesis inhibitor described herein reduces the average number of GT_{1b} gangliosides in a cell, tissue, organ or individual. In certain embodiments, the amount of ganglioside synthesis inhibitor administered is an effective amount. In further embodiments, the effective amount is an amount having a minimal lethality. In more specific embodiments, the LD₅₀:ED₅₀ is greater than about 1.1, greater than about 1.2, greater than about 1.3, greater than about 1.4, greater than about 1.5, greater than about 2, greater than about 5, greater than about 10, or more. In some embodiments, a therapeutically effective amount is about 0.1 mg to about 10 g.

In some embodiments, a glycolipid biosynthesis modulator (e.g., inhibitor or promoter) described herein is a selective glycolipid synthesis modulator (e.g., inhibitor or promoter). In some embodiments, a selective glycolipid inhibitor selectively affects (e.g., alters or disrupts the nature, such as the concentration, chain length, average number of sialic acid residues, etc. of) a glycolipid (e.g. a LacCer glycolipid) or a specific type of glycolipid compared to other glycans and/or other glycolipids (e.g., GalCer glycolipid). In certain embodiments, the selective glycolipid synthesis modulator affects (e.g., inhibits or promotes) the biosynthesis of glycolipids (e.g., the glycan portion thereof), without affecting or significantly affecting the biosynthesis of proteoglycans (e.g., the glycan portion thereof). In various embodiments, the selective glycolipid biosynthesis inhibitor selectively modulates (e.g., inhibits or promotes) the synthesis of glycolipids over proteoglycans by a ratio of greater than about 2:1, about 3:1, about 4:1, about 5:1, about 6:1, about 8:1, about 10:1 or more. In some embodiments, the selective glycolipid synthesis modulator affects (e.g., inhibits or promotes) the biosynthesis of glycolipids compared to the biosynthesis of one or more of N-linked glycans, glycosaminoglycans (GAGs), O-linked glycans, or a combination thereof. In various embodiments, the selective glycolipid biosynthesis inhibitor selectively modulates (e.g., inhibits or promotes) the synthesis of glycolipids over N-linked glycans, glycosaminoglycans (GAGs), O-linked glycans, or a combination thereof by a ratio of greater than about 2:1, about 3:1, about 4:1, about 5:1, about 6:1, about 8:1, about 10:1 or more.

In specific embodiments, a selective glycolipid biosynthesis modulator described herein selectively affects (e.g., promotes or inhibits) a specific type or series of glycolipid compared to one or more other type or series of glycolipids. In some embodiments, a glycolipid biosynthesis modulator (e.g., inhibitor or promoter) is a selective ganglioside synthesis modulator (e.g., inhibitor or promoter). In certain embodiments, a selective glycolipid biosynthesis modulator (e.g., inhibitor or promoter) selectively affects (e.g., promotes or inhibits) the synthesis of a specific type or series of glycolipid (e.g., one or more of a lacto-series glycolipid, a lactoneo-series glycolipid, a globo-series glycolipid, a isoglobo-series glycolipid, a ganglio-series glycolipid, a muco-series glycolipid, a gala-series glycolipid, a sulfatide-series glycolipid, or a combination thereof) compared to the synthesis of one or more of another type or series of glycolipid (e.g., one or more of a lacto-series glycolipid, a lactoneo-series glycolipid, a globo-series glycolipid, a isoglobo-series glycolipid, a ganglio-series glycolipid, a muco-series glycolipid, a gala-series glycolipid, a sulfatide-series glycolipid, or a combination thereof). In certain embodiments, the selective glycolipid biosynthesis modulator (e.g., inhibitor or promoter) selectively modulates (e.g., inhibits or promotes) the synthesis of a first specific type or series of glycolipid compared to one or more different types or series of glycolipids by a ratio of greater than about 2:1, about 3:1, about 4:1, about 5:1, about 6:1, about 8:1, about 10:1 or more.

In certain embodiments, a selective glycolipid modulator (e.g., inhibitor or promoter) selective affects (e.g., inhibits or promotes) the activity of a specific enzyme involved in the biosynthesis of one or more glycolipid (e.g., one or more of a serine palmitoyltransferase, a 3-dehydrosphinganine reductase, a sphinganine N-acyltransferase, a dihydroceramide desaturase, a ceramide glucosyltransferase, a galactosylceramide synthase, a glucosylceramide synthase, a lactosylceramide synthase, a sialylα2-3 transferase (GM3 synthase), a GalNAcβ1-4 transferase, a lacto β1-3 GlcNAc transferase, a lacto β1-3 Gal transferase, a neolacto β1-4 Gal transferase, a globo α1-4 Gal transferase, a globo β1-3 GlcNAc transferase, an isoglobo α1-3 Gal transferase, an isoglobo β1-3 GlcNAc transferase, a muco β1-3 Gal transferase, a β3GlcA transferase, a β3GalNAc transferase, a sialyltransferase, a fucosyltransferase, a sulfotransferase, a B-blood Group transferase, a β3Gal transferase, a βGlcNAc transferase, a α-Gal transferase, an O-acetyltransferase, an A-blood Group transferase, sialyl transferase I, sialyl transferase II, sialyl transferase III, GalNAc transferase, Gal transferase II, sialyl transferase IV, sialyl transferase V, any other enzyme described herein as being involved in the biosynthesis of a glycolipid, or a combination thereof), while not affecting or not significantly affecting the activity of one or more or any other enzyme involved in the biosynthesis of one or more glycolipid (e.g., one or more of a serine palmitoyltransferase, a 3-dehydrosphinganine reductase, a sphinganine N-acyltransferase, a dihydroceramide desaturase, a ceramide glucosyltransferase, a galactosylceramide synthase, a glucosylceramide synthase, a lactosylceramide synthase, a sialylα2-3 transferase (GM3 synthase), a GalNAcβ1-4 transferase, a lacto β1-3 GlcNAc transferase, a lacto β1-3 Gal transferase, a neolacto β1-4 Gal transferase, a globo α1-4 Gal transferase, a globo β1-3 GlcNAc transferase, an isoglobo α1-3 Gal transferase, an isoglobo β1-3 GlcNAc transferase, a muco β1-3 Gal transferase, a βGlcA transferase, a βGalNAc transferase, a sialyltransferase, a fucosyltransferase, a sulfotransferase, a B-blood Group transferase, a βGal transferase, a βGlcNAc transferase, a α-Gal transferase, an O-acetyltransferase, an A-blood Group transferase, sialyl transferase I, sialyl transferase II, sialyl transferase III, GalNAc transferase, Gal transferase II, sialyl transferase IV, sialyl transferase V, any other enzyme described herein as being involved in the biosynthesis of a glycolipid, or a combination thereof). In certain embodiments, the selective glycolipid biosynthesis modulator (e.g., inhibitor or promoter) selectively modulates (e.g., inhibits or promotes) enzyme involved in the biosynthesis of one or more glycolipid compared to one or more different enzyme involved in the biosynthesis of one or more glycolipid by a ratio of greater than about 2:1, about 3:1, about 4:1, about 5:1, about 6:1, about 8:1, about 10:1 or more.

In certain embodiments, a selective glycolipid modulator is a selective ganglio-series glycolipid modulator, compared to other glycolipids and non-glycolipid glycans. In some embodiments, selective ganglio-series glycolipid modulators (e.g., inhibitors or promoters) are selective for one or more of A series ganglioside, B series ganglioside, C series ganglioside, O series ganglioside, or a combination thereof compared to a different of one or more of a A series ganglioside, B series ganglioside, C series ganglioside, O series ganglioside, or a combination thereof. In certain embodiments, selective ganglio-series glycolipid modulators (e.g., inhibitors or promoters) are selective for one or more of a specific type of A series ganglioside, B series ganglioside, C series ganglioside, or O series ganglioside (e.g., one or more of G_{A2}, G_{A1}, G_{D1c}, G_{M2}, G_{M3}, G_{M1a}, G_{D1a}, G_{T1a}, G_{D2}, G_{D3}, OAc-G_{D3}, G_{T1b}, OAc-G_{Q1b}, G_{Q1b}, OAc-G_{T1b}, G_{T2}, G_{T3}, OAc-G_{T2}, G_{T1c}, G_{Q1c}, G_{P1c}, or any other glycolipid described herein, or a combination thereof) compared to one or more of any of one or more different A series ganglioside, B series ganglioside, C series ganglioside, and/or O series ganglioside (e.g., one or more of G_{A2}, G_{A1}, G_{D1c}, G_{M2}, G_{M3}, G_{M1a}, G_{D1a}, G_{T1a}, G_{D2}, G_{D3}, OAc-G_{D3}, G_{T1b}, OAc-G_{Q1b}, G_{Q1b}, OAc-G_{T1b}, G_{T2}, G_{T3}, OAc-G_{T2}, G_{T1c}, G_{Q1c}, G_{P1c}, or any other glycolipid described herein, or a combination thereof). In certain embodiments, the selective ganglioside biosynthesis modulator (e.g., inhibitor or promoter) selectively modulates (e.g., inhibits or promotes) one or more of a specific type of A series ganglioside, B series ganglioside, C series ganglioside, or O series ganglioside compared to one or more different specific type of A series ganglioside, B series ganglioside, C series ganglioside, and/or O series ganglioside by a ratio of greater than about 2:1, about 3:1, about 4:1, about 5:1, about 6:1, about 8:1, about 10:1 or more.

In some embodiments, a selective glycolipid modulators selectively inhibits or promotes a specific or select characteristic of a glycolipid, e.g., the amount of glycolipid, the glycan-length of the glycolipid, the number of sialic acid residues of a glycolipid, N-acetylation, O-sulfation, O-acylation of galactose residues, O-acetylation of sialic acid residues, or the like, while leaving other characteristics of the glycolipid unaffected or significantly unaffected.

In some embodiments, a selective ganglioside inhibitor selectively alters or disrupts the nature of the ganglioside (e.g., the concentration, chain length, average number of sialic acid residues, etc. thereof) of a ganglioside compared to other glycolipids (e.g., one or more other type or series of glycolipid). In some embodiments, a selective inhibitor of ganglioside synthesis modulates the synthesis of B series gangliosides (e.g., a G_{T1b} ganglioside or the like). In some embodiments, a selective inhibitor of ganglioside synthesis selectively reduces or inhibits the synthesis of O-series gangliosides (e.g., G_{M1b} gangliosides, or the like) compared to other gangliosides. In some embodiments, a selective inhibitor of ganglioside synthesis selectively reduces or inhibits the synthesis of A-series gangliosides (e.g., G_{D1a} gangliosides, or the like) compared to other gangliosides. In some embodiments, a selective inhibitor of ganglioside synthesis selectively reduces or inhibits the synthesis of B-series gangliosides (e.g., G_{T1b} gangliosides or the like) compared to other gangliosides. In some embodiments, a selective inhibitor of ganglioside synthesis selectively reduces or inhibits the synthesis of C-series gangliosides (e.g., G_{Q1c} gangliosides, or the like) compared to other gangliosides. In some embodiments, the selective ganglioside synthesis inhibitor selectively alters or disrupts the nature (e.g., concentration, chain length, average number of GT_{1b} gangliosides, etc.) of a G_{T1b} ganglioside compared to other gangliosides. In some embodiments, the selective ganglioside synthesis inhibitor selectively affects the biosynthesis and/or degradation of G_{T1b} gangliosides compared to other gangliosides. In certain embodiments, selective ganglioside synthesis inhibitors selectively inhibit synthesis of GT_{1b} gangliosides compared to extracellular glycans by a ratio of greater than about 2:1, about 3:1, about 4:1, about 5:1, about 6:1, about 8:1, about 10:1 or more. In some embodiments, the selective ganglioside synthesis inhibitor selectively affects the biosynthesis of sialylated GT_{1b} gangliosides, but not non-sialylated GT_{1b} gangliosides or extracellular glycans. In certain embodiments, selective ganglioside synthesis inhibitors selectively inhibit sialylated GT_{1b} gangliosides compared to non-sialylated GT_{1b} gangliosides and extracellular glycans by a ratio of greater than about 2:1, about 3:1, about 4:1, about 5:1, about 6:1, about 8:1, about 10:1 or more. In some embodiments, selective ganglioside synthesis inhibitors selectively inhibit the biosynthesis of GT_{1b} gangliosides but not G_{D1} or G_{M1} gangliosides or extracellular glycans. In certain embodiments, selective ganglioside synthesis inhibitors selectively inhibit GT_{1b} gangliosides compared to G_{D1} or G_{M1} gangliosides and extracellular glycans by a ratio of greater than about 2:1, about 3:1, about 4:1, about 5:1, about 6:1, about 8:1, about 10:1 or more. In some embodiments, selective GT_{1b} ganglioside synthesis inhibitors selectively inhibit GT_{1b} ganglioside, but not other gangliosides (e.g., other ceramide-linked glycans and extracellular glycans). In certain embodiments, selective GT_{1b} ganglioside synthesis inhibitors selectively inhibit GT_{1b} ganglioside compared to other ceramide-linked glycans and extracellular glycans by a ratio of greater than about 2:1, about 3:1, about 4:1, about 5:1, about 6:1, about 8:1, about 10:1 or more.

Furthermore, in certain embodiments, ganglioside synthesis inhibitors selectively modulate specific types of action that inhibit ganglioside function. For example, in some embodiments, ganglioside synthesis inhibitors selectively modulate sulfation, glycosylation, phosphorylation, sialylation, and/or degradation.

In some embodiments, ganglioside biosynthesis inhibitors selectively modulate a specific ganglioside glycosyltransferase. In some embodiments, a ganglioside glycosyltransferase is a glucosyl transferase. In some embodiments, a ganglioside glycosyltransferase is a galactosyl transferase. In some embodiments, a ganglioside glycosyltransferase is a sialyl transferase. In more specific embodiments, the glycosyltransferase is a GT_{1b} synthase including, a GM₃ synthase (e.g., sialyl transferase I), a GM₂/GD₂ synthase (e.g., β1-4 GalNAc transferase), a GD₃ synthase (e.g. sialyl transferase II), Gal TII, or Sialyl TIV, as compared to a GM₁ synthase, a GD₁ synthase, a GT_{1aα} synthase, or a GT_{1c} synthase. In some embodiments, ganglioside biosynthesis inhibitors selectively modulate a specific sulfotransferase (e.g., a 3-OH sulfotransferase).

In some embodiments, certain ganglioside synthesis inhibitors selectively modulate (e.g., promote or inhibit) glycosyltransferase, and/or specific types of glycosyltransferases. In some embodiments, ganglioside synthesis inhibitors selectively modulate (e.g., promote or inhibit) a GT_{1b} synthase, including, a GM₃ synthase (e.g., sialyl transferase I), a GM₂/GD₂ synthase (e.g., β1-4 GalNAc transferase), a GD₃ synthase (e.g. sialyl transferase II), Gal TII, or Sialyl TIV. In more specific embodiments, ganglioside synthesis inhibitors selectively modulate (e.g., promote or inhibit) one of β-galactoside α-2,3-sialyltransferase (ST3), α-N-acetyl-neuraminide α-2,8-sialyltransferase 1 (ST8), (α-N-acetyl-neuraminyl-2,3-beta-galactosyl-1,3)-N-acetylgalactosaminide α-2,6-sialyltransferase 3 (ST6), (α-N-acetyl-neuraminyl-2,3-beta-galactosyl-1,3)-N-acetylgalactosaminide α-2,6-sialyltransferase 4 (ST6), (α-N-acetyl-neuraminyl-2,3-β-galactosyl-1,3)-N-acetylgalactosaminide α-2,6-sialyltransferase 5 (ST6), (α-N-acetyl-neuraminyl-2,3-β-galactosyl-1,3)-N-acetylgalactosaminide α-2,6-sialyltransferase 6 (ST6), β-1,4-N-acetyl-galactosaminyl transferase 1, or UDP-Gal:βGlcNAc β1,3-galactosyltransferase, polypeptide 4.

In certain instances, targeting early biosynthetic enzymes upstream of UDP-Gal:GlcCer β 1-4 galactosyl transferase to make lactosylceramide (from GlcCer) may affect GalCer and have global effects. In some embodiments, modulating (e.g., inhibiting) enzymes downstream from LacCer would be restricted to gangliosides limiting potential side effects due to inhibition of the other GSL series. Therefore, in some embodiments, provided herein are ganglioside synthesis modulators (e.g., inhibitors or promoters) that selectively inhibit late stage ganglioside biosynthesis. In certain instances, late stage biosynthesis refers to structures beyond GlcCer. Late in biosynthesis includes lactosylceramide and the enzyme that forms it from GlcCer (lactosylceramide synthase) and beyond in the biosynthetic pathway (see FIG. 4).

In some embodiments, the ganglioside synthesis inhibitors are selective for gangliosides over other glycan classes. In some embodiments, the ganglioside synthesis inhibitors inhibit ganglioside synthesis in cells. In some embodiments, the ganglioside synthesis inhibitors are non-carbohydrate small molecules. In some embodiments, the ganglioside synthesis inhibitors inhibit ganglioside specific enzymes. In some embodiments, the ganglioside synthesis inhibitors inhibit enzymes that are downstream of enzymes that synthesize glycan molecules other than gangliosides. In some embodiments, the ganglioside synthesis inhibitors do not affect the synthesis of glycan molecules other than gangliosides. In some embodiments, the ganglioside synthesis inhibitors do not substantially affect the synthesis of glycan molecules other than gangliosides. In some embodiments, the ganglioside synthesis inhibitors inhibit enzymes in the endoplasmic reticulum and/or the Golgi apparatus. In some embodiments, the ganglioside synthesis inhibitors may not inhibit enzymes in the cytoplasm. In some embodiments, the ganglioside synthesis inhibitors may not substantially inhibit enzymes in the cytoplasm.

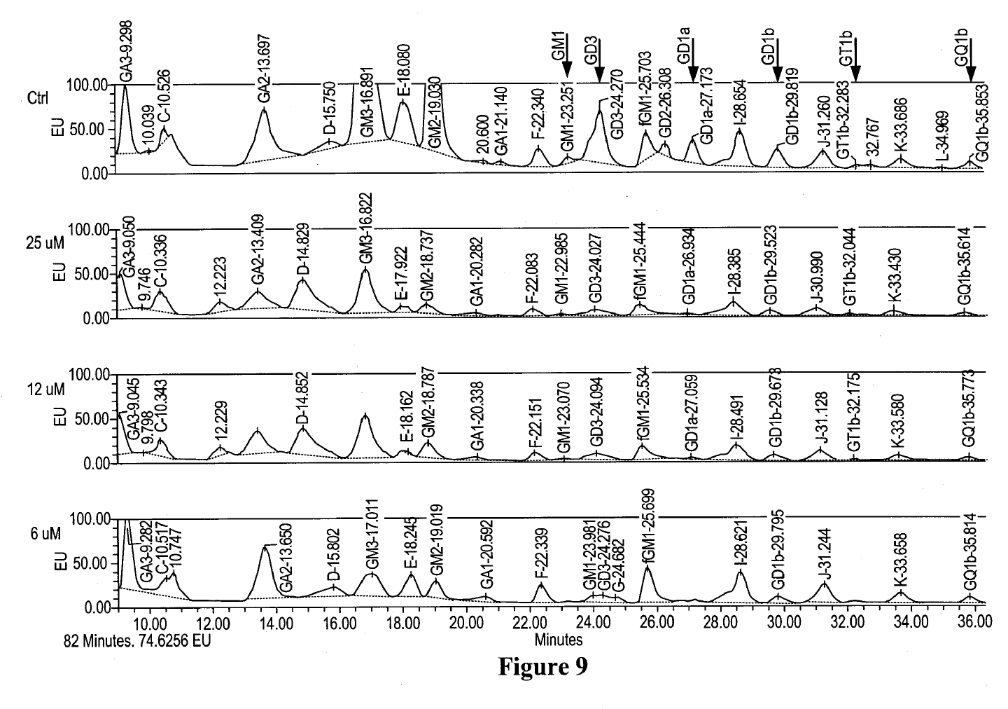

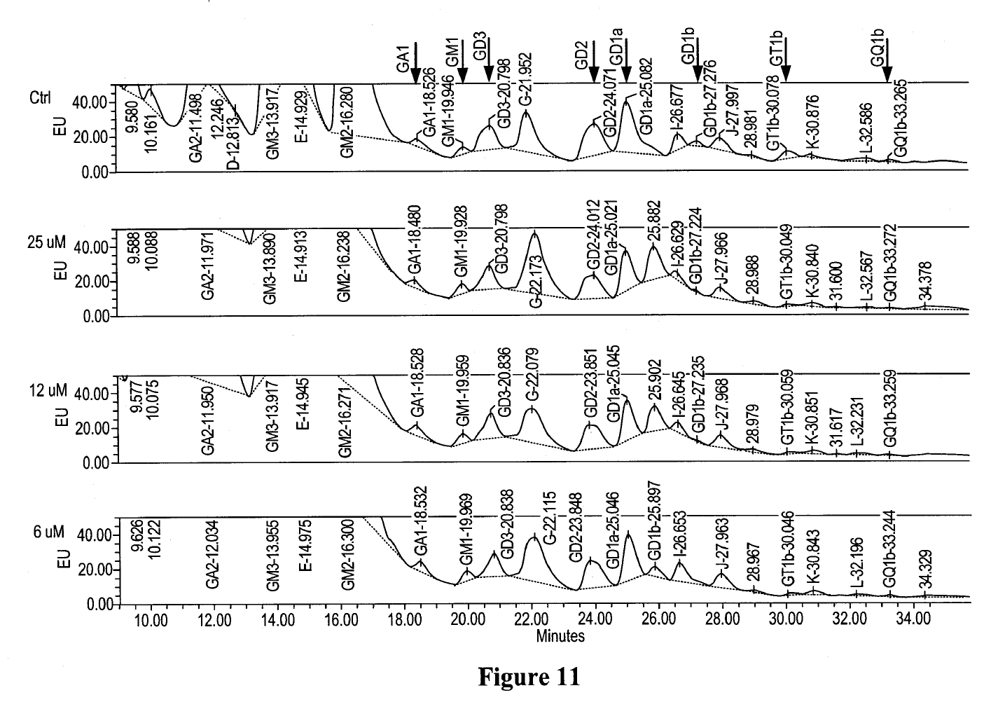

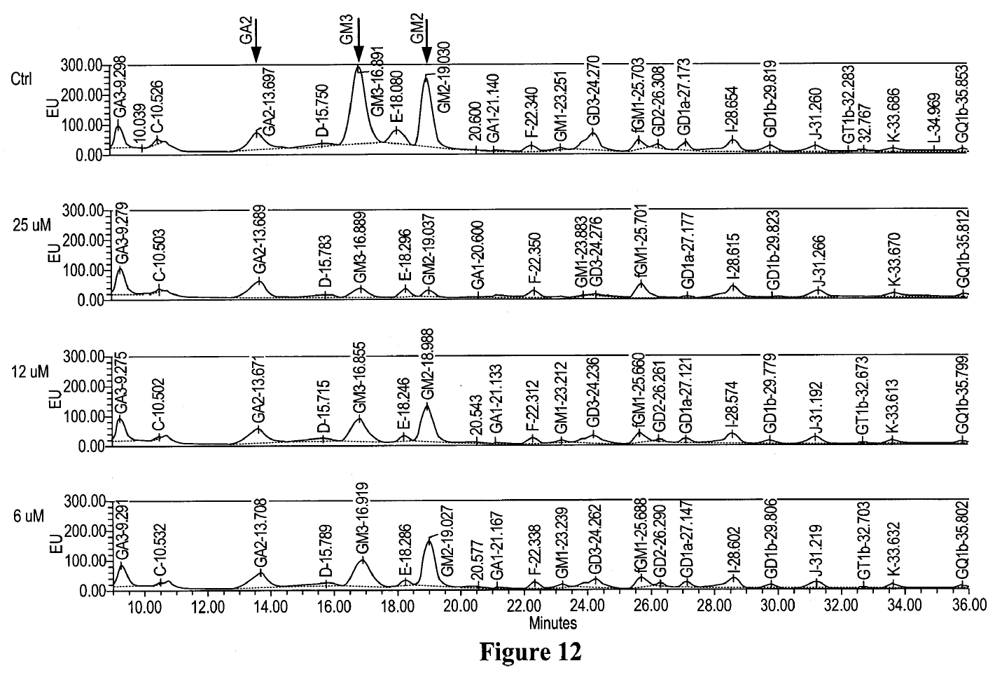

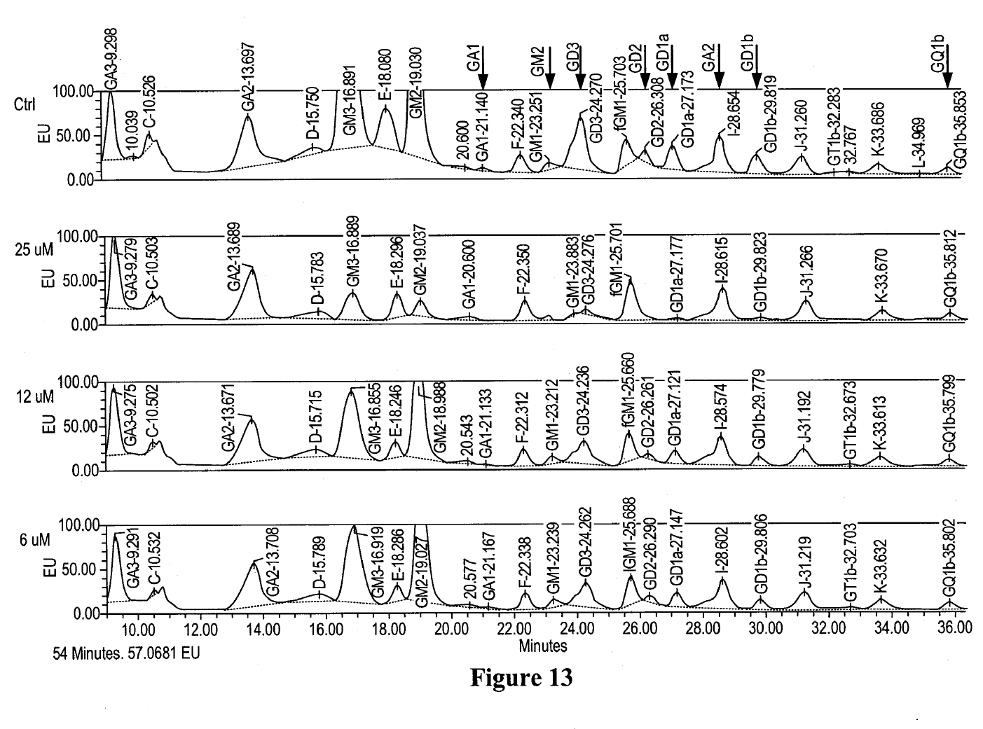

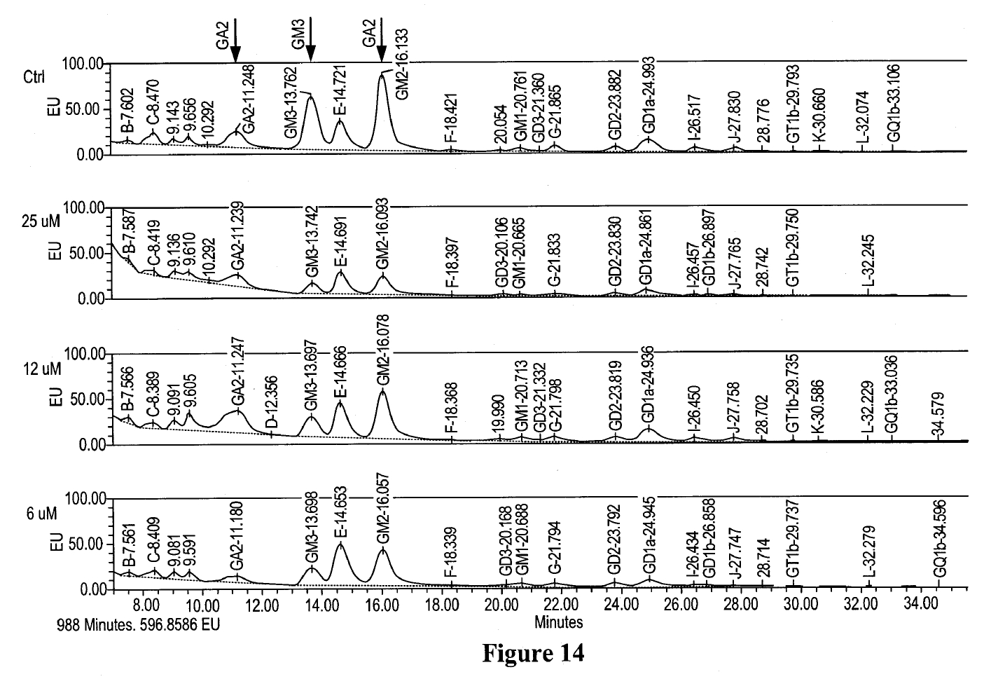

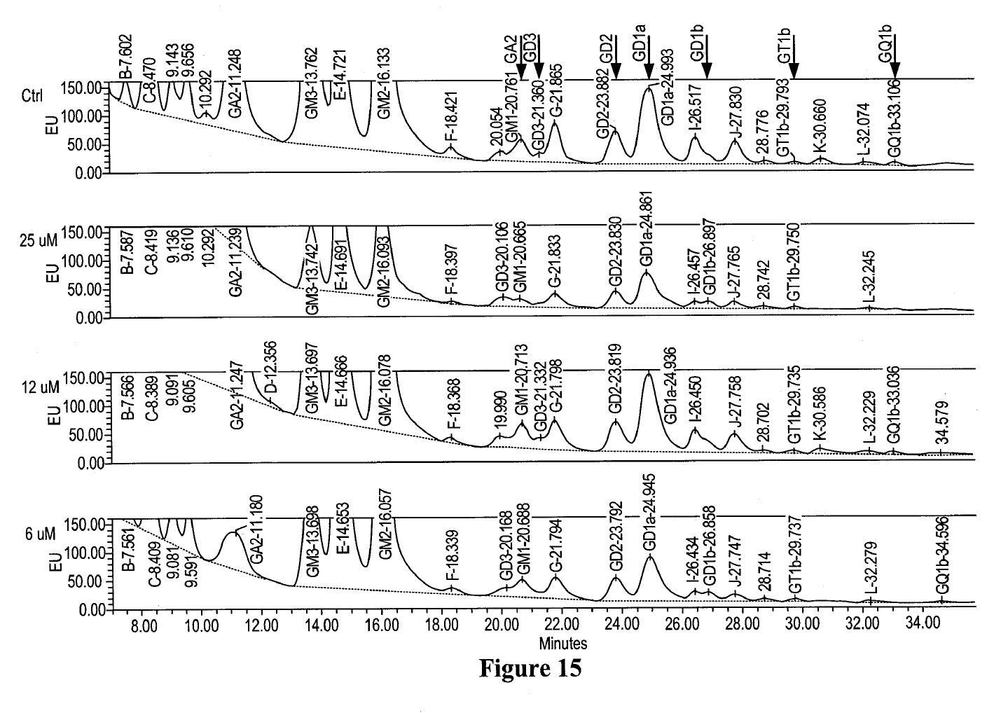

FIGS. 9-15 illustrate that in certain embodiments, ganglioside synthesis modulators (e.g., inhibitors or promoters) affect the synthesis of specific gangliosides.

In some embodiments, a ganglioside synthesis modulator (e.g., inhibitor or promoter) described herein is a selective ganglioside synthesis modulator (e.g., inhibitor or promoter) that modulates (e.g., inhibits) any specific transferase (or other enzyme) described herein over any one or more other transferase (or enzyme) involved in the ganglioside biosynthetic pathway (e.g., over all other transferases involved in the ganglioside biosynthetic pathway. In certain embodiments, ganglioside synthesis modulator (e.g., inhibitor or promoter) described herein is a selective ganglioside synthesis modulator (e.g., inhibitor or promoter) that modulates (e.g., inhibits) any specific transferase (or other enzyme) described herein as being involved in the ganglioside biosynthetic pathway over any one or more transferase (or other enzyme) involved in the biosynthetic pathway of a non-ganglioside glycan (e.g., N-linked glycan, glycosaminoglycan, O-linked glycan, or the like).

In certain embodiments, a selective ganglioside synthesis modulator (e.g., inhibitor or promoter) is selective for (i.e., directly or indirectly inhibits the activity of) a specific enzyme (e.g., transferase) in a ratio of greater than 1000:1 over one or more other enzyme (e.g., another enzyme involved in the ganglioside biosynthetic pathway, and/or another enzyme involved in the biosynthetic pathway of a non-ganglioside glycan). In specific embodiments, a selective ganglioside synthesis modulator (e.g., inhibitor or promoter) is selective for (i.e., directly or indirectly inhibits the activity of) a specific enzyme in a ratio of greater than 500:1 over one or more other enzyme (e.g., another enzyme involved in the ganglioside biosynthetic pathway, and/or another enzyme involved in the biosynthetic pathway of a non-ganglioside glycan). In specific embodiments, a selective ganglioside synthesis modulator (e.g., inhibitor or promoter) is selective for (i.e., directly or indirectly inhibits the activity of) a specific enzyme in a ratio of greater than 250:1, greater than 100:1, greater than 50:1, greater than 25:1, greater than 20:1, greater than 10:1, greater than 5:1, greater than 3:1, or greater than 2:1 over one or more other enzyme (e.g., another enzyme involved in the ganglioside biosynthetic pathway, and/or another enzyme involved in the biosynthetic pathway of a non-ganglioside glycan).

### Cellular Activity

In some embodiments, provided herein is a glycolipid modulator (e.g., a selective ganglioside synthesis inhibitor) having suitable cell availability and/or bioavailability to significantly effect the in cyto and/or in vivo biosynthesis of a glycolipid (e.g., a specific glycolipid in certain instances wherein a selective glycolipid synthesis modulator is utilized) when the glycolipid modulator is administered to a cell or individual, respectively. In certain instances, a significant effect is one wherein a measurable effect, a statistically significant effect, and/or a therapeutic effect is provided to the cell or individual. In certain specific embodiments, the specific glycolipid modulator is substantially cell permeable (e.g., when in contact with a cell, a significant percentage/amount of the modulator permeates the cell membrane). In some embodiments, the glycolipid modulator provides a statistically significant effect and/or therapeutic effect in a cell or individual at a non-toxic concentration, a substantially non-toxic concentration, a concentration below LC₅₀, a concentration below LC₂₀, a concentration below LC₀₁, or the like.

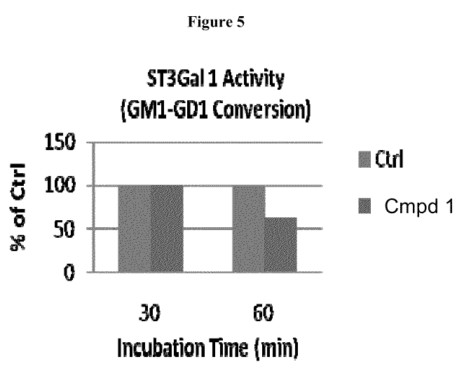

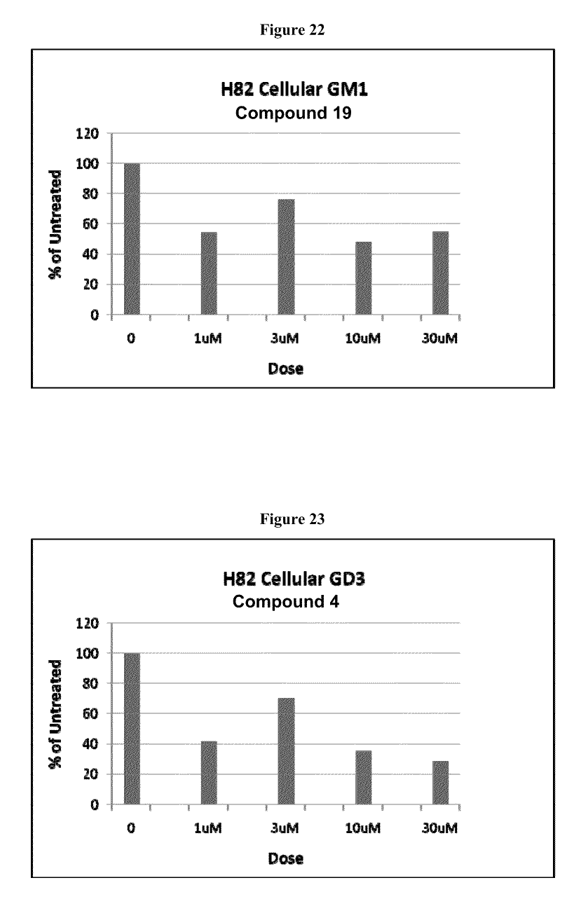

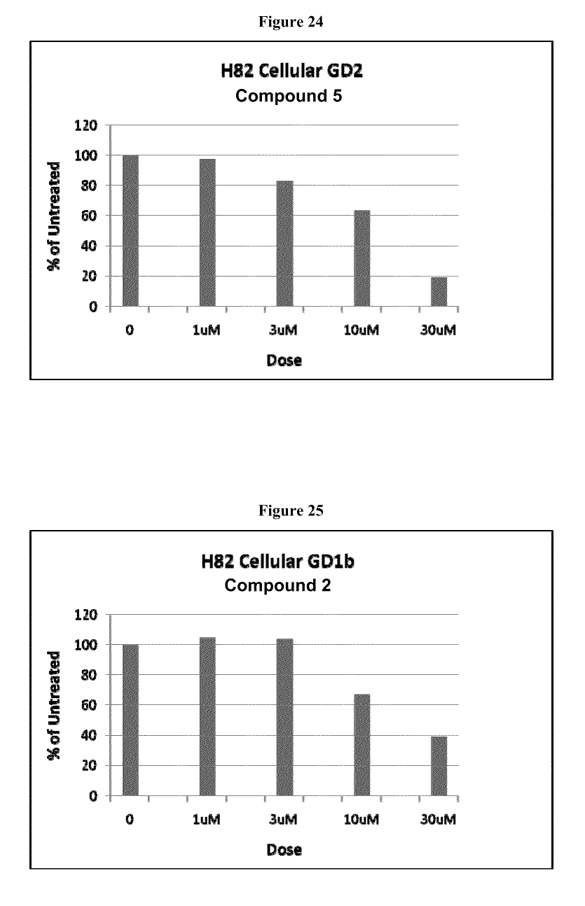

FIGS. 5-25 illustrate the specificity of affects of ganglioside synthesis modulator (e.g., inhibitor or promoter) compounds on the biosynthesis of ganglioside synthesis modulators (e.g., inhibitors or promoters).

In order for ganglioside synthesis modulators to have therapeutic benefit, modification (e.g., inhibition and/or promotion) of ganglioside biosynthesis must have cellular activity (e.g., the ganglioside synthesis modulators must be intracellularly active). Achieving cellular activity has generally been elusive in the field.

### Compounds

In certain embodiments, ganglioside synthesis inhibitors described herein are inhibitors of one or more of a GT_{1b} synthase including, a GM₃ synthase (e.g., sialyl transferase I), a GM₂/GD₂ synthase (e.g., β1-4 GalNAc transferase), a GD₃ synthase (e.g. sialyl transferase II), Gal TII, or Sialyl TIV, as compared to a GM₁ synthase, a GD₁ synthase, a GT_{1aα} synthase, or a GT_{1c} synthase. In more specific embodiments, ganglioside synthesis modulators selectively modulate (e.g., promote or inhibit) one or more of β-galactoside α-2,3-sialyltransferase (ST3), α-N-acetyl-neuraminide α-2,8-sialyltransferase 1 (ST8), (α-N-acetyl-neuraminyl-2,3-beta-galactosyl-1,3)-N-acetylgalactosaminide α-2,6-sialyltransferase 3 (ST6), (α-N-acetyl-neuraminyl-2,3-beta-galactosyl-1,3)-N-acetylgalactosaminide α-2,6-sialyltransferase 4 (ST6), (α-N-acetyl-neuraminyl-2,3-β-galactosyl-1,3)-N-acetylgalactosaminide α-2,6-sialyltransferase 5 (ST6), (α-N-acetyl-neuraminyl-2,3-β-galactosyl-1,3)-N-acetylgalactosaminide α-2,6-sialyltransferase 6 (ST6), β-1,4-N-acetyl-galactosaminyl transferase 1, or UDP-Gal:βGlcNAc β1,3-galactosyltransferase, polypeptide 4, or a combination thereof.

In certain embodiments, ganglioside synthesis modulator (e.g., inhibitor or promoter) described herein are small molecule organic compounds. Thus, in certain instances, ganglioside synthesis modulator (e.g., inhibitor or promoter) utilized herein are not polypeptides and/or carbohydrates. In some embodiments, in certain embodiments, a small molecule organic compound has a molecular weight of less than about 2,000 g/mol, less than about 1,500 g/mol, less than about 1,000 g/mol, less than 700 g/mol or less than about 500 g/mol. In specific embodiments, ganglioside synthesis modulators (e.g., inhibitors or promoters) described herein are non-carbohydrate compounds. In certain instances, carbohydrates tend to be hydrophilic due to the polyhydroxyls and therefore do not diffuse into cells efficiently. In some instances, carbohydrates have pharmacokinetic and pharmacodynamic properties in animals that are inappropriate for therapeutic drug effects. Further, carbohydrate hydroxyls may be reactive and make carbohydrates difficult and expensive to synthesize. The range of possible structures is limited compared to noncarbohydrate small molecules limiting the range of structural diversity. Moreover, in some instances, carbohydrates are not known to cross the blood-brain barrier. Noncarbohydrate small molecules are much less likely to be immunogenic or immunoreactive than are carbohydrates.

As used herein, carbohydrates include polyhydroxyaldehydes, polyhydroxyketones and their simple derivatives or larger compounds that can be hydrolyzed into such units. Carbohydrates also include polyhydroxyaldehydes, polyhydroxyketones and their simple derivatives that have been modified such that when they enter cells they are reconverted into polyhydroxyaldehydes, polyhydroxyketones. Carbohydrates also include sugar mimetics such as imino structures and alkaloids that inhibit glycosidases such as Deoxynojirimycin, Castanospermine, Australine, Deoxymannojirimycin, Kifunensen, Swainsonine and Mannostatin (page 709 of Essentials of Glycobiology second edition 2008 CSHL Press, CSH, New York.) In some instances, non carbohydrate small molecules are organic compounds containing less than 3 linked hydroxyl groups with a molecular weight of less than 700 Daltons.

In some instances, glycan inhibitors may be glycans (glycomimetics). In certain instances, a disadvantage to using glycomimetics is that it can be expensive, slow, and it involves complicated chemistry. In some instances, these disadvantages may severely limit the range of structures that can be tested. In certain instances, in order to have therapeutic efficacy, glycan biosynthetic inhibitors should enter cells and in further instances be able to enter subcellular organelles (endoplasmic reticulum and golgi) to gain access to the glycan biosynthetic enzymes. Due to the hydrophilic nature of carbohydrates, they are generally modified in order to provide a compound capable of entering these compartments. In some embodiments, provided herein are non-carbohydrate ganglioside biosynthesis inhibitors, which are cell penetrant and cell active. In certain embodiments, use of non-carbohydrate biosynthesis inhibitors allows for compounds that avoid the disadvantages associated with carbohydrate glycan inhibitors.

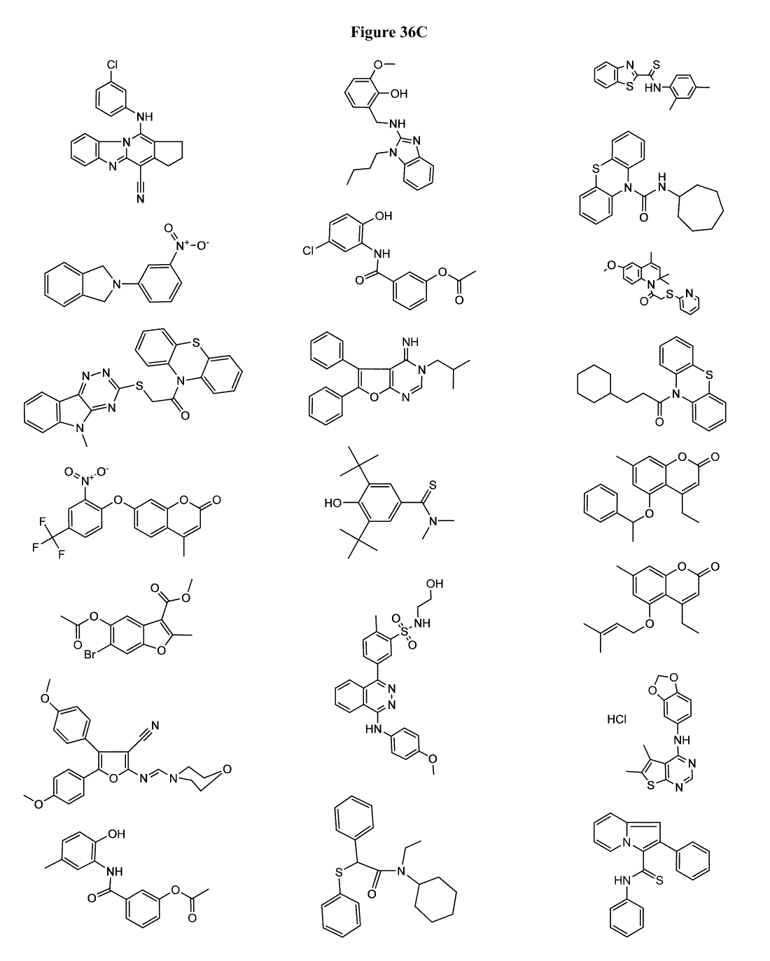

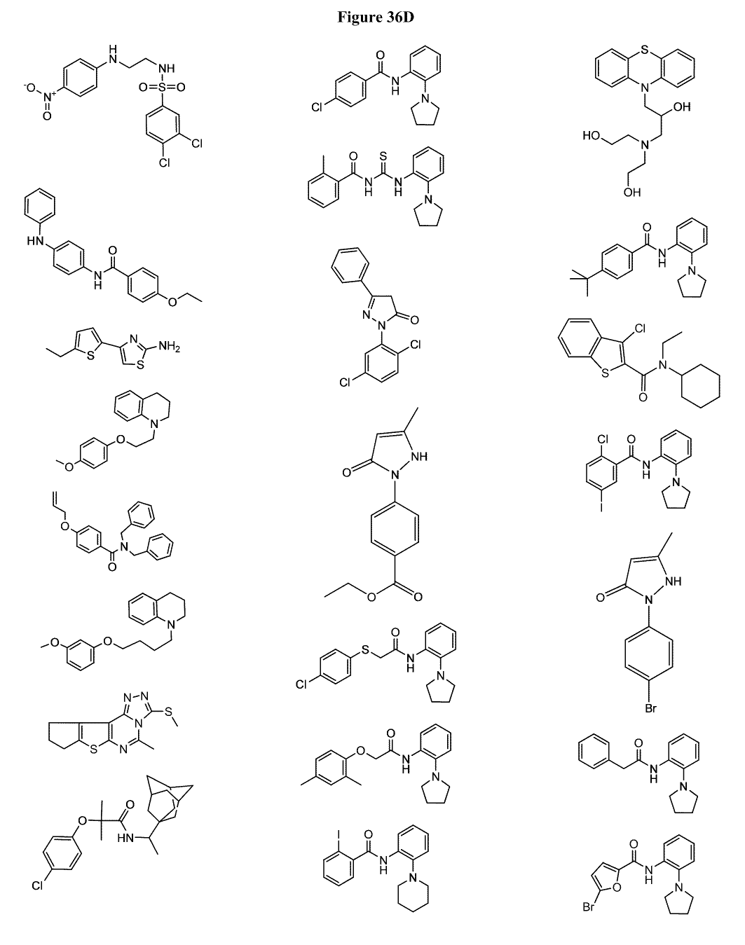

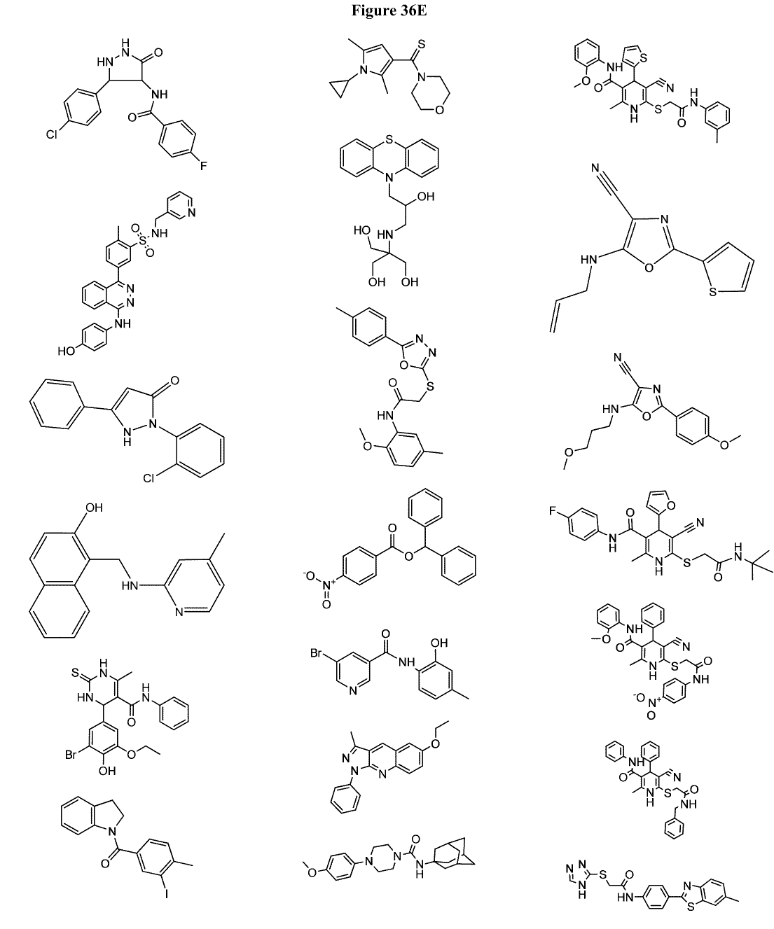

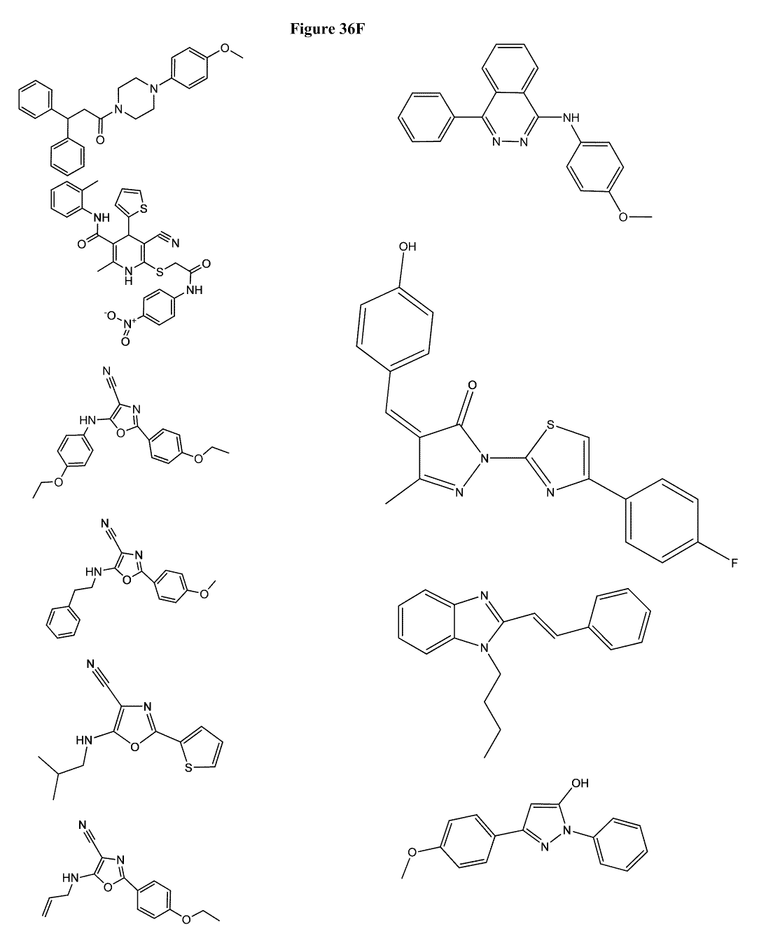

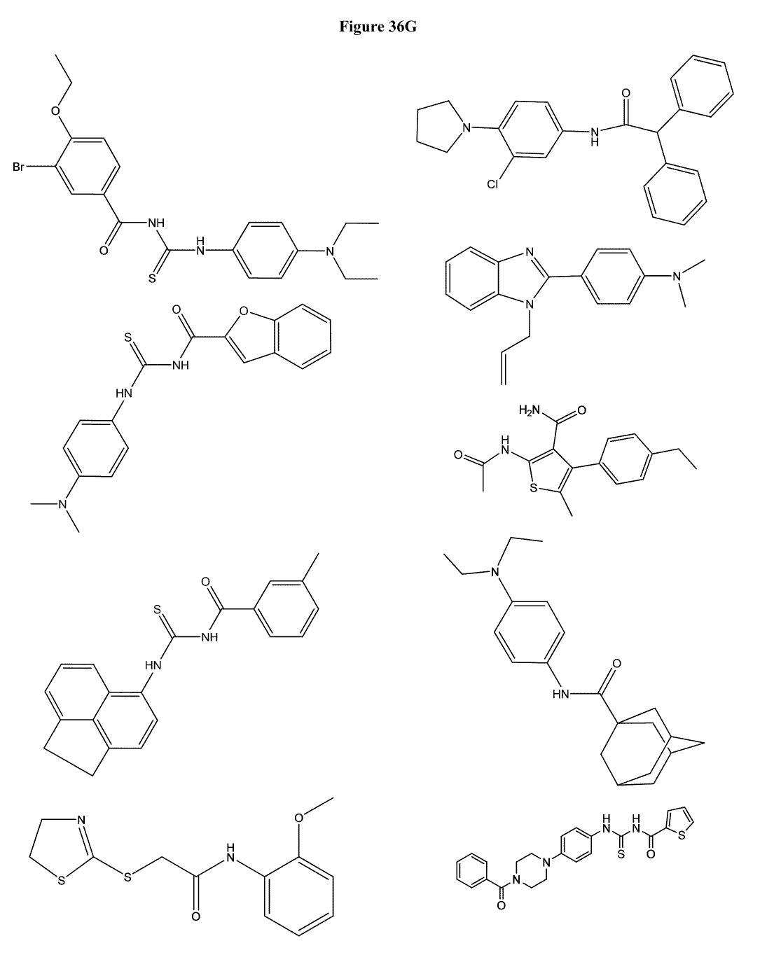

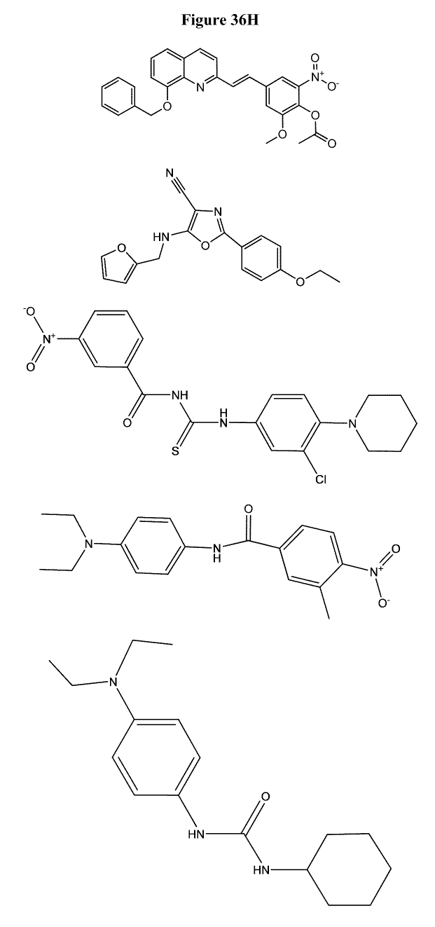

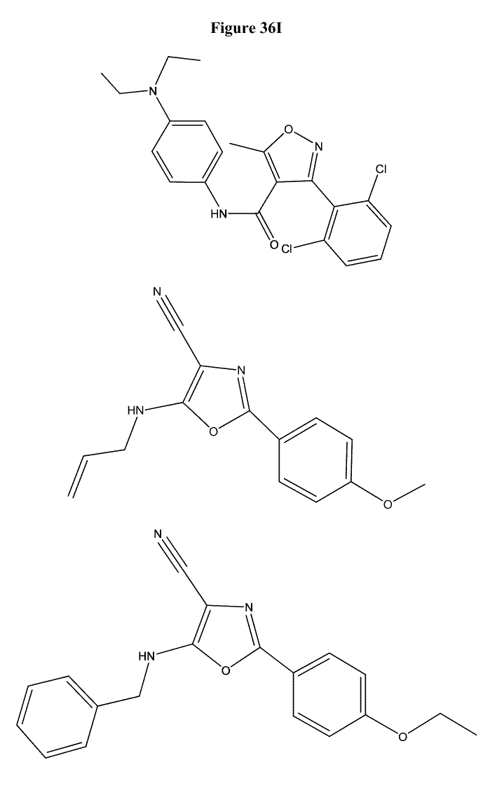

In some embodiments, selective modulators (e.g., inhibitors or promoters) of ganglioside biosynthesis include compounds of any of FIGS. 36A-36I. In certain embodiments, selective modulators (e.g. inhibitors or promoters) of ganglioside biosynthesis include, but are not limited to, the following compounds: 4-(2-chlorobenzyl)-N-((3,5-dimethyl-1-phenyl-1H-pyrazol-4-yl)methylene)piperazin-1-amine (1); 4,6-di-tert-butyl-2-(4-(dimethylamino)phenyl)benzo[d]oxazol-7-ol (2); 2-(5-(4-(methylthio)phenyl)-1H-1,2,4-triazol-1-yl)-5,6,7,8-tetrahydro-4H-cyclohepta[b]thiophene-3-carbonitrile (3); 2-(5-(4-fluorophenyl)-1H-1,2,4-triazol-1-yl)-5,6,7,8-tetrahydro-4H-cyclohepta[b]thiophene-3-carbonitrile (4); 2-(5-bromo-2-hydroxyphenyl)-4,6-di-tert-butylbenzo[d]oxazol-7-ol (5); 4-methyl-7-(2-nitro-4-(trifluoromethyl)phenoxy)-2H-chromen-2-one (6); 6-(2-(benzylamino)-2-oxoethylthio)-5-cyano-2-methyl-N,4-diphenyl-1,4-dihydropyridine-3-carboxamide (7); 5-(isobutylamino)-2-(thiophen-2-yl)oxazole-4-carbonitrile (8); 3-(4-(pyridin-4-yl)thiazol-2-ylamino)phenol (10); 4-hydroxy-N-(4-hydroxyphenyl)-2-oxo-1,2-dihydroquinoline-3-carboxamide (11); 2-phenoxyethyl 4-(3-chloro-5-ethoxy-4-hydroxyphenyl)-7-(4-chlorophenyl)-2-methyl-5-oxo-1,4,5,6,7,8-hexahydroquinoline-3-carboxylate (12); (Z)-1-(4-(4-fluorophenyl)thiazol-2-yl)-4-(4-hydroxybenzylidene)-3-methyl-1H-pyrazol-5(4H)-one (13); 1-(4-(4-chlorophenyl)thiazol-2-yl)-3,4-dimethyl-1H-pyrazol-5(4H)-one (14); (E)-4-(2-(8-(benzyloxy)quinolin-2-yl)vinyl)-2-methoxy-6-nitrophenyl acetate (15); N-(1-adamantyl)-4-(4-methoxyphenyl)piperazine-1-carboxamide (16); 4-(4-chlorophenyl)-N-((5-methylfuran-2-yl)methylene)piperazin-1-amine (17); 4-(4-bromophenylimino)-2,6-di-tert-butylcyclohexa-2,5-dienone (18); (E)-2-(2-(1-(3,4-dimethoxyphenyl)ethylidene)hydrazinyl)-3-methylquinoxaline (19); 4,6-di-tert-butyl-2-(2,3-dimethoxyphenyl)benzo[d]oxazol-7-ol (20); 2-(4-chloro-3,5-dimethylphenoxy)-N-(4-(dimethylamino)phenyl)acetamide (21). In some embodiments, other ganglioside biosynthesis inhibitors, including selective biosynthesis inhibitors, include other compounds identified according to any process described herein.

Selective ganglioside synthesis modulators (e.g., inhibitor or promoter) inhibit binding of CTB to cellular glycans, but not PHA (N-linked), FGF (HS), WGA (Sialic acid and terminal GlcNAc). Modulators (e.g., inhibitors) of glycan synthesis include agents that act directly on the relevant biosynthetic enzymes or indirectly on other targets (e.g. protein kinase, phosphatase, transporter, GPCR, ion channel, hormone receptor, protease, etc.) that would alter the structure of the glycans though effects on biosynthetic (anabolic) enzymes or degradative (catabolic) enzymes.

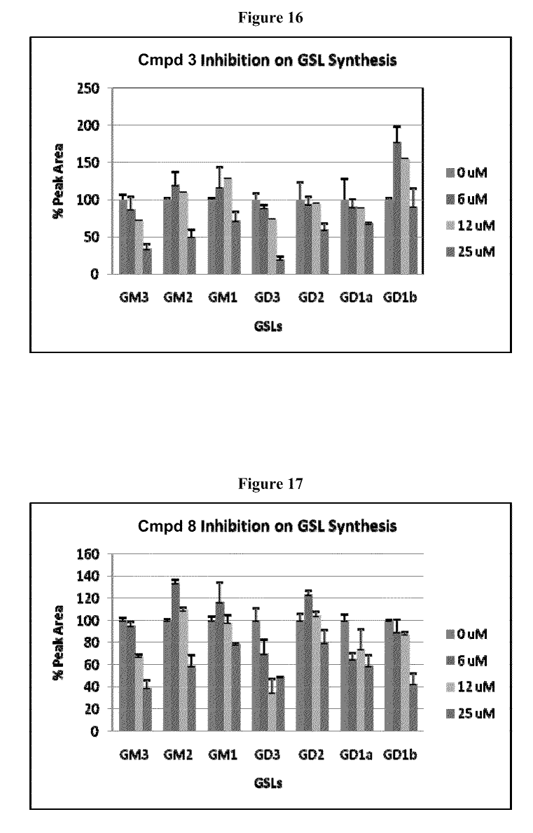

Selective inhibitors are identified using any suitable process, such as described herein. For example, in some embodiments, specific modifiers preferentially inhibit synthesis of GM₃ and GD₃ relative to the other ganglioside species. FIGS. 16 and 17 illustrate a process described herein whereby preferential inhibition of GM₃ and GD₃ relative to other ganglioside species is identified. In some instances, ganglioside biosynthesis inhibitors that specifically target GM₃ and GD₃ provide a reduction in other gangliosides. For example, based on the biosynthetic pathway (see FIG. 4), the data in FIGS. 16 and 17 suggest that compounds that specifically target GM₃ and GD₃ provide for the reduction in other gangliosides as a result of the reduction in GM₃ and GD₃.

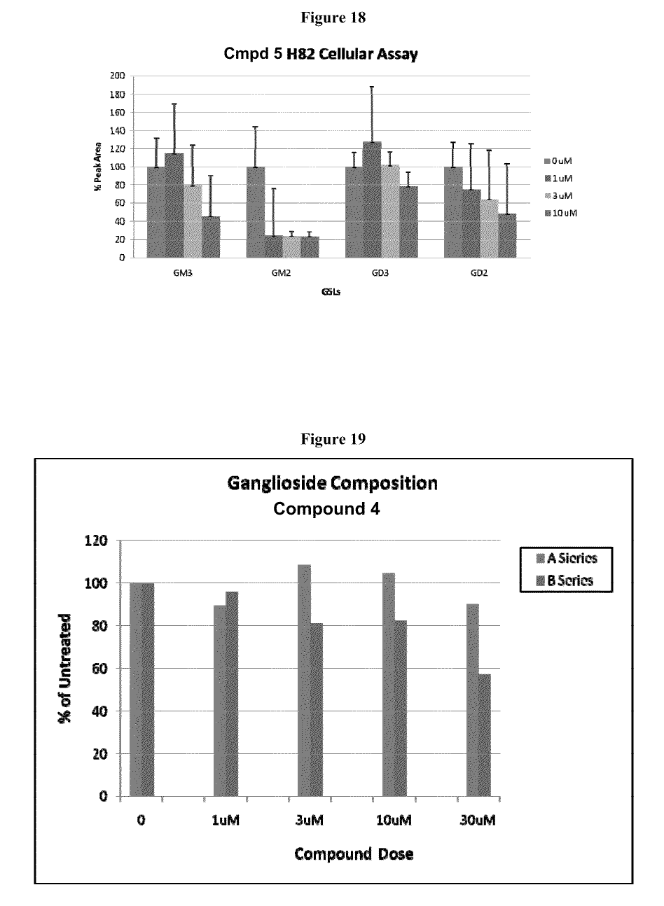

In certain embodiments, specific or selective modifiers preferentially inhibit the biosynthesis of one series of gangliosides relative to another series of gangliosides (see FIG. 4, horizontal numerical series). FIG. 18 illustrates the results of a process described herein whereby specific reduction of one series of gangliosides relative to another series of gangliosides is identified. For example, the data in FIG. 18 suggest that a specific modifier of ganglioside biosynthesis provided for a preferential reduction of 2 series gangliosides (GM₂, GD₂) relative to 3 series gangliosides (GD₃, GM₃).

In some embodiments, specific or selective modifiers reduce a series of gangliosides relative to another series of gangliosides (see FIG. 4, vertical alphabetical series). FIG. 19 illustrates the results of a process described herein whereby specific reduction of one series of gangliosides relative to another series of gangliosides is identified. For example, the data in FIG. 19 suggest that a specific modifier of ganglioside biosynthesis provided for a reduction of the B series gangliosides (GD₃, GD₂, GD_{1b}, GT_{1b}, GQ_{1b}) relative to A series gangliosides (GM₃, GM₂, GM₁, GD₁ₐ).

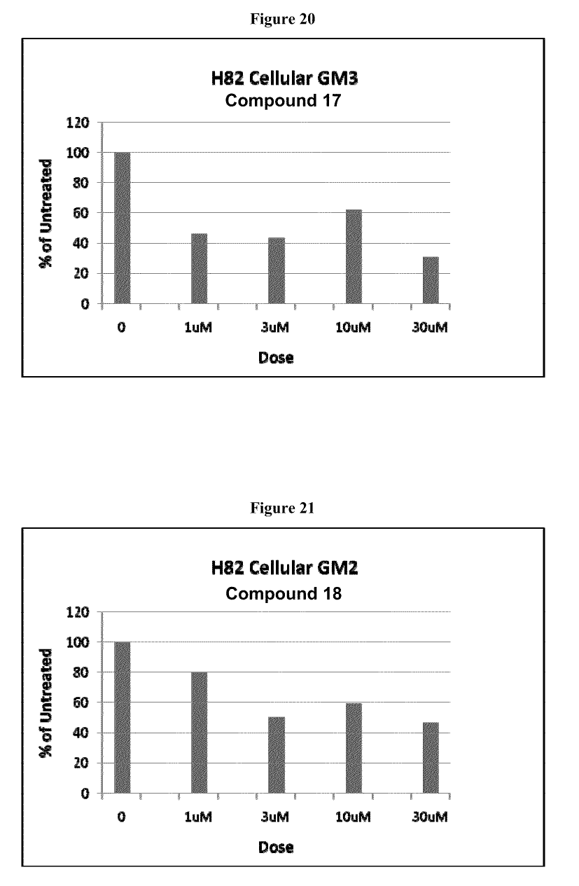

In certain embodiments, inhibitors directed at blocking the biosynthesis of only the ganglioside subset of GSLs have dose dependent reduction (inhibition) effects on individual gangliosides (individual ganglioside HPLC peaks). FIGS. 20-25 illustrate a process described herein whereby specific reduction of individual gangliosides is identified. For example, the data in FIGS. 20-25 suggest that specific inhibitors of ganglioside biosynthesis provide for a reduction in the individual gangliosides selected from, but not limited to, GM₃, GM₂, GM₁, GD₃, GD₂, GD_{1b}.

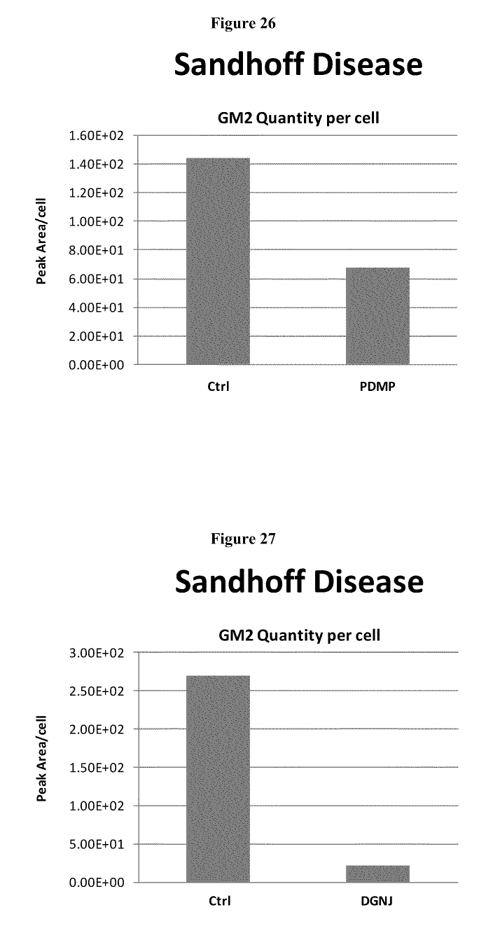

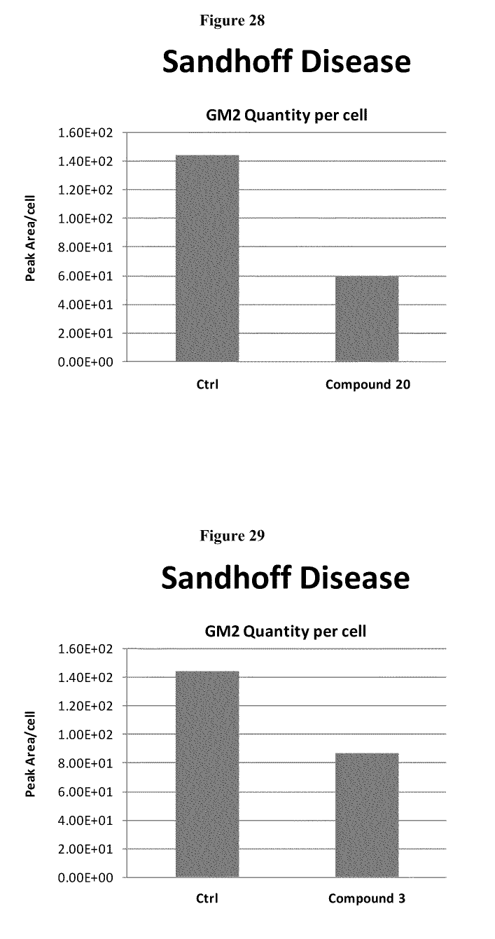

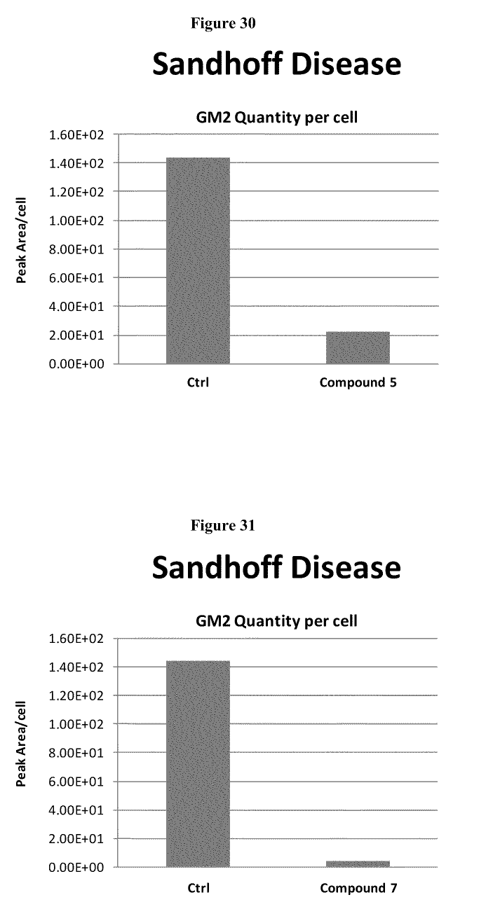

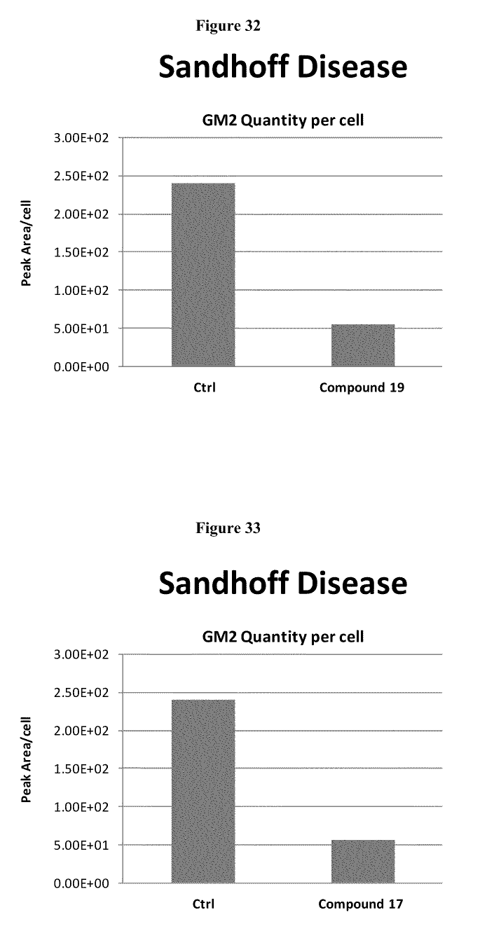

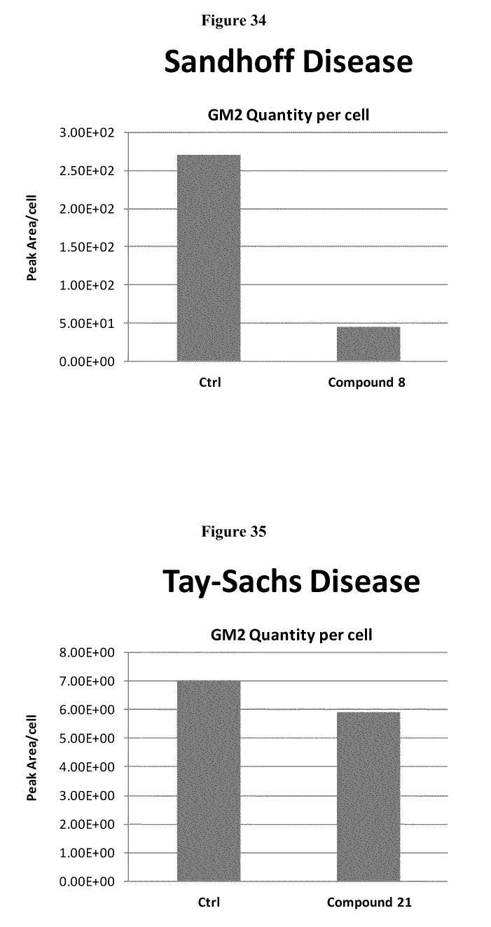

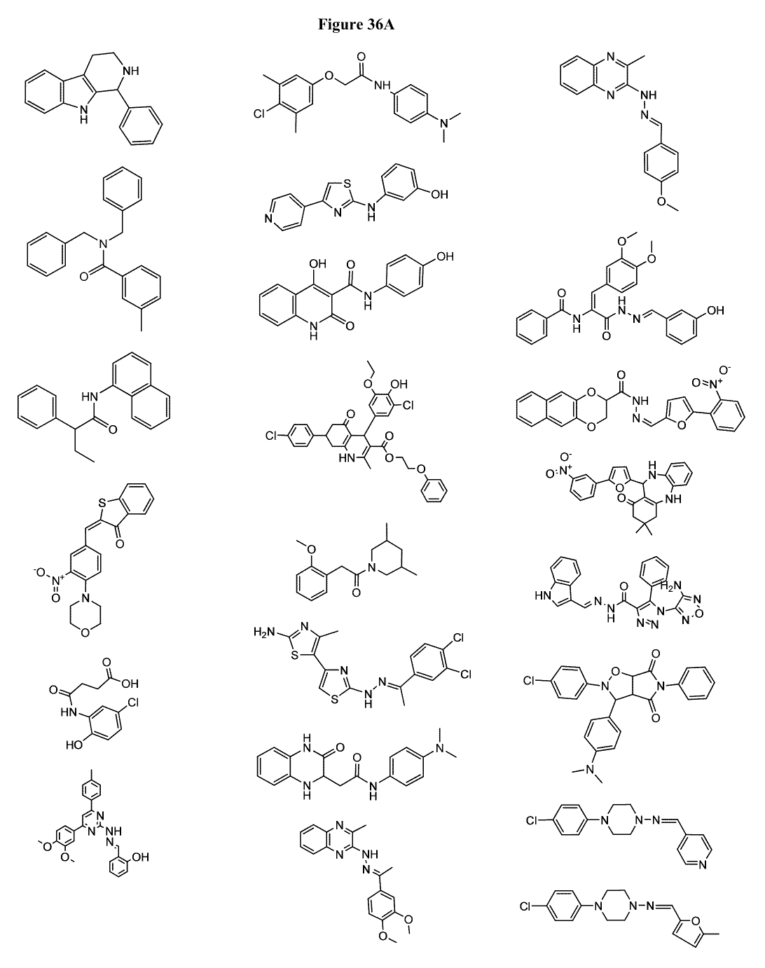

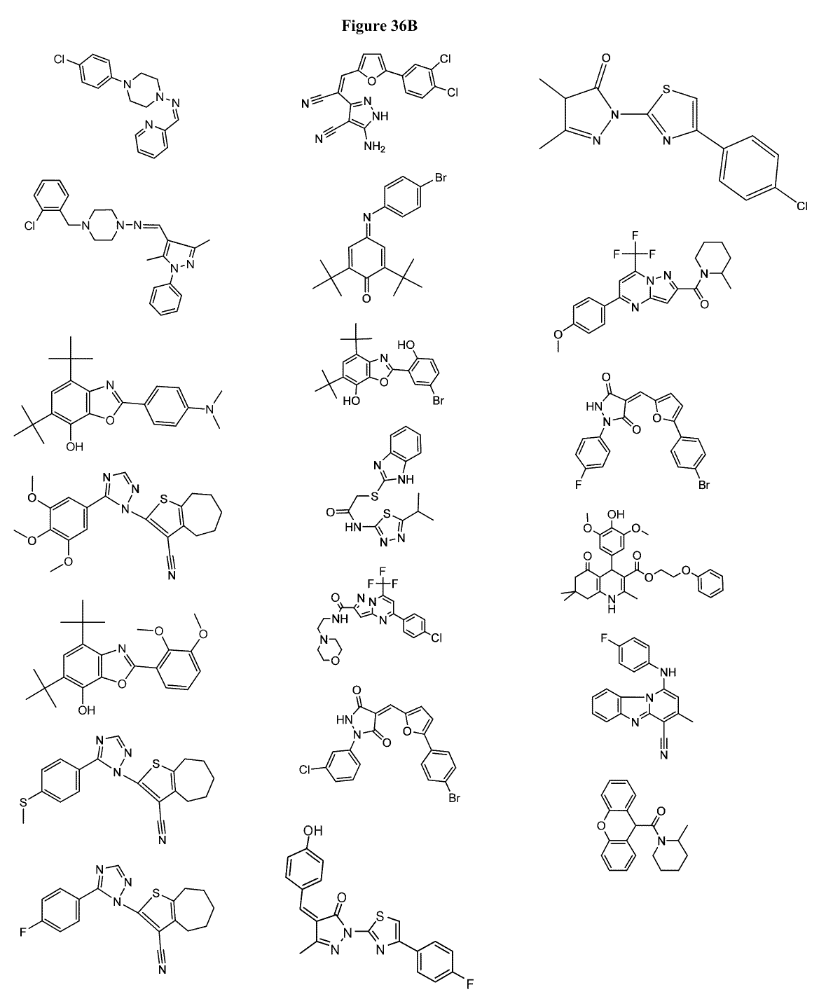

In some embodiments, glycolipid inhibitors reduce GM₂ storage in primary human fibroblasts. FIGS. 26-35 illustrate a process described herein whereby the reduction of GM₂ storage in primary human fibroblasts is identified. For example, FIGS. 26 and 27 illustrate the activity of the known non-selective glycolipid inhibitors PDMP and DGNJ, respectively. In certain embodiments, a more specific inhibitor directed at blocking the biosynthesis of only the ganglioside subset of GSLs reduces unwanted side effects due to the inhibition of all GSLs. Inhibitors of for example GM3 synthase (ST3Gal-V), GM2/GD2 synthase (β1-4 GalNAc transferase), GD3 synthase (ST8Sial-I), Gal TII, ST3Gal-II or other downstream enzymes would affect only the ganglioside family. FIGS. 28-35 illustrate a process described herein whereby the reduction of GM2 storage in primary human fibroblasts by selective inhibitors of ganglioside biosynthesis is identified. For example, the data in FIGS. 28-34 suggest that specific inhibitors of ganglioside biosynthesis provide for a reduction of GM₂ storage in primary human fibroblasts from Sandhoff disease patients. And for instance, the data in FIG. 35 suggest that specific inhibitors of ganglioside biosynthesis provide for a reduction of GM₂ storage in primary human fibroblasts from Tay-Sachs disease patients.

### GENERAL DEFINITIONS

The term “subject”, “patient” or “individual” are used interchangeably herein and refer to mammals and non-mammals, e.g., suffering from a disorder described herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” and other grammatical equivalents as used herein, include alleviating, inhibiting or reducing symptoms, reducing or inhibiting severity of, reducing incidence of, prophylactic treatment of, reducing or inhibiting recurrence of, delaying onset of, delaying recurrence of, abating or ameliorating a disease or condition symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the patient.

The terms “prevent,” “preventing” or “prevention,” and other grammatical equivalents as used herein, include preventing additional symptoms, preventing the underlying metabolic causes of symptoms, inhibiting the disease or condition, e.g., arresting the development of the disease or condition and are intended to include prophylaxis. The terms further include achieving a prophylactic benefit. For prophylactic benefit, the compositions are optionally administered to a patient at risk of developing a particular disease, to a patient reporting one or more of the physiological symptoms of a disease, or to a patient at risk of reoccurrence of the disease.

Where combination treatments or prevention methods are contemplated, it is not intended that the agents described herein be limited by the particular nature of the combination. For example, the agents described herein are optionally administered in combination as simple mixtures as well as chemical hybrids. An example of the latter is where the agent is covalently linked to a targeting carrier or to an active pharmaceutical. Covalent binding can be accomplished in many ways, such as, though not limited to, the use of a commercially available cross-linking agent. Furthermore, combination treatments are optionally administered separately or concomitantly.

As used herein, the terms “pharmaceutical combination”, “administering an additional therapy”, “administering an additional therapeutic agent” and the like refer to a pharmaceutical therapy resulting from the mixing or combining of more than one active ingredient and includes both fixed and non-fixed combinations of the active ingredients. The term “fixed combination” means that at least one of the agents described herein, and at least one co-agent, are both administered to a patient simultaneously in the form of a single entity or dosage. The term “non-fixed combination” means that at least one of the agents described herein, and at least one co-agent, are administered to a patient as separate entities either simultaneously, concurrently or sequentially with variable intervening time limits, wherein such administration provides effective levels of the two or more agents in the body of the patient. In some instances, the co-agent is administered once or for a period of time, after which the agent is administered once or over a period of time. In other instances, the co-agent is administered for a period of time, after which, a therapy involving the administration of both the co-agent and the agent are administered. In still other embodiments, the agent is administered once or over a period of time, after which, the co-agent is administered once or over a period of time. These also apply to cocktail therapies, e.g. the administration of three or more active ingredients.

As used herein, the terms “co-administration”, “administered in combination with” and their grammatical equivalents are meant to encompass administration of the selected therapeutic agents to a single patient, and are intended to include treatment regimens in which the agents are administered by the same or different route of administration or at the same or different times. In some embodiments the agents described herein will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, the agents described herein and the other agent(s) are administered in a single composition. In some embodiments, the agents described herein and the other agent(s) are admixed in the composition.

The terms “effective amount” or “therapeutically effective amount” as used herein, refer to a sufficient amount of at least one agent being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In specific instances, the result is the alteration of or the disruption of the structure of endogenous ganglioside such that the binding ability, signaling ability or combination thereof of the ganglioside is inhibited or reduced. In certain instances, an “effective amount” for therapeutic uses is the amount of the composition comprising an agent as set forth herein required to provide a clinically significant decrease in a disease. An appropriate “effective” amount in any individual case is determined using any suitable technique, such as a dose escalation study.

The terms “administer,” “administering”, “administration,” and the like, as used herein, refer to the methods that may be used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Administration techniques that can be employed with the agents and methods described herein include, e.g., as discussed in Goodman and Gilman, The Pharmacological Basis of Therapeutics, current ed.; Pergamon; and Remington's, Pharmaceutical Sciences (current edition), Mack Publishing Co., Easton, Pa. In certain embodiments, the agents and compositions described herein are administered orally.

The term “nascent ganglioside” as used herein refers to any glycolipid (e.g., a ceramide-linked glycan) that is subject to further modification (e.g., polymerization, sialylation). In some embodiments, a nascent ganglioside is e.g., a LacCer moiety, a G_{M3} ganglioside, a G_{D3} ganglioside, a G_{D2} ganglioside, a G_{D1b} ganglioside or the like.

The term “O series of gangliosides” or “O-gangliosides” refers to G_{A2}, G_{A1}, G_{M1b}, G_{D1c} gangliosides or the like and is used interchangeably with the term(s) G_{A2} ganglioside, G_{A1} ganglioside, G_{M1b} ganglioside, G_{D1c} ganglioside, or the like. The term “A series of gangliosides” or “A ganglioside” refers to G_{M3}, G_{M2}, G_{M1a}, G_{D1a}, G_{T1a} gangliosides or the like and is used interchangeably with the terms(s) G_{M3} ganglioside, G_{M2} ganglioside, G_{M1a} ganglioside, G_{D1a} ganglioside, G_{T1a} ganglioside or the like. The term “B series of gangliosides” or “B ganglioside” refers to G_{D3}, G_{D2}, G_{D1b}, G_{T1b}, G_{D1b} gangliosides or the like and is used interchangeably with the terms(s) G_{D3} ganglioside, G_{D2} ganglioside, G_{D1b} ganglioside, G_{T1a} ganglioside, G_{D1b} ganglioside or the like. The term “C series of gangliosides” or “C ganglioside” refers to G_{T3}, G_{T2}, G_{T1c}, G_{Q1c}, G_{P1c} gangliosides or the like and is used interchangeably with the terms(s) G_{T3} ganglioside, G_{T2} ganglioside, G_{T1c} ganglioside, G_{Q1c} ganglioside, G_{P1c} ganglioside or the like.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material may be administered to an individual without causing significant undesirable biological effects or significantly interacting in a deleterious manner with any of the components of the composition in which it is contained.

The term “carrier” as used herein, refers to relatively nontoxic chemical agents that, in certain instances, facilitate the incorporation of an agent into cells or tissues.

The symbolic nomenclature used herein follows the “Symbol and Text Nomenclature for Representation of Glycan Structure” as promulgated by the Nomenclature Committee for the Consortium for Functional Glycomics, as amended on October 2007.

Ganglioside definition: Gangliosides are a subset of glycosphingolipid molecules composed of ceramide linked by a glycosidic bond to an oligosaccharide chain containing hexose and N-acetylneuraminic acid (NANA, acidic sugar known also as sialic acid) units.

In certain instances, the “ganglio” core contain four saccharide residues (or up to four residues) is designated by the letter G, followed by a letter designating the total number of sialic acid residues (M, mono; D, di; T, tri; Q, penta; A, asialo, none). The following number represents the length of the ganglio core, with 1 representing the full four-saccharide core, and shorter structures having higher numbers. Lower case letters designate the number of sialic acid residues linked to the internal Gal residue (a=1, b=2) and Greek letters indicate the number of sialic acids linked to the GalNAc residue (α=1, β=2).

As set forth by the Svennerholm nomenclature and outlined in FIG. 4, certain gangliosides are referred to as belonging to particular lettered series: O, A, B, and C. The O series or asialo refers to gangliosides containing no sialic acid residues bound the galactose residue in the ceramide linked disaccharide (1^{st} 2 residues of the ganglioside core). The A series or monosialylated refers to gangliosides containing one sialic acid residue bound (α 2,3) to the galactose residue in the ceramide linked disaccharide (1^{st} 2 residues of the ganglioside core). The B series or disialylated refers to gangliosides containing a sialic acid disaccharide bound (α 2,3) to the galactose residue in the ceramide linked disaccharide (1^{st} 2 residues of the ganglioside core). The C series or trisialylated refers to gangliosides containing a sialic acid trisaccharide bound (α 2,3) to the galactose residue in the ceramide linked disaccharide (1^{st} 2 residues of the ganglioside core).

As set forth by the Svennerholm nomenclature and outlined in FIG. 4, certain gangliosides are referred to as belonging to particular numbered series: 1, 2, 3 representing the length of the ganglioside core. The 3 series refers to gangliosides with disaccharide cores linked to ceramide of the sequence Gal-Glc-Cer. The 2 series refers to gangliosides with trisaccharide cores linked to the ceramide of the sequence GalNAc-Gal-Glc-Cer. The 1 series refers to gangliosides with the full tetrasaccharide core linked to ceramide of the sequence Gal-GalNAc-Gal-Glc-Cer.

As used herein, the names and short forms of enzymes are meant to encompass any alternative names commonly used for these enzymes. For example:

GM3-synthase is used interchangeably herein with ST3GalV transferase or ST-I,

GM2/GD2-synthetase is used interchangeably herein with β1-4 GalNAc transferase or GalNAc-T,

GM1/GD1b-synthase is used interchangeably herein with β1-3 Gal-II transferase or GalT-II,

GD1a/GT1b-synthase is used interchangeably herein with ST3Gal-I/II transferase or ST-IV, and

GD3-synthase is used interchangeably herein with ST8Sial-1 transferase or ST-II.

### Methods

Provided herein is a process for modifying the structure of a glycolipid (e.g., a ceramide-linked glycan), comprising contacting a cell that produces at least one ceramide-linked glycan with an effective amount of any glycolipid synthesis inhibitor described herein. In some embodiments, the glycolipid synthesis inhibitor is a ganglioside synthesis inhibitor. In some embodiments, the ganglioside synthesis inhibitor is a selective ganglioside synthesis inhibitor (e.g., inhibitor of a GT_{1b} ganglioside as compared to the inhibition of the function of other ceramide-linked glycans), e.g., as described herein. In some embodiments, the selective ganglioside synthesis inhibitor is a modulator of (e.g., promotes one or more of, or inhibits one or more of) a ceramide synthase (e.g., modulates LacCer synthase), ganglioside glycosylation (e.g., modulates a ganglioside glycosyltransferase), ganglioside sulfation (e.g., modulates a ganglioside sulfotransferase), ganglioside phosphorylation (e.g., modulates a ganglioside kinase), ganglioside degradation (e.g. modulates a glycosidase, a ceramidase) or a combination thereof.

In some embodiments, the ganglioside synthesis inhibitor modulates (e.g., promote or inhibit) a glycosyltransferase. In some embodiments, the inhibitor of a ganglioside glycosyltransferase inhibits the synthesis of the linkage region (e.g. via linkage of ceramide to a lactosyl moiety), the initiation of ganglioside synthesis (e.g. via inhibition of 1-O-acylceramide synthase), the synthesis of ganglioside (e.g., further sialylation of a ceramide-linked glycan), or a combination thereof. In some embodiments, ganglioside biosynthesis inhibitors selectively modulate one or more of a glycosyltransferase, a glucosyl transferase or a sialyl transferase. In some embodiments, ganglioside biosynthesis inhibitors selectively modulate (e.g., promote or inhibit) synthesis of one or more of O, A, B or C gangliosides. In specific embodiments, ganglioside synthesis inhibitors modulate one or more of a GT_{1b} synthase including, a GM₃ synthase (e.g., sialyl transferase I), a GM₂/GD₂ synthase (e.g., β1-4 GalNAc transferase), a GD₃ synthase (e.g. sialyl transferase II), Gal TII, or Sialyl TIV, as compared to a GM₁ synthase, a GD₁ synthase, a GT_{1aα} synthase, or a GT_{1c} synthase. In more specific embodiments, ganglioside synthesis modulators selectively modulate (e.g., promote or inhibit) one or more of β-galactoside α-2,3-sialyltransferase (ST3), α-N-acetyl-neuraminide α-2,8-sialyltransferase 1 (ST8), (α-N-acetyl-neuraminyl-2,3-beta-galactosyl-1,3)-N-acetylgalactosaminide α-2,6-sialyltransferase 3 (ST6), (α-N-acetyl-neuraminyl-2,3-beta-galactosyl-1,3)-N-acetylgalactosaminide α-2,6-sialyltransferase 4 (ST6), (α-N-acetyl-neuraminyl-2,3-β-galactosyl-1,3)-N-acetylgalactosaminide α-2,6-sialyltransferase 5 (ST6), (α-N-acetyl-neuraminyl-2,3-β-galactosyl-1,3)-N-acetylgalactosaminide α-2,6-sialyltransferase 6 (ST6), β-1,4-N-acetyl-galactosaminyl transferase 1, or UDP-Gal:βGlcNAc β1,3-galactosyltransferase, polypeptide 4, or a combination thereof.

In certain embodiments, the effective amount of the ganglioside synthesis inhibitor alters or disrupts the nature (e.g., alters or disrupts the sialylation, glycosylation, concentration of gangliosideschain length of ganglioside, or a combination thereof) of ganglioside compared to endogenous ganglioside in an amount sufficient to alter or disrupt ganglioside binding, ganglioside signaling, or a combination thereof. In specific embodiments, the ganglioside synthesis inhibitor described herein alters or disrupts the nature of the ganglioside (e.g., nature of O, A, B or C gangliosides) such that it inhibits ganglioside signaling. In other specific embodiments, the ganglioside synthesis inhibitor described herein alters or disrupts the nature of the ganglioside such that it inhibits ganglioside binding. In specific embodiments, the ganglioside synthesis inhibitor described herein alters or disrupts the nature of the ganglioside (e.g., nature of O, A, B or C gangliosides) such that it inhibits ganglioside binding. In more specific embodiments, the ganglioside synthesis inhibitor described herein alters or disrupts the nature of the ganglioside such that it inhibits ganglioside binding and ganglioside signaling. In specific embodiments, the ganglioside synthesis inhibitor described herein alters or disrupts the nature of the ganglioside (e.g., nature of O, A, B or C gangliosides) such that it inhibits ganglioside signaling and binding. In some embodiments, the ganglioside synthesis inhibitor alters or disrupts the nature of the ganglioside such that it inhibits the binding, signaling, or a combination thereof of any lectin (including polypeptides) subject to ganglioside binding, signaling or a combination thereof, in the absence of a ganglioside synthesis inhibitor. In some embodiments, the polypeptide is, by way of non-limiting example, a cell adhesion molecule (CAM). In certain embodiments, the CAM is an exogenous CAM, e.g., a bacterial lectin. In certain embodiments, the CAM is an endogenous CAM and includes, by way of non-limiting examples, E-selectin, L-selectin or P-selectin.

In certain embodiments, the cell is present in an individual (e.g., a human) diagnosed with a disorder or condition mediated by ganglioside. In certain instances, the disorder mediated by ganglioside is a cancer, a tumor, undesired angiogenesis (e.g., cancer, diabetic blindness, age-related macular degeneration, rheumatoid arthritis, or psoriasis), insufficient angiogenesis (e.g., coronary artery disease, stroke, or delayed wound healing), mucopolysaccharidosis, amyloidosis, a spinal cord injury, hypertriglyceridemia, inflammation, a wound, or the like. In some embodiments, the cell is present in a human diagnosed with cancer. In certain embodiments, the cell is present in an individual (e.g., a human) diagnosed with abnormal angiogenesis and/or undesired angiogenesis. In some embodiments, the cell is present in an individual (e.g., a human) diagnosed with a lysosomal storage disease (e.g., mucopolysaccharidosis (MPS)). In some embodiments, the individual is diagnosed with MPS I, MPS II, or MPS III. In some embodiments, the cell is present in an individual (e.g., a human) diagnosed with amyloidosis, a spinal cord injury, hypertriglyceridemia, inflammation, or the like. In some embodiments, the cell is present in an individual diagnosed with Salidosis, Tay Sachs, Sandhoff, GM₁ gangliosidosis, or Fabry disease.

In some embodiments, the cell is present in an individual (e.g., a human) diagnosed with sialidosis, sialuria, thrombocytopenia, leukopenia, tumorous calcinosis, Alzheimer's disease, Parkinson's disease, Huntington's disease, spongiform encephalopathies (Creutzfeldt-Jakob, Kuru, Mad Cow), diabetic amyloidosis, type-2 diabetes, Rheumatoid arthritis, juvenile chronic arthritis, Ankylosing spondylitis, psoriasis, psoriatic arthritis, adult still disease, Becet syndrome, familial Mediterranean fever, Crohn's disease, leprosy, osteomyelitis, tuberculosis, chronic bronchiectasis, Castleman disease, Hodgkin's disease, renal cell carcinoma, or carcinoma of the gut, lung or urogenital tract.

In some embodiments, the cell is present in an individual (e.g., human) diagnosed with pancreatic cancer, myeloma, ovarian cancer, hepatocellular cancer, breast cancer, colon carcinoma, brain cancer, brain tumor, neuroblastoma, or melanoma. In some embodiments, the cell is present in an individual (e.g., human) diagnosed with small cell lung cancer, large cell lung cancer, non-small cell lung cancer, or the like. In certain embodiments, the cell is a pancreatic cancer cell, myeloma cell, ovarian cancer cell, hepatocellular cancer cell, breast cancer cell, colon carcinoma cell, renal cell carcinoma, carcinoma of the gut, lung or urogenital tract, or melanoma cell. In some embodiments, the cell a small cell lung cancer cell, large cell lung cancer cell, non-small cell lung cancer cell, or the like.

In some embodiments, the cell is present in an individual (e.g., human) diagnosed with an infectious or viral disease including, by way of non-limiting example, herpes, diphtheria, papilloma virus, hepatitis, HIV, coronavirus, or adenovirus.

In certain embodiments, ganglioside synthesis inhibitors described herein are small molecule organic compounds. In certain instances, ganglioside synthesis inhibitors utilized herein are not polypeptides or carbohydrates. In some embodiments, a small molecule organic compounds has a molecular weight of less than about 2,000 g/mol, less than about 1,500 g/mol, less than about 1,000 g/mol, less than about 700 g/mol, or less than about 500 g/mol.

In other embodiments, provided herein are methods for treating sialyl transferase deficiency, the method comprising administering to a patient suffering from a disease or condition mediated by sialyl transferase deficiency a therapeutically effective amount of a ganglioside biosynthesis inhibitor. In one embodiment, the disease or condition mediated by sialyl transferase deficiency is thrombocytopenia, leukopenia, sialidosis, metachromatic leukodystrophy and sialuria. In one embodiment, the ganglioside biosynthesis inhibitor is an inhibitor of an α2,3-sialyl transferase, an α2,8-sialyl transferase or combination thereof.

In another embodiment, provided herein is a method for treating GalNAc transferase deficiency, the method comprising administering to a patient suffering from a disease or condition mediated by GalNAc transferase deficiency a therapeutically effective amount of a ganglioside biosynthesis inhibitor. In one embodiment, the disease or condition mediated by GalNAc transferase deficiency is tumorous calcinosis. In one embodiment, the ganglioside biosynthesis inhibitor is an inhibitor of a β1,4-N-acetylgalactosaminyl transferase.

In certain embodiments, provided herein is a method of treating a disorder mediated by gangliosides by administering to an individual (e.g., a human) in need thereof a therapeutically effective amount of any ganglioside synthesis inhibitor described herein. In specific embodiments, the ganglioside synthesis inhibitor is a modulator (e.g., inhibitor or promoter) of synthesis of a ceramide-linked disaccharide (e.g. LacCer), the initiation of ganglioside synthesis (e.g. via inhibition of 1-O-acylceramide synthase), further modification of a linkage disaccharide (e.g., glucosylation, galactosylation, sialylation), glycan sulfation, glycan acetylation, glycan phosphorylation, or degradation of gangliosides. In certain instances, the disorder mediated by ganglioside is a cancer, a tumor, undesired angiogenesis (e.g., cancer, diabetic blindness, age-related macular degeneration, rheumatoid arthritis, or psoriasis), insufficient angiogenesis (e.g., coronary artery disease, stroke, or delayed wound healing), mucopolysaccharidosis, amyloidosis, a spinal cord injury, hypertriglyceridemia, inflammation, a wound, or the like. In some embodiments, provided herein is a method of treating cancer by administering to an individual (e.g., a human) in need thereof a therapeutically effective amount of any ganglioside synthesis inhibitor described herein. In some embodiments, provided herein is a method of treating a tumor by administering to an individual (e.g., a human) in need thereof a therapeutically effective amount of any ganglioside synthesis inhibitor described herein. In some embodiments, provided herein is a method of treating undesired angiogenesis by administering to an individual (e.g., a human) in need thereof a therapeutically effective amount of any ganglioside synthesis inhibitor described herein. In some embodiments, provided herein is a method of treating a lysosomal storage disease (e.g., MPS) by administering to an individual (e.g., a human) in need thereof a therapeutically effective amount of any ganglioside synthesis inhibitor described herein. In some embodiments, provided herein is a method of treating a amyloidosis, a spinal cord injury, hypertriglyceridemia, and/or inflammation by administering to an individual (e.g., a human) in need thereof a therapeutically effective amount of any ganglioside synthesis inhibitor described herein. In some embodiments, provided herein is a method of treating Salidosis, Tay Sachs, Sandhoff, GM₁ gangliosidosis, or Fabry disease by administering to an individual (e.g., a human) in need thereof a therapeutically effective amount of any ganglioside synthesis inhibitor described herein.

In some embodiments, provided herein is a method of treating cancer by administering to an individual (e.g., human) a therapeutically effective amount of any ganglioside synthesis inhibitor described herein. In some embodiments, the cancer is, by way of non-limiting example, pancreatic cancer, myeloma, ovarian cancer, hepatocellular cancer, breast cancer, colon carcinoma, renal cell carcinoma, carcinoma of the gut, lung or urogenital tract, or melanoma.

In some embodiments, provided herein is a method of treating an infectious or viral disease by administering to an individual (e.g., human) a therapeutically effective amount of any ganglioside synthesis inhibitor described herein. In some embodiments, the infectious or viral disease includes, by way of non-limiting example, herpes, diphtheria, papilloma virus, hepatitis, HIV, coronavirus, or adenovirus.

In some embodiments, the treatment of amyloidosis includes the treatment of Alzheimer's disease, Parkinson's disease, type-2 diabetes, Huntington's disease, spongiform encephalopathies (Creutzfeldt-Jakob, Kuru, Mad Cow), diabetic amyloidosis, Rheumatoid arthritis, juvenile chronic arthritis, Ankylosing spondylitis, psoriasis, psoriatic arthritis, adult still disease, Becet syndrome, familial Mediterranean fever, Crohn's disease, leprosy, osteomyelitis, tuberculosis, chronic bronchiectasis, Castleman disease, Hodgkin's disease, renal cell carcinoma, carcinoma of the gut, lung or urogenital tract.

Provided in certain embodiments herein is a process of inhibiting ganglioside function in a cell comprising contacting the cell with a selective modulator (e.g., with respect to other glycans) of ganglioside biosynthesis. In various embodiments, ganglioside biosynthesis, as used herein, includes, by way of non-limiting example, (1) inhibition of (a) synthesis of a ceramide-linked disaccharide (e.g. LacCer), (b) further modification of a linkage disaccharide (e.g., glucosylation, galactosylation, sialylation), (c) glycan sulfation, glycan acetylation, and/or glycan phosphorylation, and/or (d) degradation of gangliosides.

In some embodiments, the modulator of ganglioside biosynthesis modulates (e.g., promotes or inhibits) synthesis of a ceramide-linked disaccharide (e.g. LacCer) and/or the initiation of ganglioside synthesis (e.g. via inhibition of 1-O-acylceramide synthase). In some embodiments, the modulator of ganglioside synthesis inhibits the synthesis of LacCer, the initiation of ganglioside synthesis, or a combination thereof. In some embodiments, modulators of ganglioside biosynthesis modulate (e.g., promote or inhibit) one or more of 1-O-acylceramide synthase, galactosylceramide synthase, glucosylceramide synthase and/or lactosylceramide synthase. In some embodiments, modulators of ganglioside biosynthesis modulate (e.g., promote or inhibit) synthesis of one or more of O, A, B or C gangliosides.

In some embodiments, the ganglioside synthesis inhibitor modulates (e.g., promote or inhibit) a glycosyltransferase. In some embodiments, the inhibitor of a ganglioside glycosyltransferase inhibits the synthesis of the linkage region (e.g. via linkage of ceramide to a lactosyl moiety), the initiation of ganglioside synthesis (e.g. via inhibition of 1-O-acylceramide synthase), the synthesis of ganglioside (e.g., further sialylation of a ceramide-linked glycan), or a combination thereof. In some embodiments, ganglioside biosynthesis inhibitors selectively modulate one or more of a glycosyltransferase, a glucosyl transferase or a sialyl transferase. In some embodiments, ganglioside biosynthesis inhibitors selectively modulate (e.g., promote or inhibit) synthesis of one or more of O, A, B or C gangliosides. In specific embodiments, the modulator of ganglioside biosynthesis inhibits synthesis of GT_{1b} gangliosides. In specific embodiments, the modulator of ganglioside biosynthesis promotes synthesis of GT_{1b} gangliosides. In specific embodiments, the modulator of ganglioside biosynthesis inhibits β-galactoside α-2,3-sialyltransferase, GlcNAcβ 1,3-galactosyltransferase, polypeptide 4, β-1,4-N-acetyl-galactosaminyl transferase 1, α-N-acetyl-neuraminide α-2,8-sialyltransferase 1, or β-galactoside α-2,3-sialyltransferase 5. In specific embodiments, the modulator of ganglioside biosynthesis promotes β-galactoside α-2,3-sialyltransferase, GlcNAcβ 1,3-galactosyltransferase, polypeptide 4, β-1,4-N-acetyl-galactosaminyl transferase 1, α-N-acetyl-neuraminide α-2,8-sialyltransferase 1, or β-galactoside α-2,3-sialyltransferase 5.

In certain embodiments, the effective amount of the ganglioside synthesis inhibitor alters or disrupts the nature (e.g., alters or disrupts the sialylation, glycosylation, concentration of ganglioside, chain length of ganglioside, or a combination thereof) of ganglioside compared to endogenous ganglioside in an amount sufficient to alter or disrupt ganglioside binding, ganglioside signaling, or a combination thereof. In specific embodiments, the ganglioside synthesis inhibitor described herein alters or disrupts the nature of the ganglioside (e.g., nature of O, A, B or C gangliosides) such that it inhibits ganglioside signaling. In other specific embodiments, the ganglioside synthesis inhibitor described herein alters or disrupts the nature of the ganglioside such that it inhibits ganglioside binding. In specific embodiments, the ganglioside synthesis inhibitor described herein alters or disrupts the nature of the ganglioside (e.g., nature of O, A, B or C gangliosides) such that it inhibits ganglioside binding. In more specific embodiments, the ganglioside synthesis inhibitor described herein alters or disrupts the nature of the ganglioside such that it inhibits ganglioside binding and ganglioside signaling. In specific embodiments, the ganglioside synthesis inhibitor described herein alters or disrupts the nature of the ganglioside (e.g., nature of O, A, B or C gangliosides) such that it inhibits ganglioside signaling and binding. In some embodiments, the ganglioside synthesis inhibitor alters or disrupts the nature of the ganglioside such that it inhibits the binding, signaling, or a combination thereof of any lectin (including polypeptides) subject to ganglioside binding, signaling or a combination thereof, in the absence of a ganglioside synthesis inhibitor. In some embodiments, the polypeptide is, by way of non-limiting example, a cell adhesion molecule (CAM). In certain embodiments, the CAM is an exogenous CAM, e.g., a bacterial lectin. In certain embodiments, the CAM is an endogenous CAM and includes, by way of non-limiting examples, E-selectin, L-selectin or P-selectin.

In certain embodiments, the selective modulator of ganglioside biosynthesis is a small molecule organic compound. In certain instances, selective modulator of ganglioside biosynthesis utilized herein is not a polypeptide or a carbohydrate. In certain embodiments, the small molecule organic compound has a molecular weight of less than about 2,000 g/mol, less than about 1,500 g/mol, less than about 1,000 g/mol, or less than about 500 g/mol.

Provided in certain embodiments herein is a method of treating cancer or neoplasia comprising administering a therapeutically effective amount of a ganglioside synthesis inhibitor to an individual in need thereof. In some embodiments, the ganglioside synthesis inhibitor reduces or inhibits tumor growth, reduces or inhibits angiogenesis, or a combination thereof. In certain embodiments, the ganglioside synthesis inhibitor alters or disrupts the GM₃:GD₃ ratio of gangliosides on a cell, tissue, organ or individual compared to endogenous GM₃:GD₃ ratio of ganglioside in an organism, organ, tissue or cell. In certain embodiments, the ganglioside synthesis inhibitor is a selective (as compared to other glycans) modulator of the synthesis of the linkage region (e.g. via linkage of ceramide to a lactosyl moiety), the initiation of ganglioside synthesis (e.g. via inhibition of 1-O-acylceramide synthase), the synthesis of ganglioside (e.g., further sialylation of a ceramide-linked glycan), or a combination thereof.

In various embodiments, a ganglioside synthesis inhibitor described herein alters or reduces the function of ganglioside by one or more of the following non-limiting manners: In various embodiments, a ganglioside biosynthesis inhibitor, as described herein, is a selective inhibitor of (a) synthesis of a ceramide-linked disaccharide (e.g. LacCer), (b) further modification of a linkage disaccharide (e.g., glucosylation, galactosylation, sialylation), (c) glycan sulfation, glycan acetylation, and/or glycan phosphorylation, and/or (d) degradation of gangliosides.

In some embodiments, the modulator of ganglioside biosynthesis modulates (e.g., promotes or inhibits) synthesis of a ceramide-linked disaccharide (e.g. LacCer). In some embodiments, the modulator of ganglioside synthesis inhibits the synthesis of LacCer, the initiation of ganglioside synthesis, or a combination thereof. In some embodiments, modulators of ganglioside biosynthesis modulate (e.g., promote or inhibit) one or more of 1-O-acylceramide synthase, galactosylceramide synthase, or glucosylceramide synthase.

In some embodiments, the ganglioside synthesis inhibitor modulates (e.g., promote or inhibit) a glycosyltransferase. In some embodiments, the inhibitor of a ganglioside glycosyltransferase inhibits the synthesis of the linkage region (e.g. via linkage of ceramide to a lactosyl moiety), the initiation of ganglioside synthesis (e.g. via inhibition of 1-O-acylceramide synthase), the synthesis of ganglioside (e.g., further sialylation of a ceramide-linked glycan), or a combination thereof. In some embodiments, ganglioside biosynthesis inhibitors selectively modulate one or more of a glycosyltransferase, a glucosyl transferase or a sialyl transferase. In some embodiments, ganglioside biosynthesis inhibitors selectively modulate (e.g., promote or inhibit) synthesis of one or more of O, A, B or C gangliosides. In specific embodiments, ganglioside synthesis inhibitors modulate one or more of a GT_{1b} synthase including, a GM₃ synthase (e.g., sialyl transferase I), a GM₂/GD₂ synthase (e.g., β1-4 GalNAc transferase), a GD₃ synthase (e.g. sialyl transferase II), Gal TII, or Sialyl TIV, as compared to a GM₁ synthase, a GD_{1aα} synthase, a GT_{1aα} synthase, or a GT_{1c} synthase. In specific embodiments, the modulator of ganglioside biosynthesis inhibits synthesis of GT_{1b} gangliosides. In specific embodiments, the modulator of ganglioside biosynthesis promotes synthesis of GT_{1b} gangliosides. In specific embodiments, the modulator of ganglioside biosynthesis inhibits β-galactoside α-2,3-sialyltransferase, GlcNAc β 1,3-galactosyltransferase, polypeptide 4, β-1,4-N-acetyl-galactosaminyl transferase 1, α-N-acetyl-neuraminide α-2,8-sialyltransferase 1, or β-galactoside α-2,3-sialyltransferase 5. In specific embodiments, the modulator of ganglioside biosynthesis promotes β-galactoside α-2,3-sialyltransferase GlcNAc β 1,3-galactosyltransferase, polypeptide 4, β-1,4-N-acetyl-galactosaminyl transferase 1, α-N-acetyl-neuraminide α-2,8-sialyltransferase 1, or β-galactoside α-2,3-sialyltransferase 5.

In some embodiments, modulation of ganglioside biosynthesis includes the inhibition of the addition of a NeuNAc residue to a ganglioside having the structure:

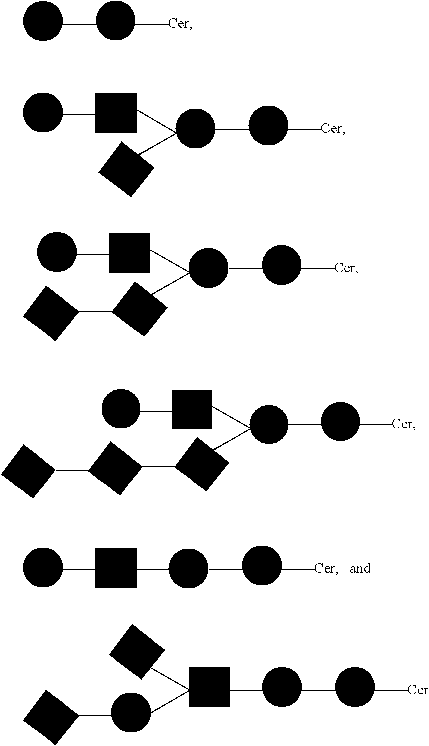

via an α2,3 linkage
wherein: [symbol]
is a galactose residue; [symbol]
is a glucose residue; [symbol]
is an N-acetylgalactosamine residue; [symbol]
is a NeuNAc residue; and
Cer is ceramide

In one embodiment the selective inhibitor of ganglioside biosynthesis inhibits the addition of a NeuNAc residue to a ganglioside having the structure:

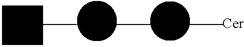

via an α2,3 linkage;
wherein: [symbol]
is a galactose residue; [symbol]
is a glucose residue; [symbol]
is an N-acetylgalactosamine residue; and
Cer is ceramide

In another embodiment the selective inhibitor of ganglioside biosynthesis inhibits the addition of a NeuNAc residue to a ganglioside having the structure:

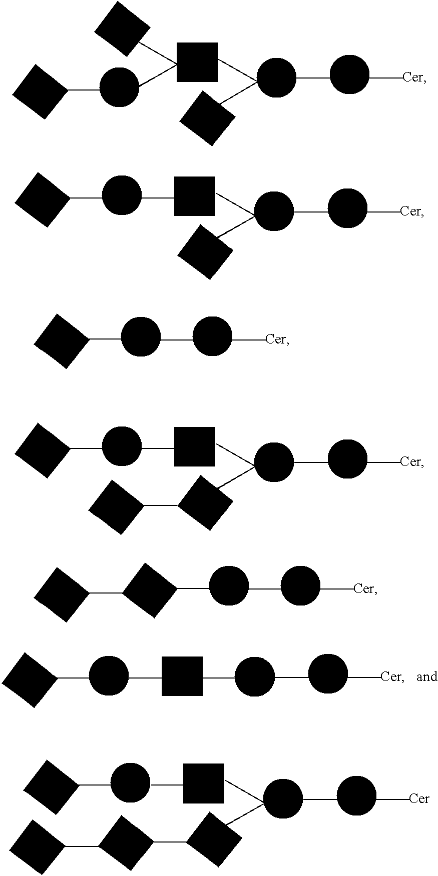

via an α2,8 linkage;
wherein: [symbol]
is a galactose residue; [symbol]
is a glucose residue; [symbol]
is an N-acetylgalactosamine residue; [symbol]
is a NeuNAc residue; and
Cer is ceramide

In yet another embodiment the selective inhibitor of ganglioside biosynthesis inhibits the addition of an N-acetylgalactosamine residue to a ganglioside having the structure:

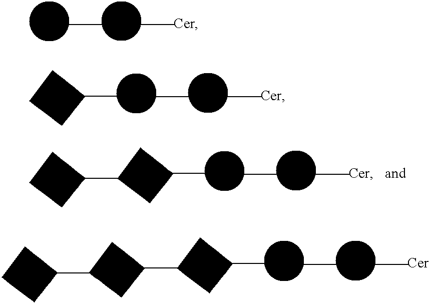

via an β1,4 linkage;
wherein: [symbol]
is a galactose residue; [symbol]
is a glucose residue; [symbol]
is an N-acetylgalactosamine residue; [symbol]
is a NeuNAc residue; and
Cer is ceramide

In a further embodiment the selective inhibitor of ganglioside biosynthesis inhibits the addition of a galactose residue to a ganglioside having the structure:

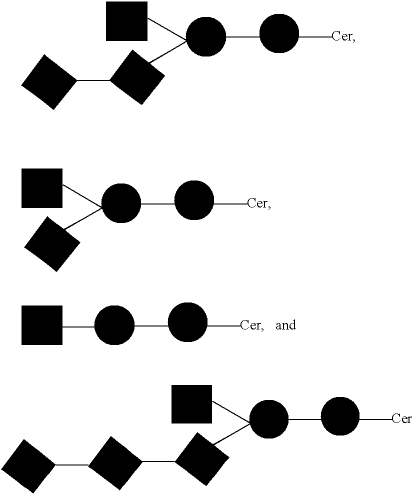

via an β1,3 linkage;
wherein: [symbol]
is a galactose residue; [symbol]
is a glucose residue; [symbol]
is an N-acetylgalactosamine residue; [symbol]
is a NeuNAc residue; and
Cer is ceramide

In certain embodiments, the effective amount of the ganglioside synthesis inhibitor alters or disrupts the nature (e.g., alters or disrupts the sialylation, glycosylation, acetylation, sulfation, O-sulfation, the 3-O sulfation, concentration of ganglioside, chain length of ganglioside, or a combination thereof) of ganglioside compared to endogenous ganglioside in an amount sufficient to alter or disrupt ganglioside binding, ganglioside signaling, or a combination thereof. In specific embodiments, the ganglioside synthesis inhibitor described herein alters or disrupts the nature of the ganglioside (e.g., nature of O, A, B or C gangliosides) such that it inhibits ganglioside signaling. In other specific embodiments, the ganglioside synthesis inhibitor described herein alters or disrupts the nature of the ganglioside such that it inhibits ganglioside binding. In specific embodiments, the ganglioside synthesis inhibitor described herein alters or disrupts the nature of the ganglioside (e.g., nature of O, A, B or C gangliosides) such that it inhibits ganglioside binding. In more specific embodiments, the ganglioside synthesis inhibitor described herein alters or disrupts the nature of the ganglioside such that it inhibits ganglioside binding and ganglioside signaling. In specific embodiments, the ganglioside synthesis inhibitor described herein alters or disrupts the nature of the ganglioside (e.g., nature of O, A, B or C gangliosides) such that it inhibits ganglioside signaling and binding. In some embodiments, the ganglioside synthesis inhibitor alters or disrupts the nature of the ganglioside such that it inhibits the binding, signaling, or a combination thereof of any lectin (including polypeptides) subject to ganglioside binding, signaling or a combination thereof, in the absence of a ganglioside synthesis inhibitor. In some embodiments, the polypeptide is, by way of non-limiting example, a cell adhesion molecule (CAM). In certain embodiments, the CAM is an exogenous CAM, e.g., a bacterial lectin. In certain embodiments, the CAM is an endogenous CAM and includes, by way of non-limiting examples, E-selectin, L-selectin or P-selectin.

In certain embodiments, ganglioside synthesis inhibitors described herein are small molecule organic compounds. In certain instances, ganglioside synthesis inhibitors utilized herein are not polypeptides or carbohydrates. In some embodiments, a small molecule organic compound has a molecular weight of less than 2,000 about g/mol, less than 1,500 about g/mol, less than about 1,000 g/mol, or less than about 500 g/mol.

Provided in some embodiments herein is a method of treating a lysosomal storage disease comprising administering a therapeutically effective amount of a ganglioside synthesis inhibitor to an individual (e.g., a human) in need thereof. In certain embodiments, the ganglioside synthesis inhibitor is a selective (as compared to other glycans) inhibitor of ganglioside synthesis. In some embodiments, the selective ganglioside synthesis inhibitor is a selective modulator (e.g., inhibitor or promoter) of (a) synthesis of a ceramide-linked disaccharide (e.g. LacCer), (b) further modification of a linkage disaccharide (e.g., glucosylation, galactosylation, sialylation), (c) glycan sulfation, glycan acetylation, and/or glycan phosphorylation, and/or (d) degradation of gangliosides.

In specific embodiments, the lysosomal storage disease is, by way of non-limiting example, mucopolysaccharidosis (MPS). In more specific embodiments, the MPS is, by way of non-limiting example, MPS I, MPS II or MPS III. In some embodiments, a lysosomal storage disease is a glycolipid storage disease. In some embodiments, a glycolipid storage disease is, by way of non-limiting example, Salidosis, Tay Sachs, Sandhoff, GM₁ gangliosidosis, or Fabry disease.

In various embodiments, a ganglioside synthesis inhibitor described herein alters or reduces the function of ganglioside by one or more of the following non-limiting manners: In various embodiments, a ganglioside biosynthesis inhibitor, as described herein, is (1) a selective inhibitor of (a) synthesis of a ceramide-linked disaccharide (e.g. LacCer), (b) further modification of a linkage disaccharide (e.g., glucosylation, galactosylation, sialylation), (c) glycan sulfation, glycan acetylation, and/or glycan phosphorylation, and/or (d) degradation of gangliosides.

In some embodiments, the modulator of ganglioside biosynthesis modulates (e.g., promotes or inhibits) synthesis of a ceramide-linked disaccharide (e.g. LacCer). In some embodiments, the modulator of ganglioside synthesis inhibits the synthesis of LacCer, the initiation of ganglioside synthesis, or a combination thereof. In some embodiments, modulators of ganglioside biosynthesis modulate (e.g., promote or inhibit) one or more of 1-O-acylceramide synthase, galactosylceramide synthase, or glucosylceramide synthase.

In some embodiments, the ganglioside synthesis inhibitor modulates (e.g., promote or inhibit) a glycosyltransferase. In some embodiments, the inhibitor of a ganglioside glycosyltransferase inhibits the synthesis of the linkage region (e.g. via linkage of ceramide to a lactosyl moiety), the initiation of ganglioside synthesis (e.g. via inhibition of 1-O-acylceramide synthase), the synthesis of ganglioside (e.g., further sialylation of a ceramide-linked glycan), or a combination thereof. In some embodiments, ganglioside biosynthesis inhibitors selectively modulate one or more of a glycosyltransferase, a glucosyl transferase or a sialyl transferase. In some embodiments, ganglioside biosynthesis inhibitors selectively modulate (e.g., promote or inhibit) synthesis of one or more of O, A, B or C gangliosides. In specific embodiments, ganglioside synthesis inhibitors modulate one or more of a GT_{1b} synthase including, a GM₃ synthase (e.g., sialyl transferase I), a GM₂/GD₂ synthase (e.g., β1-4 GalNAc transferase), a GD₃ synthase (e.g. sialyl transferase II), Gal TII, or Sialyl TIV, as compared to a GM₁ synthase, a GD₁ synthase, a GT_{1aα} synthase, or a GT_{1c} synthase. In specific embodiments, the modulator of ganglioside biosynthesis inhibits synthesis of GT_{1b} gangliosides. In specific embodiments, the modulator of ganglioside biosynthesis promotes synthesis of GT_{1b} gangliosides. In specific embodiments, the modulator of ganglioside biosynthesis inhibits β-galactoside α-2,3-sialyltransferase, GlcNAcβ 1,3-galactosyltransferase, polypeptide 4, β-1,4-N-acetyl-galactosaminyl transferase 1, α-N-acetyl-neuraminide α-2,8-sialyltransferase 1, or β-galactoside α-2,3-sialyltransferase 5. In specific embodiments, the modulator of ganglioside biosynthesis promotes β-galactoside α-2,3-sialyltransferase, GlcNAcβ 1,3-galactosyltransferase, polypeptide 4, β-1,4-N-acetyl-galactosaminyl transferase 1, α-N-acetyl-neuraminide α-2,8-sialyltransferase 1, or β-galactoside α-2,3-sialyltransferase 5.

In certain embodiments, the effective amount of the ganglioside synthesis inhibitor alters or disrupts the nature (e.g., alters or disrupts the sialylation, glycosylation, acetylation, sulfation, O-sulfation, the 3-O sulfation, concentration of ganglioside, chain length of ganglioside, or a combination thereof) of ganglioside compared to endogenous ganglioside in an amount sufficient to alter or disrupt ganglioside binding, ganglioside signaling, or a combination thereof. In specific embodiments, the ganglioside synthesis inhibitor described herein alters or disrupts the nature of the ganglioside (e.g., nature of O, A, B or C gangliosides) such that it inhibits ganglioside signaling. In other specific embodiments, the ganglioside synthesis inhibitor described herein alters or disrupts the nature of the ganglioside such that it inhibits ganglioside binding. In specific embodiments, the ganglioside synthesis inhibitor described herein alters or disrupts the nature of the ganglioside (e.g., nature of O, A, B or C gangliosides) such that it inhibits ganglioside binding. In more specific embodiments, the ganglioside synthesis inhibitor described herein alters or disrupts the nature of the ganglioside such that it inhibits ganglioside binding and ganglioside signaling. In specific embodiments, the ganglioside synthesis inhibitor described herein alters or disrupts the nature of the ganglioside (e.g., nature of O, A, B or C gangliosides) such that it inhibits ganglioside signaling and binding. In some embodiments, the ganglioside synthesis inhibitor alters or disrupts the nature of the ganglioside such that it inhibits the binding, signaling, or a combination thereof of any lectin (including polypeptides) subject to ganglioside binding, signaling or a combination thereof, in the absence of a ganglioside synthesis inhibitor. In some embodiments, the polypeptide is, by way of non-limiting example, a cell adhesion molecule (CAM). In certain embodiments, the CAM is an exogenous CAM, e.g., a bacterial lectin. In certain embodiments, the CAM is an endogenous CAM and includes, by way of non-limiting examples, E-selectin, L-selectin or P-selectin.

In certain embodiments, ganglioside synthesis inhibitors described herein are small molecule organic compounds. In certain instances, ganglioside synthesis inhibitors utilized herein are not polypeptides or carbohydrates. In some embodiments, a small molecule organic compound has a molecular weight of less than about 2,000 g/mol, less than 1,500 about g/mol, less than about 1,000 g/mol, or less than about 500 g/mol.

### Screening Processes

Provided herein are processes for identifying inhibitors of the biosynthesis of gangliosides or for identifying genes involved in (including selective modulators, inhibitors or the like) the biosynthesis of gangliosides. Also provided herein are processes for identifying modulators of enzymes involved in the biosynthesis of gangliosides.

In one embodiment is a cell-based high throughput process for identifying and/or screening for (1) ganglioside biosynthesis inhibitors; (2) genes involved in (including selective regulators of) the biosynthesis of gangliosides; (3) ganglioside biosynthesis modulators; or (4) combinations thereof. In one embodiment, a library of small-molecule chemical compounds (including oligopeptides and oligonucleotides) is screened; in other embodiments, a library of siRNA is screened; in other embodiments, both types of libraries are simultaneously or sequentially screened.

In certain embodiments, the siRNA library is enzymatically generated; or rationally synthesized; or randomly generated; or a combination thereof. Non-limiting examples of protocols for screening siRNA libraries in high-throughput genetic screens is found in the Journal of Cancer Molecules: 1(1), 19-24, 2005.

Provided in some embodiments is a process for identifying a compound that modulates ganglioside biosynthesis comprising:
a. contacting a mammalian cell with the compound in combination with a labeled probe that binds one or more gangliosides;
b. incubating the mammalian cell, compound and labeled probe;
c. collecting the labeled probe that is bound to one or more gangliosides; and
d. detecting or measuring the amount of labeled probe bound to one or more gangliosides.

In more specific embodiments, provided herein is a process for identifying a compound that selectively modulates ganglioside biosynthesis comprising:
a. contacting a mammalian cell with the compound;
b. contacting the mammalian cell and compound combination with a first labeled probe and a second labeled probe, wherein the first labeled probe binds one or more gangliosides and the second labeled probe binds at least one glycan (e.g., a GAG, a sulfated GAG, an extracellular glycan, or the like) other than a ganglioside or specific type of targeted ganglioside (i.e., other than the one or more ganglioside) or a specific type of targeted ganglioside (i.e., other than the one or more ganglioside);
c. incubating the mammalian cell, compound, the first labeled probe, and the second labeled probe;
d. collecting the first labeled probe that is bound to one or more gangliosides;
e. collecting the second labeled probe that is bound to at least one glycan (e.g., a GAG, a sulfated GAG, an extracellular glycan, or the like) other than a ganglioside or specific type of targeted ganglioside (i.e., other than the one or more ganglioside) or a specific type of targeted ganglioside (i.e., other than the one or more ganglioside);
f. detecting or measuring the amount of first labeled probe bound to one or more gangliosides; and
g. detecting or measuring the amount of the second labeled probe bound to at least one glycan (e.g., a GAG, a sulfated GAG, an extracellular glycan, or the like) other than a ganglioside or a specific type of targeted ganglioside (i.e., other than the one or more ganglioside).

Similarly, in some embodiments provided herein is a process for identifying compounds that selectively modulate ganglioside biosynthesis comprising:
a. contacting a first mammalian cell with the compound;
b. contacting the first mammalian cell and compound combination with a first labeled probe, wherein the first labeled probe binds one or more gangliosides;
c. incubating the first mammalian cell, compound, the first labeled probe, and the second labeled probe;
d. collecting the first labeled probe that is bound to one or more gangliosides;
e. detecting or measuring the amount of first labeled probe bound to one or more gangliosides;
f. contacting a second mammalian cell with the compound, wherein the second mammalian cell is of the same type as the first mammalian cell;
g. contacting the second mammalian cell and compound combination with a second labeled probe, wherein the second labeled probe binds at least one glycan (e.g., a GAG, a sulfated GAG, an extracellular glycan, or the like) other than a ganglioside or specific type of targeted ganglioside (i.e., other than the one or more ganglioside);
h. collecting the second labeled probe that is bound to at least one glycan (e.g., a GAG, a sulfated GAG, an extracellular glycan, or the like) other than a ganglioside or a specific type of ganglioside (i.e., other than the one or more ganglioside); and
i. detecting or measuring the amount of the second labeled probe bound to at least one glycan (e.g., a GAG, a sulfated GAG, an extracellular glycan, or the like) other than a ganglioside or specific type of targeted ganglioside (i.e., other than the one or more ganglioside).

In some embodiments, provided herein is a process for identifying a compound that modulates ganglioside biosynthesis comprising:
a. collecting gangliosides from a first mammalian cell of a selected type, wherein the ganglioside is a O series, A series, B series or C series ganglioside;
b. cleaving the gangliosides into a plurality of monosaccharide, disaccharide or oligosaccharide component parts;
c. detecting or measuring the amount of one or more of the monosaccharide, disaccharide or oligosaccharide component parts;
d. contacting and incubating a second mammalian cell of the selected type with the compound;
e. collecting gangliosides from the second mammalian cell of a selected type;
f. cleaving the gangliosides into a plurality of monosaccharide, disaccharide or oligosaccharide component parts;
g. detecting or measuring the amount of one or more of the monosaccharide, disaccharide or oligosaccharide component parts;
h. comparing:
   i. the amounts of gangliosides produced by the first and second mammalian cells;
   ii. the amounts of monosaccharides, disaccharides or oligosaccharides characteristic of O, A, B and/or C series gangliosides produced from the first and second mammalian cells;
   iii. the relative amounts of monosaccharides, disaccharides or oligosaccharides characteristic of O, A, B and/or C series gangliosides produced from the first and second mammalian cell;
   iv. the amounts of sialic acid residues produced by the first and second mammalian cells; or
   v. a combination thereof.

In some embodiments, monosaccharides, disaccharides or oligosaccharides characteristic of O, A, B or C series gangliosides that are provided by cleaving the gangliosides are components of GT_{1b}, GM₁ₐ, GM₂, GM₃, GD₃, GT_{1c}, GA₂, GA₁, GM_{1b}, GD_{1c}, GD₁ₐ, GT₁ₐ, GD₂, GD_{1b}, GQ_{1b}, GT₃, GT₂, GT₁ₐ, GQ_{1c}, GP_{1c} gangliosides or the like. In some embodiments, the amount of any one specific ganglioside (e.g., a GT_{1b}, GM₁ₐ, GM₂, GM₃, GD₃, GT_{1c}, GA₂, GA₁, GM_{1b}, GD_{1c}, GD₁ₐ, GT₁ₐ, GD₂, GD_{1b}, GQ_{1b}, GT₃, GT₂, GT₁ₐ, GQ_{1c}, or GP_{1c} ganglioside or the like, or the one or more component part thereof) collected from a first mammalian cell is compared to the amount of any other specific type of ganglioside, or the one or more component part thereof, produced by a second mammalian cell. In some embodiments, the amount of one or more specific gangliosides (e.g., one or more of GT_{1b}, GM₁ₐ, GM₂, GM₃, GD₃, GT_{1c}, GA₂, GA₁, GM_{1b}, GD_{1c}, GD₁ₐ, GT₁ₐ, GD₂, GD_{1b}, GQ_{1b}, GT₃, GT₂, GT₁ₐ, GQ_{1c}, or GP_{1c} gangliosides or the like, or the one or more component part thereof) produced by a first mammalian cell is compared to the amount of one or more of any other specific type of ganglioside or the total amount of gangliosides, or the one or more component part thereof, produced by a second mammalian cell.

In some embodiments, incubating the mixture of the compound with the at least one cell expressing at least one ganglioside is performed for a predetermined time. In one embodiment, incubation is for a period of about 12 hours. In another embodiment, incubating the mixture is for a period of about 18 hours. In another embodiment, about 24 hours. In yet another embodiment, about 36 hours. In a further embodiment, 48 hours. In another embodiment, at least about 12 hours, at least about 24 hours, at least about 36 hours, at least about 2 days, at least about 3 days, at least about 4 days, at least about 5 days, at least about 6 days, or at least about 7 days.

In one embodiment, the process(es) described herein are useful for high-throughput analysis of a ganglioside biosynthesis inhibitor or a positive or negative regulatory gene for ganglioside biosynthesis. In certain embodiments, the amounts of gangliosides and/or monosaccharides, disaccharides or oligosaccharides characteristic of O, A, B or C series gangliosides are measured with an analytical device. In some embodiments, the analytical device is a fluorometer. In certain embodiments, the analytical device is a fluorescent plate reader. In some embodiments, fluorescence is measured at any suitable excitation (e.g., of about 400 nm to about 600 nm) and any suitable emission (e.g., of about 500 nm to about 750 nm). In some embodiments, the detecting or measuring process is developed using a robotic pipetter.

In some embodiments, the process further comprises comparing the amount of first labeled probe bound to gangliosides to the amount of the second labeled probe bound to at least one glycan other than a ganglioside or specific type of targeted ganglioside (i.e., other than the one or more ganglioside) (e.g., to determine a ratio of the amount of first labeled probe bound to the amount of second labeled probe bound under substantially similar conditions).

In certain embodiments, a label utilized in any process described herein is any suitable label such as, by way of non-limiting example, a fluorescent label, a dye, a radiolabel, or the like. In some embodiments, the labeled probe comprises a biotinyl moiety and the process further comprises tagging the labeled probe with streptavidin-Cy5-PE. In certain embodiments, the first probe is any ganglioside binding protein, e.g., EGFR, NGFR, Cholera Toxin B-subunit (CTB), tetanus toxin (TTx), or the like. In various embodiments, the amount of bound labeled probes are detected in any suitable manner, e.g., with a fluorimeter, a radiation detector, or the like.

In certain embodiments, the first and second probes are labeled in a manner so as to be independently detectable. In some embodiments, the first and second probes are contacted to the cells separately (i.e., to different cells of the same type) and independently analyzed. In some embodiments, the at least one glycan (e.g., a GAG, a sulfated GAG, an extracellular glycan, or the like) other than a ganglioside or specific type of targeted ganglioside (i.e., other than the one or more ganglioside) is, by way of non-limiting example, chondroitin sulfate, O-linked glycans, N-linked glycans, heparan sulfate or the like. Furthermore, in some embodiments, a third labeled probe that binds at least one glycan (e.g., a GAG, a sulfated GAG, an extracellular glycan, or the like) not bound by the first or second labeled probe is also utilized. Additional labeled probes are also optionally utilized.

Second and additional labeled probes include any labeled compound or labeled lectin suitable (e.g., a labeled compound or lectin that binds a GAG, a non-ganglioside glycan, a non-sulfated GAG, an extracellular glycan, an O-linked glycan, an N-linked glycan, chondroitin sulfate, dermatan sulfate, keratin sulfate, and/or hyaluronan). In some embodiments, labeled probes included labeled forms of one or more of, by way of non-limiting example, Wheat Germ Agglutinin (WGA) from Triticum vulgaris (as a probe for binding N-linked and O-linked glycans with terminal GlcNAc residues and clustered sialic acid residues); Phaseolus Vulgaris Aggutinin (PHA) from Phaseolus vulgaris (as a probe for binding N-linked glycans); Cholera Toxin B-subunit (CTB) from Vibrio cholera (as a probe for binding sialic acid modified glycolipids); Concanavalin A (ConA) from Canavalia ensiformis (as a probe for binding mannose residues in N-linked glycans); and/or Jacalin from Artocarpus integrifolia (as a probe for binding O-linked glycans). In specific embodiments, labeled forms of each of Wheat Germ Agglutinin (WGA) from Triticum vulgaris (as a probe for binding N-linked and O-linked glycans with terminal GlcNAc residues and clustered sialic acid residues); Phaseolus Vulgaris Aggutinin (PHA) from Phaseolus vulgaris (as a probe for binding N-linked glycans); and Cholera Toxin B-subunit (CTB) from Vibrio cholera (as a probe for binding sialic acid modified glycolipids) are utilized.

Contact with first, second and additional labeled probes occurs in parallel, concurrently, or sequentially. In certain embodiments, contacting the compounds and multiple probes allows identification of selective ganglioside inhibitors.

In some embodiments, the mammalian cell (e.g., human cell) is selected from any suitable mammalian cell. In specific embodiments, the mammalian cell is, by way of non-limiting example, a human cancer cell (e.g., human cervical cancer cell (HeLa)), a human ovarian cancer cell (SKOV), a human lung cancer cell (Hal8), a human medulloblastoma cancer cell (DAOY), a Chinese Hamster Ovary (CHO) cell, an adenocarcinoma cell, a melanoma cell, or a human primary cell. In certain embodiments, included herein are processes wherein the cell includes a plurality (e.g., 2, 3, 4 or all) of a human cancer cell (e.g., human cervical cancer cell (HeLa)), a human ovarian cancer cell (SKOV), a human lung cancer cell (Hal8), a human medulloblastoma cancer cell (DAOY), and/or a Chinese Hamster Ovary (CHO) cell. Contact with such cells optionally occurs in parallel, concurrently, or sequentially. In certain embodiments, contact with multiple cells identifies ganglioside inhibitors (e.g., selective ganglioside inhibitors) that inhibit ganglioside biosynthesis in multiple cell lines. In some instances, utilization of a plurality of cell lines allows the elimination or minimization of false positives in identifying ganglioside inhibitors.

Thus, in some embodiments, any process described herein comprises contacting the compound to a first cell (type), contacting the compound to a second cell (type), and, optionally, contacting the compound to additional cells (types), and repeating the process described for each of the first, second and any additional cell types utilized (e.g., to determine if a ganglioside inhibitor is selective for multiple cell lines or to determine which types of cell lines that the ganglioside inhibitor selectively targets). Furthermore, in such embodiments, the process further comprises comparing the amount of labeled probe (or the amount of first, second or any additional labeled probe) that is bound in each type of cell (e.g., to determine selectivity of inhibiting ganglioside biosynthesis compared to the biosynthesis of other types of glycans).

In some embodiments, once a compound that modulates ganglioside biosynthesis is determined by the process described, a similar process is optionally utilized to determine whether or not the compound selectively modulates ganglioside biosynthesis. Specifically, selectivity of a compound that modulates ganglioside biosynthesis is determined by utilizing a similar process as described for determining whether or not the compound modulates ganglioside biosynthesis, e.g., by:
a. contacting a mammalian cell with the compound in combination with a labeled probe that binds one or more non-ganglioside glycan (e.g., GAG or other class of glycan);
b. incubating the mammalian cell, compound and labeled probe;
c. collecting the labeled probe that is bound to non-ganglioside glycan (e.g., GAG or other class of glycan); and
d. detecting or measuring the amount of labeled probe bound to non-ganglioside glycan (e.g., GAG or other class of glycan).

In various embodiments, this process is repeated for any number of non-ganglioside glycans (e.g., GAG or other class of glycan). In some embodiments, the non-ganglioside glycans are, by way of non-limiting example, chondroitin sulfate, heparan sulfate, O-linked glycans, N-linked glycans, or the like.

In some embodiments, the mammalian cell (e.g., human cell) is selected from any suitable mammalian cell. In specific embodiments, the mammalian cell is, by way of non-limiting example, a human cancer cell (e.g., human cervical cancer cell (HeLa)) a human ovarian cancer cell (SKOV), a human lung cancer cell (Hal8), a human medulloblastoma cancer cell (DAOY) or a human primary cell. Furthermore, in some embodiments, the process is repeated utilizing one or more additional cell types. In certain embodiments, the results (e.g., of (c), and/or (d)) from the one or more additional cell types (e.g., a second, third, fourth, fifth or the like cell types) are compared to each other and the results (e.g., of (c), and/or (d)) from the first cell type.

In certain embodiments, the gangliosides and/or the modified gangliosides are cleaved in any suitable manner. In some embodiments, the gangliosides and/or the modified gangliosides are cleaved using a suitable enzyme such as endoglyceramidase, or in any other suitable chemical manner.

In some embodiments, the amount of monosaccharide, disaccharide or oligosaccharide units present in the cell and/or the characteristic of the gangliosides in a cell are determined in any suitable manner. For example, in some embodiments, the ratios of O, A, B and/or C series gangliosides and/or the amount of sialic acid units and/or the amount of O-sulfation (e.g., 3-O-sulfation) of the glucosylamine groups present in the gangliosides, or a combination thereof is determined utilizing a carbazole assay, high performance liquid chromatography (HPLC), thin layer chromatography (TLC), capillary electrophoresis, gel electrophoresis, mass spectrum (MS) analysis, HPLC electrospray ionization tandem mass spectrometry, nuclear magnetic resonance (NMR) analysis, or the like.

In certain embodiments, a process described herein is a process for identifying compounds that selectively modulate ganglioside biosynthesis. In such embodiments, the process also comprises collecting one or more non-ganglioside glycan (e.g., a sulfated glycan, such as chondroitin sulfate, O-linked glycans, N-linked glycans, or the like) from the cell, both without incubation with the compound and with incubation with the compound; cleaving each of such non-ganglioside glycans; measuring the character of each of such non-ganglioside glycan; and comparing the character of the non-ganglioside glycan that was not incubated with the character of the non-ganglioside glycan that was incubated. In certain embodiments, the character includes, by way of non-limiting example, the chain length of the non-ganglioside glycan, the amount of sulfation of the non-ganglioside glycan, the location of sulfation of the non-ganglioside glycan, the structure of the non-ganglioside glycan, the composition of the non-ganglioside glycan, or the like. The structure of glycosaminoglycans, N-linked glycans, O-linked glycans, and lipid linked glycans can be determined using any suitable method, including, by way of non-limiting example, monosaccharide compositional analysis, capillary electrophoresis, gel electrophoresis, gel filtration, high performance liquid chromatography (HPLC), thin layer chromatography (TLC), mass spectrum (MS) analysis, HPLC electrospray ionization tandem mass spectrometry, nuclear magnetic resonance (NMR) analysis, or the like.

### Combinations

In certain instances, it is appropriate to administer at least one therapeutic compound described herein (i.e., any ganglioside synthesis inhibitor described herein) in combination with another therapeutic agent. By way of example only, if one of the side effects experienced by a patient upon receiving one of the ganglioside synthesis inhibitors described herein is nausea, then it is appropriate in certain instances to administer an anti-nausea agent in combination with the initial therapeutic agent. Or, by way of example only, the therapeutic effectiveness of one of the ganglioside synthesis inhibitors described herein is enhanced by administration of an adjuvant (i.e., by itself the adjuvant has minimal therapeutic benefit, but in combination with another therapeutic agent, the overall therapeutic benefit to the patient is enhanced). Or, by way of example only, the benefit experienced by a patient is increased by administering one of ganglioside synthesis inhibitors described herein with another therapeutic agent (which also includes a therapeutic regimen) that also has therapeutic benefit. In any case, regardless of the disease, disorder or condition being treated, the overall benefit experienced by the patient is in some embodiments additive of the two therapeutic agents or in other embodiments, the patient experiences a synergistic benefit.

In some embodiments, the particular choice of compounds depends upon the diagnosis of the attending physicians and their judgment of the condition of the patient and the appropriate treatment protocol. The compounds are optionally administered concurrently (e.g., simultaneously, essentially simultaneously or within the same treatment protocol) or sequentially, depending upon the nature of the disease, disorder, or condition, the condition of the patient, and the actual choice of compounds used. In certain instances, the determination of the order of administration, and the number of repetitions of administration of each therapeutic agent during a treatment protocol, is based on an evaluation of the disease being treated and the condition of the patient.

In some embodiments, therapeutically-effective dosages vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the patient.

In some embodiments of the combination therapies described herein, dosages of the co-administered compounds vary depending on the type of co-drug employed, on the specific drug employed, on the disease or condition being treated and so forth. In addition, when co-administered with one or more biologically active agents, the compound provided herein is optionally administered either simultaneously with the biologically active agent(s), or sequentially. In certain instances, if administered sequentially, the attending physician will decide on the appropriate sequence of therapeutic compound described herein in combination with the additional therapeutic agent.

The multiple therapeutic agents (at least one of which is a ganglioside synthesis inhibitor described herein) are optionally administered in any order or even simultaneously. If simultaneously, the multiple therapeutic agents are optionally provided in a single, unified form, or in multiple forms (by way of example only, either as a single pill or as two separate pills). In certain instances, one of the therapeutic agents is optionally given in multiple doses. In other instances, both are optionally given as multiple doses. If not simultaneous, the timing between the multiple doses is any suitable timing, e.g., from more than zero weeks to less than four weeks. In some embodiments, the additional therapeutic agent is utilized to achieve remission (partial or complete) of a cancer, whereupon the therapeutic agent described herein (e.g., any ganglioside synthesis inhibitor) is subsequently administered. In addition, the combination methods, compositions and formulations are not to be limited to the use of only two agents; the use of multiple therapeutic combinations is also envisioned (including two or more therapeutic compounds described herein).

In certain embodiments, a dosage regimen to treat, prevent, or ameliorate the condition(s) for which relief is sought, is modified in accordance with a variety of factors. These factors include the disorder from which the subject suffers, as well as the age, weight, sex, diet, and medical condition of the subject. Thus, in various embodiments, the dosage regimen actually employed varies and deviates from the dosage regimens set forth herein.

In some embodiments, the pharmaceutical agents which make up the combination therapy disclosed herein are provided in a combined dosage form or in separate dosage forms intended for substantially simultaneous administration. In certain embodiments, the pharmaceutical agents that make up the combination therapy are administered sequentially, with either therapeutic compound being administered by a regimen calling for two-step administration. In some embodiments, two-step administration regimen calls for sequential administration of the active agents or spaced-apart administration of the separate active agents. In certain embodiments, the time period between the multiple administration steps varies, by way of non-limiting example, from a few minutes to several hours, depending upon the properties of each pharmaceutical agent, such as potency, solubility, bioavailability, plasma half-life and kinetic profile of the pharmaceutical agent.

In addition, the ganglioside synthesis inhibitors described herein also are optionally used in combination with procedures that provide additional or synergistic benefit to the patient. By way of example only, patients are expected to find therapeutic and/or prophylactic benefit in the methods described herein, wherein pharmaceutical composition of a compound disclosed herein and/or combinations with other therapeutics are combined with genetic testing to determine whether that individual is a carrier of a gene or gene mutation that is known to be correlated with certain diseases or conditions.

In various embodiments, the ganglioside synthesis inhibitors described herein and combination therapies are administered before, during or after the occurrence of a disease or condition. Timing of administering the composition containing a ganglioside synthesis inhibitor is optionally varied to suit the needs of the individual treated. Thus, in certain embodiments, the ganglioside synthesis inhibitors are used as a prophylactic and are administered continuously to subjects with a propensity to develop conditions or diseases in order to prevent the occurrence of the disease or condition. In some embodiments, the compounds and compositions are administered to a subject during or as soon as possible after the onset of the symptoms. The administration of the ganglioside synthesis inhibitors are optionally initiated within the first 48 hours of the onset of the symptoms, within the first 6 hours of the onset of the symptoms, or within 3 hours of the onset of the symptoms. The initial administration is achieved by any route practical, such as, for example, an intravenous injection, a bolus injection, infusion over 5 minutes to about 5 hours, a pill, a capsule, transdermal patch, buccal delivery, and the like, or combination thereof. In some embodiments, the compound should be administered as soon as is practicable after the onset of a disease or condition is detected or suspected, and for a length of time necessary for the treatment of the disease, such as, for example, from about 1 month to about 3 months. The length of treatment is optionally varied for each subject based on known criteria. In exemplary embodiments, the compound or a formulation containing the compound is administered for at least 2 weeks, between about 1 month to about 5 years, or from about 1 month to about 3 years.

In certain embodiments, therapeutic agents are combined with or utilized in combination with one or more of the following therapeutic agents in any combination: therapeutic agent for treating lysosomal storage disease (LSD), Imiglucerase (Cerazyme), laronidase (Aldurazyme), idursulfase (Elaprase), galsulfase (Naglazyme), agalsidase beta (Fabrazyme), alglucosidase alfa (Myozyme), agalsidase alfa (Replagal), miglustat (Zavesca), Genz-112638, anti-inflammatory agents, immunosuppressants or anti-cancer therapies (e.g., radiation, surgery, or anti-cancer agents).

In some embodiments, one or more of the anti-cancer agents are proapoptotic agents. Examples of anti-cancer agents include, by way of non-limiting example: gossypol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, or PD 184352, Taxol™, also referred to as “paclitaxel”, is an anti-cancer drug which acts by enhancing and stabilizing microtubule formation, and analogs of Taxol™, such as Taxotere™. Compounds that have the basic taxane skeleton as a common structure feature, have also been shown to have the ability to arrest cells in the G2-M phases due to stabilized microtubules and may be useful for treating cancer in combination with the compounds described herein.

Further examples of anti-cancer agents include inhibitors of mitogen-activated protein kinase signaling, e.g., U0126, PD98059, PD184352, PD0325901, ARRY-142886, SB239063, SP600125, BAY 43-9006, wortmannin, or LY294002; Syk inhibitors; mTOR inhibitors; and antibodies (e.g., rituxan).

Other anti-cancer agents include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer agents include: 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Yet other anticancer agents that include alkylating agents, antimetabolites, natural products, or hormones, e.g., nitrogen mustards (e.g., mechloroethamine, cyclophosphamide, chlorambucil, etc.), alkyl sulfonates (e.g., busulfan), nitrosoureas (e.g., carmustine, lomustine, ete.), or triazenes (decarbazine, etc.). Examples of antimetabolites include but are not limited to folic acid analog (e.g., methotrexate), or pyrimidine analogs (e.g., Cytarabine), purine analogs (e.g., mercaptopurine, thioguanine, pentostatin).

Examples of natural products include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents include, but are not limited to, nitrogen mustards (e.g., mechloroethamine, cyclophosphamide, chlorambucil, meiphalan, etc.), ethylenimine and methylmelamines (e.g., hexamethylmelamine, thiotepa), alkyl sulfonates (e.g., busulfan), nitrosoureas (e.g., carmustine, lomustine, semustine, streptozocin, etc.), or triazenes (decarbazine, ete.). Examples of antimetabolites include, but are not limited to folic acid analog (e.g., methotrexate), or pyrimidine analogs (e.g., fluorouracil, floxuridine, Cytarabine), purine analogs (e.g., mercaptopurine, thioguanine, pentostatin.

Examples of hormones and antagonists include, but are not limited to, adrenocorticosteroids (e.g., prednisone), progestins (e.g., hydroxyprogesterone caproate, megestrol acetate, medroxyprogesterone acetate), estrogens (e.g., diethylstilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and compositions described herein for the treatment or prevention of cancer include platinum coordination complexes (e.g., cisplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

In some embodiments, provided herein is a method of treating lymphoma comprising administering a therapeutically effective amount of a compound described herein in combination with an antibody to CD20 and/or a CHOP (cyclophosphamide, doxorubicin, vincristine, and prednisone) therapy. In certain embodiments, provided herein is a method of treating leukemia comprising administering a therapeutically effective amount of a compound described herein in combination with ATRA, methotrexate, cyclophosphamide and the like.

### Pharmaceutical Compositions

In certain embodiments, pharmaceutical compositions are formulated in a conventional manner using one or more physiologically acceptable carriers including, e.g., excipients and auxiliaries which facilitate processing of the active compounds into preparations which are suitable for pharmaceutical use. In certain embodiments, proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

A pharmaceutical composition, as used herein, refers to a mixture of a ganglioside synthesis inhibitor described herein, with other chemical components, such as carriers, stabilizers, diluents, dispersing agents, suspending agents, thickening agents, and/or excipients. In certain instances, the pharmaceutical composition facilitates administration of the ganglioside synthesis inhibitor to an individual or cell. In certain embodiments of practicing the methods of treatment or use provided herein, therapeutically effective amounts of ganglioside synthesis inhibitors described herein are administered in a pharmaceutical composition to an individual having a disease, disorder, or condition to be treated. In specific embodiments, the individual is a human. As discussed herein, the ganglioside synthesis inhibitors described herein are either utilized singly or in combination with one or more additional therapeutic agents.

In certain embodiments, the pharmaceutical formulations described herein are administered to an individual in any manner, including one or more of multiple administration routes, such as, by way of non-limiting example, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound described herein are optionally manufactured in a conventional manner, such as, by way of example only, by means of conventional mixing, dissolving, granulating, dragee-making, levigating, emulsifying, encapsulating, entrapping or compression processes.

In certain embodiments, a pharmaceutical compositions described herein includes one or more ganglioside synthesis inhibitor described herein, as an active ingredient in free-acid or free-base form, or in a pharmaceutically acceptable salt form. In some embodiments, the compounds described herein are utilized as an N-oxide or in a crystalline or amorphous form (i.e., a polymorph). In certain embodiments, an active metabolite or prodrug of a compound described herein is utilized. In some situations, a compound described herein exists as tautomers. All tautomers are included within the scope of the compounds presented herein. In certain embodiments, a compound described herein exists in an unsolvated or solvated form, wherein solvated forms comprise any pharmaceutically acceptable solvent, e.g., water, ethanol, and the like. The solvated forms of the ganglioside synthesis inhibitors presented herein are also considered to be disclosed herein.

A “carrier” includes, in some embodiments, a pharmaceutically acceptable excipient and is selected on the basis of compatibility with ganglioside synthesis inhibitors disclosed herein and the release profile properties of the desired dosage form. Exemplary carrier materials include, e.g., binders, suspending agents, disintegration agents, filling agents, surfactants, solubilizers, stabilizers, lubricants, wetting agents, diluents, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Moreover, in certain embodiments, the pharmaceutical compositions described herein is formulated as a dosage form. As such, in some embodiments, provided herein is a dosage form comprising a ganglioside synthesis inhibitor described herein, suitable for administration to an individual. In certain embodiments, suitable dosage forms include, by way of non-limiting example, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

The pharmaceutical solid dosage forms described herein optionally include an additional therapeutic compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In some aspects, using coating procedures, such as those described in Remington's Pharmaceutical Sciences, 20th Edition (2000), a film coating is provided around the formulation of a ganglioside synthesis inhibitor described herein. In one embodiment, a ganglioside synthesis inhibitor described herein is in the form of a particle and some or all of the particles of the compound are coated. In certain embodiments, some or all of the particles of a ganglioside synthesis inhibitor described herein are microencapsulated. In some embodiment, the particles of the ganglioside synthesis inhibitor described herein are not microencapsulated and are uncoated.

In certain embodiments, the pharmaceutical composition described herein is in unit dosage forms suitable for single administration of precise dosages. In unit dosage form, the formulation is divided into unit doses containing appropriate quantities of one or more therapeutic compound. In some embodiments, the unit dosage is in the form of a package containing discrete quantities of the formulation. Non-limiting examples are packaged tablets or capsules, and powders in vials or ampoules. Aqueous suspension compositions are optionally packaged in single-dose non-reclosable containers. In some embodiments, multiple-dose re-closeable containers are used. In certain instances, multiple dose containers comprise a preservative in the composition. By way of example only, formulations for parenteral injection are presented in unit dosage form, which include, but are not limited to ampoules, or in multi-dose containers, with an added preservative.

These examples are provided for illustrative purposes only and not to limit the scope of the claims provided herein. The starting materials and reagents used for the processes, methods, and compositions described herein are synthesized or are obtained from commercial sources, such as, but not limited to, Sigma-Aldrich, Acros Organics, Fluka, and Fischer Scientific.

### EXAMPLES

### Example 1

### Cell-Based Assays for Identification of Ganglioside Biosynthesis Inhibitors

### Primary Assay

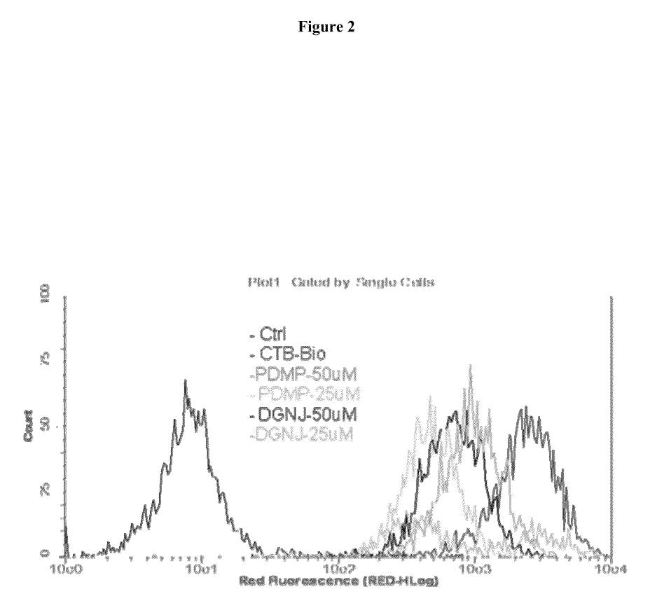

The impact of a ganglioside synthesis inhibitor on the ability of a protein (e.g., cholera toxin B-subunit (CTB)) to bind to gangliosides (e.g., GM₁ gangliosides) in mammalian cells was tested by incubating H82E cells in the absence and presence of the indicated concentrations of the glucoceramide synthase inhibitors 1-phenyl-2-hexadecanoylamino-3-pyrrolidino-1-propanol (PDMP) and N-butyldeoxynojirimycin (DGNJ). The control sample contained no CTB. The bound CTB was quantified using flow cytometry (FIG. 2). CTB was biotinylated and identified using PE-Cy5 Strepavidin (BD Pharmingen).

The ganglioside synthesis inhibitors are tested on at least three independent occasions, in duplicate over a dose range.

### Secondary Assay

Ganglioside specificity is then determined by probing with lectins and/or proteins that bind to other glycan classes (chondroitin sulfate, Heparan sulfate, O-linked, N-linked etc.).

### Example 2

### Quantitative Ganglioside Thin Layer Chromatography (TLC)

Mammalian cells (e.g., bovine brain tissue) were incubated in the presence of a ganglioside synthesis modulator. After 3 days of growth, the cells were harvested with trypsin, homogenized in a polytron homogenizer and the ganglioside containing fraction extracted 2 times with a mixture of chloroform, methanol and water and dried.

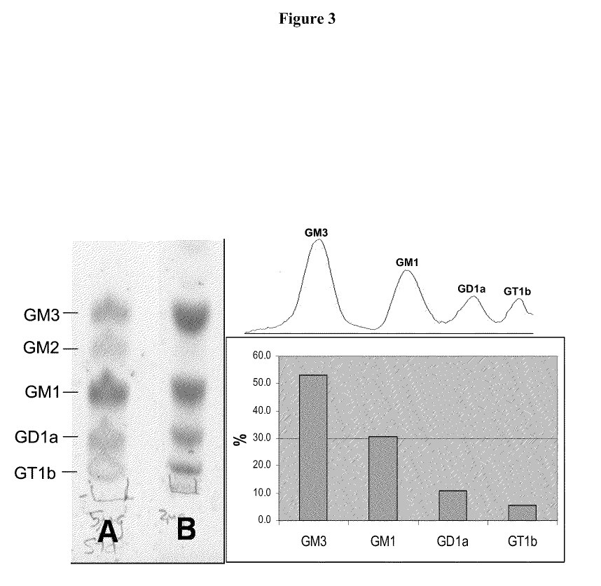

Sample or ganglioside standard aliquots were streaked on silica gel plates (e.g. Silica Gel 60 F254 glass backed plates, E. Merck) and developed in tanks pre-equilibrated with chloroform, methanol, water/0.2% CaCl2. Gangliosides are visualized using orcinol stain and ganglioside composition is quantified by densitometry (Molecular Imager GS-800, Bio-Rad, Hercules, Calif. and Quantity One software, Bio-Rad). Ganglioside production was normalized to sample weight prior to extraction. FIG. 3 shows a representative TLC, lane A is 5 μg of the Avanti ganglioside standard and lane B is bovine brain gangliosides extracted from 10 mg of tissue. The right panel shows quantification of lane B.

In some instances, endoglycoceramidase II from Rhodococcus is used after the glycans have been extracted from cells to hydrolyze gluceramide linkages and free glycans from the ceramides. The glycans are then analyzed as described herein.

The array of gangliosides that each cell type produces reflects the competition between enzymes for substrates to produce the mature lipid linked glycans. Therefore, inhibition of a ganglioside specific biosynthetic enzyme will produce an altered array of gangliosides. Analysis of the changes in ganglioside biosynthesis across a panel of cell lines lends insight into the drug mechanism of action. FIG. 4 shows the type and quantity of gangliosides produced by cells and reflects the competition for substrates by various biosynthetic enzymes; high levels of one ganglioside are generated at the expense of another.

In some instances, a reduction in the amounts of all A-series gangliosides and/or a reduction in the amounts of all B-series gangliosides with a concomitant increase or no change in the amounts of non-ganglioside glycolipids identifies a compound that is a GM3 synthase inhibitor. In some instances, a reduction of a subset of A-series gangliosides (e.g., GM1a, GD1a, and/or GT1a gangliosides) and/or a reduction of a subset of B-series gangliosides (e.g., GD1b, GT1b, and/or GQ1b gangliosides) with a concomitant increase or no change in the amounts of non-ganglioside glycolipids identifies a compound that is a GalTII inhibitor. In some instances, a reduction of all A-series gangliosides except GM3 gangliosides and/or a reduction of all B-series gangliosides except GD3 with a concomitant increase or no change in the amounts of non-ganglioside glycolipids identifies a compound that is a GM2/GD2 synthase inhibitor. In some instances, a reduction in some or all A-series gangliosides and/or a reduction in some or all B-series gangliosides with a decrease in the amounts of non-ganglioside glycolipids identifies a compound that is an inhibitor of an early glycolipid biosynthetic enzyme (i.e. glucosylceramide synthase) and is not a ganglioside specific inhibitor.

While preferred embodiments of the present invention have been shown and described herein, such embodiments are provided by way of example only. Various alternatives to the embodiments described herein are optionally employed in practicing the inventions. It is intended that the following claims define the scope of the invention and that methods and structures within the scope of these claims and their equivalents be covered thereby.

### Example 3

### ST3Gal 1 Assay

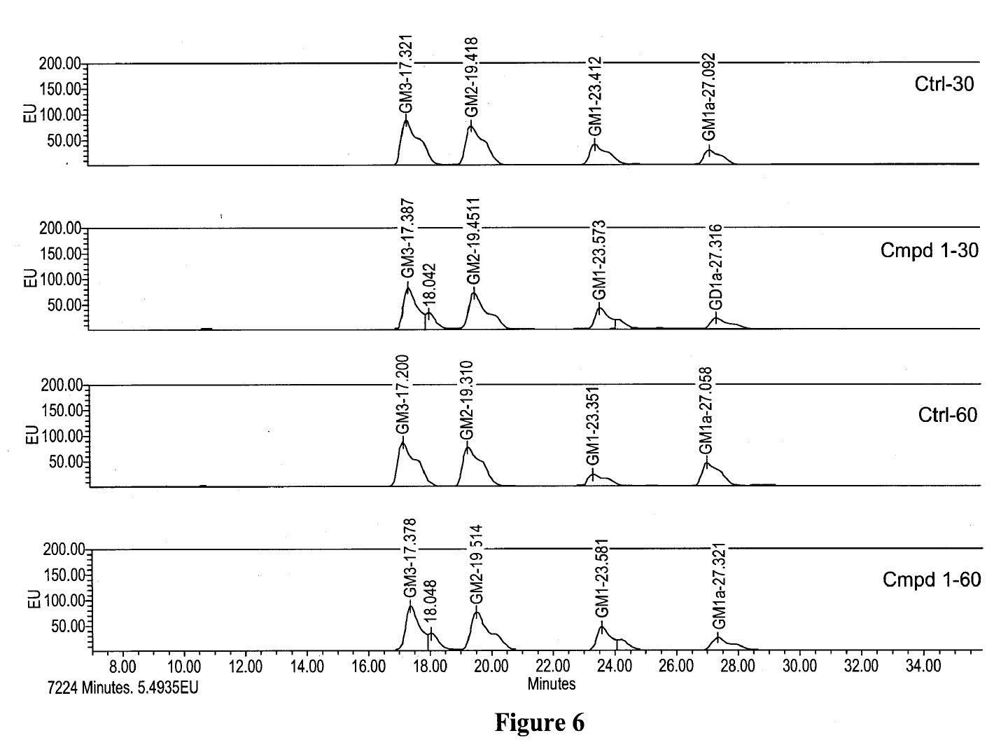

The enzyme ST3Gal 1 (rat recombinant a 2,3-Sialytransferase) assay was carried out with 2AA-labeled GSL standards as substrate. The complete incubation mixture contained the following components in a final volume of 20 ul: 50 mM cacodylate buffer, pH6.2, 10 mM MgCl2, 0.2% TX100, 2 mM CMP-sialic acid (CMP-SA), 100 ng 2AA-GM1/GD1b, 10 μU of ST3Gal1 enzyme. The reaction was carried out @37° C. for 30 min. Control reactions were done without CMP-SA. The reaction was analyzed by HPLC as described above. The % of product conversion was assessed by comparison to control samples. The enzyme titration curve was determined from 10, 3, 1, 0.3 and 0.1 μU of enzyme. The time course was determined using 10 μU of the enzyme, @ 0, 5, 10, 15, 30, 45, 60, 90, and 120 min. As expected, no inhibition was observed with early stage ganglioside inhibitors. FIG. 5 illustrates the activity of late stage selective ganglioside inhibitors. FIG. 6 illustrates an HPLC trace utilized to determine activity of late stage selective ganglioside inhibitors (FIG. 6 corresponds to compound 1, the results of which are demonstrated in FIG. 5).

### Example 4

### Cellular Activity

NCI-H82 cells were cultured in 6 well plates and treated with compounds at concentrations of 25, 12 and 6 uM in triplicates. PDMP (1-Phenyl-2-decanoylamino-3-morpholino-1-propanol, HCl, EMD #513100) at 25, 12, 6 uM was used as a control. After 96 hours of compound treatment the cells were harvested for ganglioside profiling. The suspension cells were centrifuged and the pellets washed once with PBS. Cells were resuspended in 1 ml of PBS; 1/100 of cells were taken to check the cell viability (Viacount) and for glycosphingolipid (GSL) quantitation and normalization. 1/10 of cells were taken for Flow cytometry (CTB-FACS) to check GM1 expression. For CTB-FACS, the resuspended cells were probed with biotinylated CTB (CTB-bio) diluted 1:2000 for 1 hour on ice. After washing to remove unbound CTB-bio, CTB-bio was detected with streptavidin-Cy5-PE diluted 1:1000. After washing to remove the unbound streptavidin-Cy5-PE the bound probe was quantified using flow cytometry. Compound doses are in uM. The Y-axis shows the % of control (untreated cells). Control cells were treated with vehicle only. The test compounds were tested on at least 3 independent occasions in duplicate over the indicated dose range.

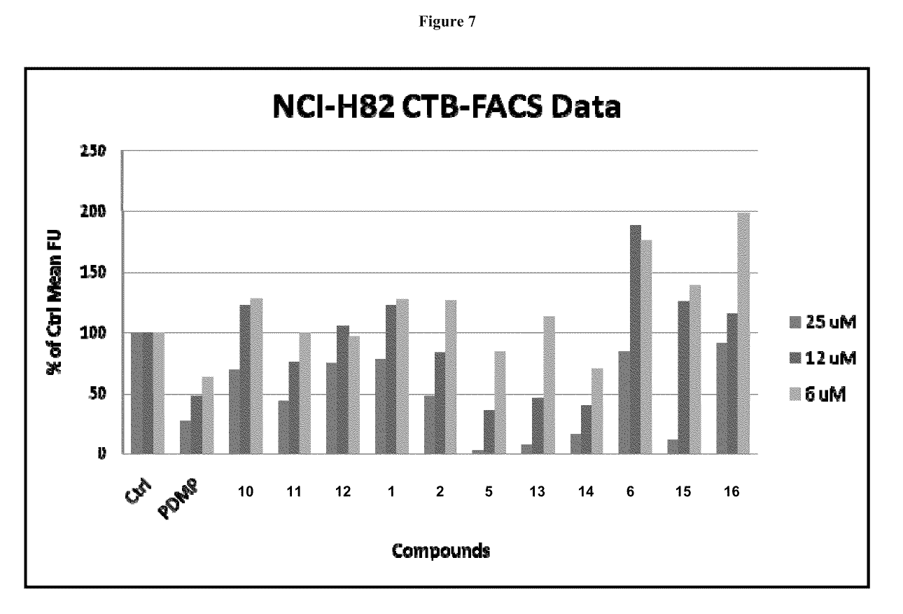

FIG. 7 demonstrates small molecule modulators (e.g., inhibitors) of the synthesis of gangliosides that are active within a cellular context.

### Example 5

### Example 5A

### Compound Treatment

NCI-H82 cells were cultured in 6 well plates and treated with compounds at a concentration of 25, 12 and 6 uM in triplicates. PDMP (1-Phenyl-2-decanoylamino-3-morpholino-1-propanol, HCl, EMD #513100) at 25, 12, 6 uM and DGNJ (N-(n-Butyl) deoxygalactonojirimycin, EMD #203994) at 50 uM were used as controls. After 96 hours of compounds treatment the cells were harvested for ganglioside profiling. The suspension cells were centrifuged and the pellets washed once with PBS. Cells were resuspended in 1 ml of PBS, 1/100 of cells were taken to check the cell viability (Viacount)) and for glycosphingolipid (GSL) quantitation and normalization. 1/10 of cells were taken for CTB-FACS to check GM1 expression. The remaining cells were subjected to the ganglioside extraction procedure follow the protocol described by R. Schnaar (Methods in Enzymology, 230: 348-370, 1994).

### Example 5B

### GSL Extraction

Briefly, the cells were pelleted by centrifugation and resuspended in cold distilled-water. The cell suspension was homogenized with Polytron homogenizer. Glycolipids were extracted with chloroform:methanol:water at 4:8:3 ratio. Glycolipids were partitioned by adding 0.173 volume of water to extracted the supernatant. The upper organic phase (glycolipids) was dried in a vacuum centrifuge (SpeedVac).

### Example 5C

### GSL Glycans Releasing and Fluorescence Labeling

Glycans were released from the glycolipids using the enzyme EGCase II (Sigma, Cat #E9030) as described by the manufacture. Released glycans were labeled with 2AA (2-Anthranilic acid, Sigma, Cat #A89804) following the procedures described by D. Neville (Analytical Biochemistry, 331 (2004) 275-282). Non-incorporated 2AA was removed by column chromatography (Discovery DPA-6S). Briefly, the extracted GSLs were digested with EGCase in a 10 ul reaction volume overnight. 40 ul of labeling mix (30 mg/ml 2-AA, 45 mg/ml NaCNBH4 in 4% NaAc and 2% boric acid in methano) were added. The reaction was carried at 80° C. for 45 min. 2AA-labeled glycans were purified by column chromatography (Discovery DPA-6S). The column was preequilibrated with 2×1 ml 97% acetonitrile (ACN) and the sample was loaded by adding 1 ml 97% ACN to the reaction mix. The column was washed 4× with 1 ml 97% ACN and the 2AA-labeled N-glycans were eluted with 2×0.6 ml water. After drying down (SpeedVac), the sample were resuspended and analyzed by normal phase high performance liquid chromatography (NP-HPLC).

### Example 5D

### GSL Analysis by HPLC

Purified 2AA-GSL glycans were separated on NP-HPLC using a 4.6×250 mm TSK Gel-Amide-80 column (Tosoh Bioscience). The chromatography system consisted of a Waters Alliance 2690/5 separation module and an in-line Waters 2475 fluorescence detector set at Exλ360 nm and Emλ425 nm. All chromatography was performed at 30° C. Solvent A was 20% 100 mM ammonium acetate pH3.85, 80% Acetonitrile, solvent B was 20% 100 mM ammonium acetate pH3.85, 20% ACN and 60% Milli-Q water. The gradient was run from 86% A to 54.7% A in 55 min at flow rate 0.8˜1.2 ml/min (Analytical Biochemistry, 331 (2004) 275-282). Chromatography data was processed using Waters Empower software. Glucose units were determined based on a 2AA-labeled glucose oligomer ladder (Ludger). All 2AA-GSL standards were prepared in house and labeled as glucose units.

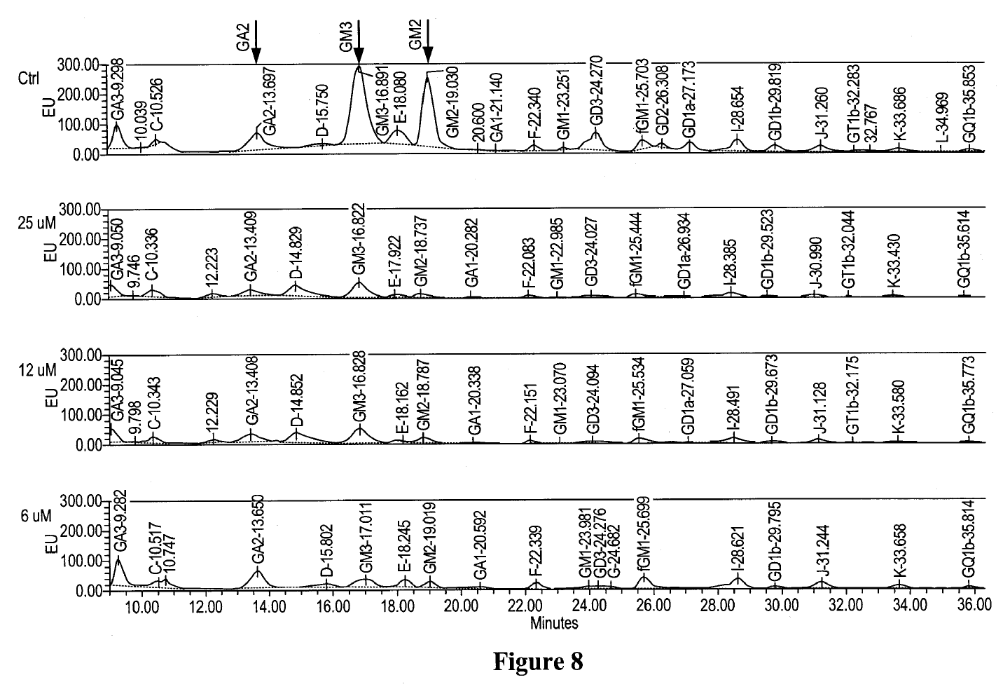

FIGS. 8-17 illustrate the ganglioside modulator activity observed for various compounds as determined according to the methods of Example 5.

FIGS. 8 and 9 illustrate the activity of PDMP. Inhibitors of galactoceramide synthase would have the advantage of not affecting glucoceramide levels.

A more specific inhibitor directed at blocking the biosynthesis of only the ganglioside subset of GSLs should reduce unwanted side effects due to the inhibition of all GSLs. Inhibitors of GM3 synthase (ST3Gal-V), GM2/GD2 synthase (b1-4 GalNAc transferase), GD3 synthase (ST8Sial-I), Gal TII, ST3GaI-II or downstream enzymes would affect only the ganglioside family.

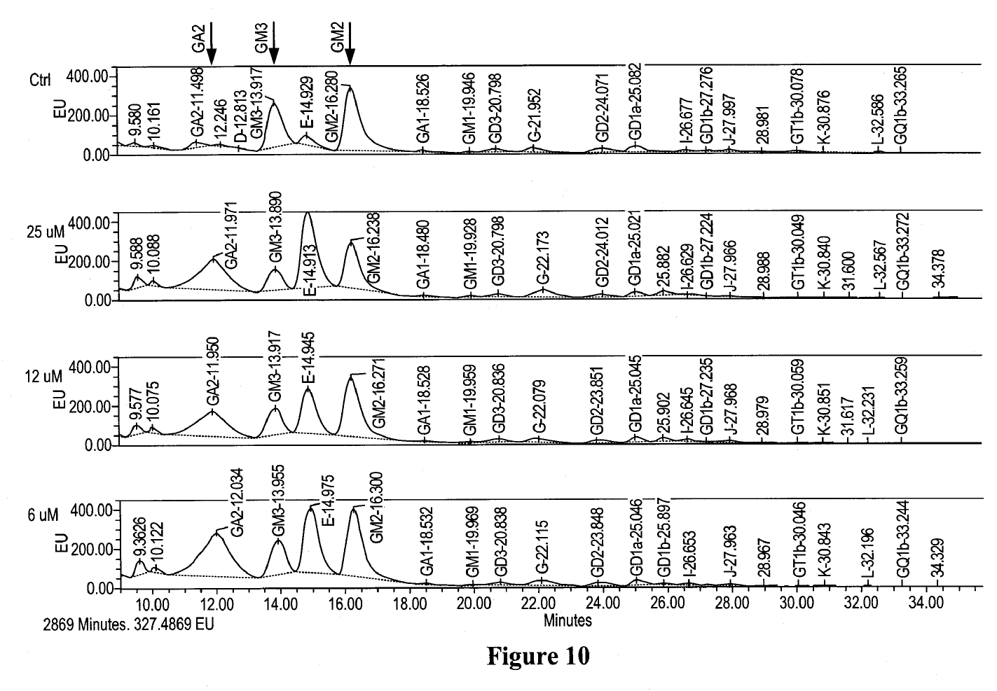

As opposed to PDMP, other selective inhibitors (e.g., late stage inhibitors) caused differential effects among the peaks indicating that specific enzymes downstream from the glucoceramide synthase were targeted. The exact distribution of ganglioside species (peaks) depends on the enzyme targeted by the inhibitor and by the specific cell type expression and intracellular distribution of the enzymes required for biosynthesis. Since the effects were downstream of glucoceramide formation only enzymes in the ganglioside pathway would be affected demonstrating that the inhibitors were specific modifiers of ganglioside expression. FIGS. 10-15 illustrate these effects.

Such selective inhibition is identified using any suitable process, such as described herein. For example, in some embodiments, specific modifiers preferentially inhibit synthesis of GM₃ and GD₃ relative to the other ganglioside species. FIGS. 16 and 17 illustrate a process described herein whereby preferential inhibition of GM₃ and GD₃ relative to other ganglioside species is identified. In some instances, ganglioside biosynthesis inhibitors that specifically target GM₃ and GD₃ provide a reduction in other gangliosides. For example, based on the biosynthetic pathway (see FIG. 4), the data in FIGS. 16 and 17 suggest that compounds that specifically target GM₃ and GD₃ provide for the reduction in other gangliosides as a result of the reduction in GM₃ and GD₃.

### Example 6

FIGS. 18-25 illustrate the dose dependent effects on individual gangliosides (individual HPLC peaks) of various compounds as determined according to the methods of Example 5, but with additional and slightly altered concentration levels used for test compound dosing. The results are displayed as the HPLC peaks areas of individual gangliosides, expressed as a % of the untreated peak areas.

### Example 7

Human gangliosidosis fibroblast cells are obtained e.g., from Coriell Institute for Medical Research (http://ccr.coriell.org). The primary cells are cultured in minimum essential medium (MEM) with 15% fetal bovine serum (FBS) as instructed by the supplier.

### Example 7A

### Compound Treatment

The fibroblast cells are cultured in 6 well plates at a density of 5.0E05/well in MEM, 15% FBS. The cells are treated with compounds at 30 uM on the next day of culture. The non-selective glycolipid inhibitors PDMP and DGNJ, which inhibit glucosylceramide synthase, are used as control. All the treatments are performed in triplicate. The medium is changed every 5 days with fresh compounds added. After 13 days of treatment with compounds, the cells are harvested for GSL analysis. The conditioned medium is removed, the monolayer is washed with PBS and detached with 5 mM EDTA in PBS. An aliquot of the cell suspension is taken for cell viability count using Viacount.

### Example 7B

### Glycosphingolipid (GSL) Extraction

The cells are spun down and the cell pellets are resuspended in water for GSL extraction. The cells are homogenized using a homogenizer at 6500 rpm for 30 seconds twice. Gangliosides are extracted from homogenates by using chloroform:methanol:water (4:8:3) followed by partitioning. The ganglioside containing upper phase is taken and dried down in a SpeedVac.

### Example 7C

### GSL Glycan Release and Fluorescence Labeling

Purified total GSLs are subjected to endo-glucoceramidase (EGCase II, Sigma) treatment to release free glycans. ˜10 ug protein equivalent GSL is digested with 1 mU EGCaseII in 15 ul incubation buffer containing 50 mM sodium acetate, pH 5.5 and 0.4% TX100. Digestion is performed at 37° C. overnight. Released free glycans are tagged with anthranilic Acid (2-AA, Sigma). A labeling mix is prepared freshly in 4% sodium acetate (NaAc.3H₂O) and 2% boric acid/methanol(w/v) by adding 45 mg NaBH₃CN first and then adding 30 mg 2AA in a 1 ml solution. 40 ul of this labeling mix is added directly into digestion mix. The labeling is carried out at 80° C. for 45˜60 min. Free labeling reagents are removed by passing the reaction mix through a Discovery DPA-6S (Sigma) column. The DPA-6S column (50 mg) is pre-equilibrated twice with 1 ml acetonitrile (ACN). The reaction mixture is cooled down, 1 ml of 97% ACN is added, and the sample is loaded onto the column. The column is washed with four times with 1 ml of 99% ACN and once with 0.5 ml of 97% ACN. 2AA-glycans are eluted in two time 600 ul water. The eluates are dried down in a SpeedVac and subjected to HPLC analysis.

### Example 7D

### GSL Analysis by HPLC

Purified 2-AA-labeled oligosaccharides are analyzed by HPLC an method as described by Neville et al. (Anal. Biochem, 2004, 331:275-282). 2-AA-oligosaccharides are separated by NP-HPLC using a 5 μm 4.6×250 mm TSK gel-Amide 80 column (Tosoh) on a Waters Alliance 2690 separation module equipped with a Waters 2475 fluorescence detector set at Exλ360 nm and Emλ425 nm. Solvent A is 80% ACN and 20% 100 mM ammonium acetate (AmAc), pH 3.85. Solvent B is 60% Milli-Q water, 20% ACN and 20% 100 mM AmAc, pH 3.85. The gradient profile is listed below. Glucose unites are determined based on a 2-AA-labeled glucose ladder (Ludger, UK).

| Time | Flow rate | Buffer A | Buffer B |
|---|---|---|---|
| [min] | [ml/min] | [%] | [%] |
| 0 | 0.8 | 86 | 14 |
| 6 | 0.8 | 86 | 14 |
| 35 | 0.8 | 54.7 | 47.3 |
| 37 | 0.8 | 5 | 95 |
| 39 | 1 | 5 | 95 |
| 41 | 1 | 86 | 14 |
| 42 | 1.2 | 86 | 14 |
| 54 | 1.2 | 86 | 14 |
| 55 | 0.8 | 86 | 14 |

FIGS. 26-35 illustrate the reduction of GM₂ storage in primary human fibroblasts from patients with Sandhoff or Tay-Sachs disease by various compounds as determined according to the methods of Example 7.

FIGS. 26 and 27 illustrate the activity of the known non-selective glycolipid inhibitors PDMP and DGNJ, respectively. A more specific inhibitor directed at blocking the biosynthesis of only the ganglioside subset of GSLs should reduce unwanted side effects due to the inhibition of all GSLs. Inhibitors of for example GM3 synthase (ST3Gal-V), GM2/GD2 synthase (b1-4 GalNAc transferase), GD3 synthase (ST8Sial-I), Gal TII, ST3Gal-II or other downstream enzymes would affect only the ganglioside family. Selective inhibitors (e.g., late stage inhibitors) also reduce GM₂ storage in primary human fibroblasts from patients with Sandhoff and Tay-Sachs disease. FIGS. 28-35 illustrate this effect.

### Example 8

### Method of Treatment

Human Clinical Trial of the Safety and/or Efficacy of selective ganglioside biosynthesis inhibitor (e.g., a compound of FIGS. 36A-36I, or a pharmaceutically acceptable salt thereof) therapy.

Objective: To determine the safety, pharmacokinetics, and efficacy of administered selective ganglioside biosynthesis inhibitor (e.g., a compound of FIGS. 36A-36I, or a pharmaceutically acceptable salt thereof).

Study Design: This will be a Phase I, single-center, open-label, randomized dose escalation study followed by a Phase II study in cancer patients with a cancer that can be biopsied (e.g., neuroblastoma, or lung cancer). Patients should not have had exposure to a ganglioside biosynthesis inhibitor prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC>30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive (e.g., intravenous, oral, ip, or the like) selective ganglioside biosynthesis inhibitor (e.g., a compound of FIGS. 36A-36I, or a pharmaceutically acceptable salt thereof) daily for 5 consecutive days or 7 days a week. Doses of selective ganglioside biosynthesis inhibitor (e.g., a compound of FIGS. 36A-36I, or a pharmaceutically acceptable salt thereof) may be held or modified for toxicity based on assessments as outlined below. Treatment repeats every 28 days in the absence of unacceptable toxicity. Cohorts of 3-6 patients receive escalating doses of selective ganglioside biosynthesis inhibitor (e.g., a compound of FIGS. 36A-36I, or a pharmaceutically acceptable salt thereof) until the maximum tolerated dose (MTD) for the selective ganglioside biosynthesis inhibitor (e.g., a compound of FIGS. 36A-36I, or a pharmaceutically acceptable salt thereof) is determined. The MTD is defined as the dose preceding that at which 2 of 3 or 2 of 6 patients experience dose-limiting toxicity. Dose limiting toxicities are determined in any suitable manner, e.g., according to the definitions and standards set by the National Cancer Institute (NCI) Common Terminology for Adverse Events (CTCAE) Version 3.0 (Aug. 9, 2006).

Phase II: Patients receive selective ganglioside biosynthesis inhibitor (e.g., a compound of FIGS. 36A-36I, or a pharmaceutically acceptable salt thereof) as in phase I at the MTD determined in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling: Serial blood is drawn by direct vein puncture before and after administration of selective ganglioside biosynthesis inhibitor (e.g., a compound of FIGS. 36A-36I, or a pharmaceutically acceptable salt thereof). Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). Patients also undergo cancer/tumor biopsy to assess changes in progenitor cancer cell phenotype and clonogenic growth by flow cytometry, Western blotting, and IHC, and for changes in cytogenetics by FISH or TaqMan PCR for specific chromosomal translocations. After completion of study treatment, patients are followed periodically for 4 weeks.

### Example 9

### Method of Treatment

Human Clinical Trial of the Safety and/or Efficacy of selective ganglioside biosynthesis inhibitor (e.g., a compound of FIGS. 36A-36I, or a pharmaceutically acceptable salt thereof) therapy.

Objective: To determine the safety, pharmacokinetics, and efficacy of administered selective ganglioside biosynthesis inhibitor (e.g., a compound of FIGS. 36A-36I, or a pharmaceutically acceptable salt thereof).

Study Design: This will be a Phase I, single-center, open-label, non-randomized dose escalation study followed by a Phase II study in gangliosidosis patients (for example Tay-Sachs and Sandhoff disease patients). The diagnosis of gangliosidosis is confirmed by demonstration of profound deficiency of β-hexosaminidase A or A&B in peripheral blood leukocytes or cultured skin fibroblasts. Patients should not have had exposure to a selective ganglioside biosynthesis inhibitor, glucoceramide synthase inhibitor, or enzyme replacement therapy prior to the study entry. Patients must not have received other investigational agents within 3 months of study initiation. Fertile patients must agree to use adequate contraception throughout the study and for 3 months after cessation of treatment with selective ganglioside biosynthesis inhibitor (e.g., a compound of FIGS. 36A-36I, or a pharmaceutically acceptable salt thereof). Patients must not have a history of significant gastrointestinal disorders, including clinically significant diarrhea without definable cause within 3 months of baseline visit. Patients must not be anemic (hemoglobin<11 g/dl, and/or hematocrit<34%). All subjects are evaluated for safety and all blood collections for pharmacokinetic analysis are collected as scheduled. All studies are performed with institutional ethics committee approval and patient consent.

Phase I: Patients receive (e.g., intravenous, oral, ip, or the like) selective ganglioside biosynthesis inhibitor (e.g., a compound of FIGS. 36A-36I, or a pharmaceutically acceptable salt thereof) daily for 4 weeks. Cohorts of 3-6 patients receive escalating doses of selective ganglioside biosynthesis inhibitor (e.g., a compound of FIGS. 36A-36I, or a pharmaceutically acceptable salt thereof). Escalation will not be performed until all patients in the previous dose cohort have been treated for 4 weeks and until results obtained 4 weeks after treatment initiation do not reveal toxicity. Doses of selective ganglioside biosynthesis inhibitor (e.g., a compound of FIGS. 36A-36I, or a pharmaceutically acceptable salt thereof) may be held or modified for toxicity based on assessments as outlined below. Dose escalation is considered complete, if 2 patients experience a Grade 3 Adverse Event (AE) or if 1 patient experiences Grade 4 AE at a particular cohort.

Phase II: Patients receive selective ganglioside biosynthesis inhibitor (e.g., a compound of FIGS. 36A-36I, or a pharmaceutically acceptable salt thereof) as in phase I at a suitable dose below the dose used in the final cohort. Treatment continues throughout a 24-month study period during which clinical (which includes safety and tolerability) assessments are performed.

Blood Sampling: Serial blood is drawn by direct vein puncture before and after administration of selective ganglioside biosynthesis inhibitor (e.g., a compound of FIGS. 36A-36I, or a pharmaceutically acceptable salt thereof). Venous blood samples (5 mL) for determination of serum concentrations are obtained in-hospital during a 24-hour period. Each serum sample is divided into two aliquots. All serum samples are stored at −20° C. Serum samples are shipped on dry ice.

Pharmacokinetics: Patients undergo plasma/serum sample collection for pharmacokinetic evaluation in-hospital during a 24-hour period. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response: The primary outcome measure is safety and tolerability, based on conventional laboratory and clinical assessments. The secondary outcome measure is the assessment of changes in β-hexosaminidase A and B activities in plasma and peripheral blood leukocytes. In addition, changes in volume loss and signal intensity from baseline MRI, change in single-voxel N-acetylaspartate (NAA) from baseline MRS, change in neuropsychological testing from baseline, change in nerve conduction, and change in neurological examination from baseline are assessed.

What is claimed is:

1. A process for modifying the cellular population of a ganglioside, the process comprising contacting a cell having at least one ganglioside with an effective amount of a selective late-stage ganglioside biosynthesis inhibitor, the selective ganglioside biosynthesis inhibitor being active in a mammalian cell.

2. The process of claim 1, wherein the selective late-stage ganglioside biosynthesis inhibitor is a non-carbohydrate inhibitor.

3. The process of claim 1, wherein the selective ganglioside biosynthesis inhibitor has a molecular weight of less than 700 g/mol.

4. The process of claim 1, wherein the process:
a. reduces the ratio of gangliosides containing mono (α 2,3) sialylation of the (β 1,4) galactose residue in the ceramide linked core compared to gangliosides containing no sialylation of the (β 1,4) galactose residue in the ceramide linked core; and/or
b. reduces the ratio of gangliosides containing mono (α 2,3) sialylation of the (β 1,4) galactose residue in the ceramide linked core compared to gangliosides containing a di-sialylation of the (β 1,4) galactose residue in the ceramide linked core.

5. The process of claim 1, wherein the process:
a. reduces the ratio of gangliosides containing di-sialylation of the (β 1,4) galactose residue in the ceramide linked core compared to gangliosides containing no sialylation of the (β 1,4) galactose residue in the ceramide linked core, and/or
b. reduces the ratio of gangliosides containing di-sialylation of the (β 1,4) galactose residue in the ceramide linked core compared to gangliosides containing mono (α 2,3) sialylation of the (β 1,4) galactose residue in the ceramide linked core.

6. The process of claim 1, wherein the process reduces the cellular population of GD_{1b}, GD₂ gangliosides, GD₃ gangliosides, or a combination.

7. The process of claim 1, wherein the process reduces the cellular population of GM₁ gangliosides, GM₂ gangliosides, GM₃ gangliosides or a combination.

8. The process of claim 1, wherein the selective ganglioside biosynthesis inhibitor inhibits ST3Gal-V transferase, β1-4 GalNAc transferase, β1-3Gal-II transferase ST3Gal-I/II transferase, ST8Sial-I transferase, or a combination thereof.

9. The process of claim 8, wherein the selective ganglioside biosynthesis inhibitor directly inhibits the ST3Gal-V transferase, β1-4 GalNAc transferase, β1-3Gal-II transferase ST3Gal-I/II transferase, ST8Sial-I transferase, or a combination thereof.

10. The process of claim 8, wherein the selective ganglioside biosynthesis inhibitor indirectly inhibits the ST3Gal-V transferase, β1-4 GalNAc transferase, β1-3Gal-II transferase ST3Gal-I/II transferase, ST8Sial-I transferase, or a combination thereof.

11. The process of claim 1, wherein the process reduces the ratio of gangliosides containing a terminal (β1,4) linked GalNAc linked to the (β1,4) galactose residue compared to gangliosides with a (β1,4) galactose lacking a GalNAc.

12. The process of claim 1, wherein the process reduces the ratio of gangliosides containing an unmodified (β1,3) linked galactose compared to gangliosides containing a terminal (β1,4) GalNAc.

13. The process of claim 1, wherein the cell is a cancer cell or a cell having abnormal ganglioside accumulation.

14. The process of claim 1, wherein the cell is present in an individual diagnosed with or suspected of having cancer, inflammation or an inflammatory disease, pathogen entry, or lysosomal storage disease.

15. The process of claim 14, wherein the cell is present in an individual diagnosed with or suspected of having melanoma, neuroblastoma, breast cancer or lung cancer.

16. The process of claim 14, wherein the cell is present in an individual diagnosed with or suspected of having a lysosomal storage disease, the lysosomal storage disease being Tay-Sachs, Sandhoff, AB variant, GM1 gangliosidosis, or Neimann-Pick.

17. A composition comprising a population of human serum gangliosides, the population comprising less than 34 mol. % α 2,8-linked sialic acid containing gangliosides.

18. A composition comprising a population of human serum gangliosides, the population comprising greater than 3 mol. % O series gangliosides.

19. A composition comprising a population of human serum gangliosides, the population comprising less than 15 mol. % (β1,3) linked galactose containing gangliosides.

20. A composition comprising a population of human serum gangliosides, the population comprising less than 23 mol. % of (β1,4) linked GalNac gangliosides.
